(19) United States
(12) Patent Application Publication
(10) Pub. No.: US 2025/0283076 A1
(43) Pub. Date: Sep. 11, 2025

(54) TREATMENT OF CARDIOVASCULAR DISEASE

(71) Applicant: ARGONAUTE RNA LIMITED, Bristol (GB)

(72) Inventors: Stella Khan, Bristol (GB); Daniel Mitchell, Bristol (GB); Michael Khan, Bristol (GB)

(73) Assignee: ARGONAUTE RNA LIMITED, Bristol (GB)

(21) Appl. No.: 18/691,775

(22) PCT Filed: Sep. 13, 2022
(86) PCT No.: PCT/EP2022/075355

§ 371 Date: Mar. 13, 2024

(30) Foreign Application Priority Data
Sep. 14, 2021 (GB) 2113104.0
May 18, 2022 (GB) 2207239.1

(51) Int. Cl. C12N 15/113

(52) U.S. Cl. CPC C12N 15/113; C12N 15/1137; C12N 2310/11; C12N 2310/14; C12N 2310/312; C12N 2310/315; C12N 2310/335; C12N 2310/3519; C12N 2310/531

(57) ABSTRACT

This disclosure relates to a nucleic acid comprising a double stranded RNA molecule comprising sense and antisense strands and further comprising a single stranded DNA molecule covalently linked to at least the 5′ end of either the sense or antisense RNA part of the molecule wherein the double stranded inhibitory RNA targets genes associated with cardiovascular disease in the treatment hypercholesterolemia and diseases associated with hypercholesterolemia such as cardiovascular disease.

### CROSS REFERENCE TO RELATED APPLICATIONS

This is the U.S. National Stage of International Application No. PCT/EP2022/075355, filed Sep. 13, 2022, which was published in English under PCT Article 21(2), which claims the benefit of GB Application No. 2207239.1 filed May 18, 2022 and GB Application No. 2113104.0, filed Sep. 14, 2021.

### FIELD OF THE DISCLOSURE

This disclosure relates to a nucleic acid comprising a double stranded RNA molecule comprising sense and antisense strands and further comprising a single stranded DNA molecule covalently linked to at least 5′ end of either the sense or antisense RNA part of the molecule wherein the double stranded inhibitory RNA targets of cardiovascular disease genes; pharmaceutical compositions comprising said nucleic acid molecule and methods for the treatment of diseases associated with increased levels of expression of cardiovascular disease genes, for example hypercholesterolemia.

### INCORPORATION OF ELECTRONIC SEQUENCE LISTING

The electronic sequence listing, submitted herewith as an XML file named 4860P-US.xml (772,353 bytes), created on Sep. 26, 2024, is herein incorporated by reference in its entirety.

### BACKGROUND TO THE DISCLOSURE

Cardiovascular disease associated with hypercholesterolemia, for example ischaemic cardiovascular disease, is a common condition and results in heart disease and a high incidence of death and morbidity and can be a consequence of poor diet, obesity, or an inherited dysfunctional gene. For example, high levels of lipoprotein (a) and other lipoproteins, is associated with atherosclerosis. Cholesterol is essential for membrane biogenesis in animal cells. The lack of water solubility means that cholesterol is transported around the body in association with lipoproteins. Apolipoproteins form together with phospholipids, cholesterol and lipids which facilitate the transport of lipids such as cholesterol, through the bloodstream to the different parts of the body. Lipoproteins are classified according to size and can form HDL (High-density lipoprotein), LDL (Low-density lipoprotein), IDL (intermediate-density lipoprotein), VLDL (very low-density lipoprotein) and ULDL (ultra-low-density lipoprotein) lipoproteins.

Lipoproteins change composition throughout their circulation comprising different ratios of apolipoproteins A (ApoA), B (ApoB), C (ApoC), D(ApoD) or E (ApoE), triglycerides, cholesterol and phospholipids. For example, ApoB is the main apolipoprotein of ULDL and LDL and has two isoforms apoB-48 and apoB-100. Both ApoB isoforms are encoded by one single gene and wherein the shorter ApoB-48 gene is produced after RNA editing of the ApoB-100 transcript at residue 2180 resulting in the creation of a stop codon. ApoB-100 is the main structural protein of LDL and serves as a ligand for a cell receptor which allows transport of, for example, cholesterol into a cell.

Familial hypercholesterolemia is an orphan disease and results from elevated levels of LDL cholesterol (LDL-C) in the blood. The disease is an autosomal dominant disorder with both the heterozygous (350-550 mg/dL LDL-C) and homozygous (650-1000 mg/dL LDL-C) states resulting in elevated LDL-C. The heterozygous form of familial hypercholesterolemia is around 1:500 of the population. The homozygous state is much rarer and is approximately 1:1,000,000. The normal levels of LDL-C are in the region 130 mg/dL.

Hypercholesterolemia is particularly acute in paediatric patients which if not diagnosed early can result in accelerated coronary heart disease and premature death. If diagnosed and treated early the child can have a normal life expectancy. In adults, high LDL-C, either because of mutation or other factors, is directly associated with increased risk of atherosclerosis which can lead to coronary artery disease, stroke, or kidney disease. Lowering levels of LDL-C is known to reduce the risk of atherosclerosis and associated conditions. LDL-C levels can be lowered initially by administration of statins which block the de novo synthesis of cholesterol by inhibiting the HMG-CoA reductase. Some subjects can benefit from combination therapy which combines a statin with other therapeutic agents such as ezetimibe, colestipol or nicotinic acid. However, expression and synthesis of HMG-CoA reductase adapts in response to the statin inhibition and increases over time, thus the beneficial effects are only temporary or limited after statin resistance is established.

There is therefore a desire to identify alternative therapies that can be used alone or in combination with existing therapeutic approaches to control cardiovascular disease because of elevated LDL-C.

A technique to specifically ablate gene function is through the introduction of double stranded inhibitory RNA, also referred to as small inhibitory or interfering RNA (siRNA), into a cell which results in the destruction of mRNA complementary to the sequence included in the siRNA molecule. The siRNA molecule comprises two complementary strands of RNA (a sense strand and an antisense strand) annealed to each other to form a double stranded RNA molecule. The siRNA molecule is typically, but not exclusively, derived from exons of the gene which is to be ablated. Many organisms respond to the presence of double stranded RNA by activating a cascade that leads to the formation of siRNA. The presence of double stranded RNA activates a protein complex comprising RNase Ill which processes the double stranded RNA into smaller fragments (siRNAs, approximately 21-29 nucleotides in length) which become part of a ribonucleoprotein complex. The siRNA acts as a guide for the RNase complex to cleave mRNA complementary to the antisense strand of the siRNA thereby resulting in destruction of the mRNA.

The inhibition of expression of lipoprotein (a) is known and the use of inhibitory RNA to silence expression of lipoprotein (a) is also known. For example, WO2019/092283 discloses the identification of specific siRNA sequences that target knock down of mRNA encoding lipoprotein (a) and their use in the treatment of cardiovascular diseases linked to elevated lipoprotein (a) expression such as coronary heart disease, aortic stenosis or stroke. Similarly, U.S. Pat. No. 9,932,586 discloses specific siRNA sequences that target lipoprotein (a) expression and their use in the treatment of cardiovascular diseases linked to elevated lipoprotein (a) expression such as Buerger's disease, coronary heart disease, renal artery stenosis, hyperapobetalipoproteinemia, cerebrovascular atherosclerosis, cerebrovascular disease, and venous thrombosis.

Over expression of APOC III is associated with atherosclerosis and type 2 diabetes. For example, WO2003/020765 discloses a vaccination approach to the control of atherosclerosis using immunogens derived from ApoCIII polypeptide and its use in controlling atherosclerotic plaques in coronary and cerebrovascular disease. A similar vaccination approach is disclosed 5 in WO2004/080375 and WO2001/064008. In WO2014/205449 and WO2014/179626 is disclosed the use of antisense oligonucleotides to improve insulin sensitivity and treat type II diabetes by targeting APOCIII expression.

Furthermore, WO2007/136989 and WO2005/019418 each disclose the use of antisense compounds directed to DGAT to regulate expression of DGAT2 and treat conditions that would benefit from reduction in DGAT2 expression in relation to conditions that would benefit from reduction in serum triglyceride levels such as hypercholesterolemia, cardiovascular disease, type II diabetes and metabolic syndrome. WO2018/093966 discloses the use of RNA silencing 10 directed to DGAT2 and diglyceride acyltransferase 1(DGAT1) to treat obesity and obesity associated diseases such as hypercholesterolemia, cardiovascular disease, type II diabetes and metabolic syndrome. Similarly, WO2005/044981 discloses the use of siRNA to target DGAT2 amongst many other gene targets and their use in the treatment of diseases that would benefit from triglyceride regulation.

This disclosure relates to a nucleic acid molecule comprising a double stranded inhibitory RNA that is modified by the inclusion of a short DNA part linked to at least the 5′ end of either the sense or antisense inhibitory RNA and which forms a hairpin structure. The double stranded inhibitory RNA uses solely or predominantly natural nucleotides and does not require modified nucleotides or sugars that prior art double stranded RNA molecules typically utilise to improve pharmacodynamics and pharmacokinetics. The disclosed double stranded inhibitory RNAs have activity in silencing cardiovascular gene targets with potentially fewer side effects.

### STATEMENTS OF THE INVENTION

According to an aspect of the invention there is provided a nucleic acid molecule comprising
a first part that comprises a double stranded inhibitory ribonucleic acid (RNA) molecule comprising a sense strand and an antisense strand; and
a second part that comprises a single stranded deoxyribonucleic acid (DNA) molecule, wherein the 3′ end of said single stranded DNA molecule is covalently linked to the 5′ end of the sense strand of the double stranded inhibitory RNA molecule or wherein the 3′ end of the single stranded DNA molecule is covalently linked to the 5′ of the antisense strand of the double stranded inhibitory RNA molecule, characterized in that the double stranded inhibitory RNA comprises a sense nucleotide sequence that encodes a part of a cardiovascular gene target associated with cardiovascular disease and wherein said single stranded DNA molecule comprises a nucleotide sequence that is adapted over at least part of its length to anneal by complementary base pairing to a part of said single stranded DNA to form a double stranded DNA structure wherein said double stranded inhibitory RNA consists of natural nucleotides.

According to an aspect of the invention there is provided a nucleic acid molecule comprising
a first part that comprises a double stranded inhibitory ribonucleic acid (RNA) molecule comprising a sense strand and an antisense strand; and
a second part that comprises a single stranded deoxyribonucleic acid (DNA) molecule, wherein the 3′ end of said single stranded DNA molecule is covalently linked to the 5′ end of the sense strand of the double stranded inhibitory RNA molecule or wherein the 3′ end of the single stranded DNA molecule is covalently linked to the 5′ of the antisense strand of the double stranded inhibitory RNA molecule, characterized in that the double stranded inhibitory RNA comprises a sense nucleotide sequence that encodes a part of a cardiovascular gene target associated with cardiovascular disease, or a polymorphic sequence variant thereof, and wherein said single stranded DNA molecule comprises a nucleotide sequence that is adapted over at least part of its length to anneal by complementary base pairing to a part of said single stranded DNA to form a double stranded DNA structure comprising a stem and a loop domain, characterized in that said nucleic acid molecule comprises N-acetylgalactosamine and said double stranded inhibitory RNA consists of natural nucleotides.

A “polymorphic sequence variant” is a gene sequence that varies by one, two, three or more nucleotides.

In a preferred embodiment of the invention wherein the 3′ end of said single stranded DNA molecule is covalently linked to the 5′ end of the sense strand of the double stranded inhibitory RNA molecule.

In a preferred embodiment of the invention wherein the 3′ end of said single stranded DNA molecule is covalently linked to the 5′ end of the antisense strand of the double stranded inhibitory RNA molecule.

In a preferred embodiment of the invention single stranded DNA molecule is covalently linked to the 5′ end of said sense strand and the 5′ end of said antisense strand.

In an alternative embodiment of the invention said single stranded DNA molecule is covalently linked to the 5′ end of said sense strand, the 3′ end of said sense strand.

In a preferred embodiment of the invention said loop portion comprises a region comprising the nucleotide sequence GNA or GNNA, wherein each N independently represents guanine (G), thymidine (T), adenine (A), or cytosine (C).

In a preferred embodiment of the invention said loop domain comprises G and C nucleotide bases.

In an alternative embodiment of the invention said loop domain comprises the nucleotide sequence GCGAAGC.

In a preferred embodiment of the invention said single stranded DNA molecule comprises the nucleotide sequence 5TCACCTCATCCCGCGAAGC 3′ (SEQ ID NO 387 and 251).

In a preferred embodiment of the invention said single stranded DNA molecule comprises the nucleotide sequence 5′ CGAAGCGCCCTACTCCACT 3′. (SEQ ID NO 130)

In a preferred embodiment of the invention said single stranded DNA molecule comprises the nucleotide sequence 5′ GCGAAGCCCCTACTCCACT 3′ (SEQ ID NO 400).

The inhibitory RNA molecules comprise or consist of natural nucleotide bases that do not require chemical modification. Moreover, in some embodiments of the invention, wherein the crook DNA molecule is linked to the 3′ end of the sense strand of said double stranded inhibitory RNA, the antisense strand is optionally provided with at least a two-nucleotide base overhang sequence. The two-nucleotide overhang sequence can correspond to nucleotides encoded by the target or are non-encoding. The two-nucleotide overhang can be two nucleotides of any sequence and in any order, for example UU, AA, UA. AU. GG, CC, GC, CG, UG, GU, UC, CU, and TT.

In a preferred embodiment of the invention said inhibitory RNA molecule comprises a two-nucleotide overhang comprising or consisting of deoxythymidine dinucleotide (dTdT).

In a preferred embodiment of the invention said dTdT overhang is positioned at the 5′ end of said antisense strand.

In an alternative preferred embodiment of the invention said dTdT overhang is positioned at the 3′ end of said antisense strand.

In a preferred embodiment of the invention said dTdT overhang is positioned at the 5′ end of said sense strand.

In an alternative preferred embodiment of the invention said dTdT overhang is positioned at the 3′ end of said sense strand.

In a preferred embodiment of the invention said sense and/or said antisense strands comprises internucleotide phosphorothioate linkages.

In a preferred embodiment of the invention said sense strand comprises internucleotide phosphorothioate linkages.

Preferably, the 5′ and/or 3′ terminal two nucleotides of said sense strand comprises two internucleotide phosphorothioate linkage.

In a preferred embodiment of the invention said antisense strand comprises internucleotide phosphorothioate linkages.

Preferably, the 5′ and/or 3′ terminal two nucleotides of said antisense strand comprises two internucleotide phosphorothioate linkages.

In an alternative preferred embodiment of the invention said single stranded DNA molecule comprises one or more internucleotide phosphorothioate linkages.

In a preferred embodiment of the invention said nucleic acid molecule comprises a vinylphosphonate modification.

In a preferred embodiment of the invention said vinylphosphonate modification is to the 5′ terminal phosphate of said sense RNA strand.

In a preferred embodiment of the invention said vinylphosphonate modification is to the 5′ terminal phosphate of said antisense RNA strand.

In a preferred embodiment of the invention said double stranded inhibitory RNA molecule is between 10 and 40 nucleotides in length.

In a preferred embodiment of the invention said double stranded inhibitory RNA molecule is between 17 and 29 nucleotides in length.

In a preferred embodiment of the invention said double stranded inhibitory RNA molecule is 19 to 21 nucleotides in length. Preferably, 19 nucleotides in length.

In a preferred embodiment of the invention said cardiovascular gene target is Human Lipoprotein (a).

In an alternative embodiment of the invention said double stranded inhibitory RNA molecule comprises an antisense nucleotide sequence selected from the group consisting of: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 or 34.

In a preferred embodiment of the invention said double stranded inhibitory RNA molecule comprises an antisense nucleotide sequence comprising SEQ ID NO: 41 and a sense nucleotide sequence comprising SEQ ID NO: 49, wherein said single stranded DNA molecule is covalently linked to the 5′ end of the sense strand of the double stranded inhibitory RNA molecule.

In a preferred embodiment of the invention said double stranded inhibitory RNA molecule comprises an antisense nucleotide sequence comprising SEQ ID NO: 4 and a sense nucleotide sequence comprising SEQ ID NO: 44, wherein said single stranded DNA molecule is covalently linked to the 5′ end of the antisense strand of the double stranded inhibitory RNA molecule.

In a preferred embodiment of the invention said double stranded inhibitory RNA molecule comprises an antisense nucleotide sequence comprising SEQ ID NO: 5 and a sense nucleotide sequence comprising SEQ ID NO: 46, wherein said single stranded DNA molecule is covalently linked to the 5′ end of the antisense strand of the double stranded inhibitory RNA molecule.

In an alternative preferred embodiment of the invention said cardiovascular gene target is Human Apolipoprotein C III (Apo C III).

Preferably, said nucleic acid molecule comprises an RNA strand comprising a nucleotide sequence selected from the group consisting of: SEQ ID NO: 60, 61, 62, 63, 64, 65, 66, 67, 68, 69, 70, 71, 72, 73, 74, 75, 76, 77, 78 and 79.

In a preferred embodiment of the invention said nucleic acid molecule comprises an RNA strand comprising a nucleotide sequence selected from the group consisting of: SEQ ID NO: 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249 and 250.

Preferably said nucleic acid molecule comprises an RNA strand comprising a nucleotide sequence selected from the group consisting of: SEQ ID NO: 50, 51, 52, 53, 54, 55, 56, 57, 58, 80, 81, 82, 83, 84, 85, 86, 87, 88 and 89.

In an alternative preferred embodiment of the invention said cardiovascular gene target is Human diglyceride acyltransferase 2 (DGAT2).

Preferably, said nucleic acid comprises an RNA strand comprising a nucleotide sequence selected from the group consisting of: 100, 101, 102, 103, 104, 105, 106, 107, 108, 109, 110, 111, 112, 113, 114, 115, 116, 117, 118 and 119.

Preferably, said nucleic acid comprises an RNA strand comprising a nucleotide sequence selected from the group consisting of: 131, 132, 133, 134, 135, 136, 137, 138, 139, 140, 141, 142, 143, 144, 145, 146, 147, 148, 149, 150, 151, 152, 153, 154, 156, 157, 158, 159, 160, 161, 162, 163, 164, 165, 166, 167, 168, 169 and 170.

Preferably said nucleic acid comprises an RNA strand comprising a nucleotide sequence selected from the group consisting of: SEQ ID NO: 90, 91, 92, 93, 94, 95, 96, 97, 98, 99, 120, 121, 122, 123, 124, 125, 126, 127, 128 and 129.

In a preferred embodiment of the invention said cardiovascular gene target is Human PCSK9.

In a preferred embodiment of the invention said nucleic acid molecule comprises an RNA strand comprising a nucleotide sequence selected from the group consisting of: SEQ ID NO: 171, 172, 173, 174, 175, 176, 177, 178, 179, 180, 181, 182, 183, 184, 185, 186, 187, 189 and 190.

In a preferred embodiment of the invention said nucleic acid molecule comprises an RNA strand comprising a nucleotide sequence selected from the group consisting of: SEQ ID NO: 191, 192, 193, 194, 195, 196, 197, 198, 199, 200, 201, 202, 203, 204, 205, 206, 207, 208, 209 and 210.

In a preferred embodiment of the invention said nucleic acid molecule comprises an RNA strand comprising a nucleotide sequence selected from the group consisting of: SEQ ID NO: 255, 256, 257, 258, 259, 260, 261, 262, 263 and 264.

In a preferred embodiment of the invention said nucleic acid molecule comprises an RNA strand comprising a nucleotide sequence selected from the group consisting of: SEQ ID NO: 265, 266, 267, 268, 269, 270, 271, 272, 273 and 274.

In a preferred embodiment of the invention said nucleic acid molecule comprises an RNA strand comprising a nucleotide sequence selected from the group consisting of: SEQ ID NO: 275, 276, 277, 278, 279, 280, 281, 282, 283, 284, 285, 286, 287, 288, 289, 290, 292, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329 and 330.

In a preferred embodiment of the invention said nucleic acid molecule comprises an RNA strand comprising a nucleotide sequence selected from the group consisting of: SEQ ID NO: 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 285 and 386.

In a preferred embodiment of the invention said cardiovascular gene target is Human Apolipoprotein B.

In a preferred embodiment of the invention said nucleic acid molecule comprises an RNA strand comprising a nucleotide sequence selected from the group consisting of: SEQ ID NO: 499, 500, 453, 502, 503, 457, 505, 506, 462, 508, 509, 467, 511, 512, 472, 514, 515, 477, 517 518, 482, 520, 521, 487, 523, 524 and 492.

In a preferred embodiment of the invention said nucleic acid molecule comprises a RNA strand comprising a nucleotide sequence selected from the group consisting of: 450, 501, 455, 504, 460, 507, 465, 510, 470, 513, 475, 516, 480, 519, 485, 522, 490 and 525.

In a preferred embodiment of the invention said nucleic acid molecule comprises a RNA strand comprising or consisting of a nucleotide sequence, or polymorphic sequence variant, set forth in table 1.

In a preferred embodiment of the invention said nucleic acid molecule comprises a RNA strand comprising or consisting of a nucleotide sequence, or polymorphic sequence variant, set forth in table 2.

In a preferred embodiment of the invention said nucleic acid molecule comprises a RNA strand comprising or consisting of a nucleotide sequence, or polymorphic sequence variant, set forth in table 3.

In a preferred embodiment of the invention said nucleic acid molecule comprises a RNA strand comprising or consisting of a nucleotide sequence, or polymorphic sequence variant, set forth in table 4.

In a preferred embodiment of the invention said nucleic acid molecule comprises a RNA strand comprising or consisting of a nucleotide sequence, or polymorphic sequence variant, set forth in table 5.

In a preferred embodiment of the invention said nucleic acid molecule comprises a RNA strand comprising or consisting of a nucleotide sequence, or polymorphic sequence variant, set forth in table 8.

In a preferred embodiment of the invention said nucleic acid molecule comprises a RNA strand comprising or consisting of a nucleotide sequence, or polymorphic sequence variant, set forth in table 10.

In a preferred embodiment of the invention said nucleic acid molecule comprises a RNA strand comprising or consisting of a nucleotide sequence, or polymorphic sequence variant, set forth in table 14.

In a preferred embodiment of the invention said nucleic acid molecule comprises a RNA strand comprising or consisting of a nucleotide sequence, or polymorphic sequence variant, set forth in table 15.

In a preferred embodiment of the invention said nucleic acid molecule comprises or consists of between 19 and 21 contiguous nucleotides of the nucleotide sequence set forth in SEQ ID NO:388.

In a preferred embodiment of the invention said nucleic acid molecule is covalently linked to N-acetylgalactosamine.

In a further embodiment of the invention N-acetylgalactosamine is linked to either the antisense part of said inhibitory RNA or the sense part of said inhibitory RNA.

Preferably, N-acetylgalactosamine is linked to the 5′ terminus is of said sense RNA. In an alternative embodiment of the invention N-acetylgalactosamine is linked to the 3′ terminus of said sense RNA.

In an alternative preferred embodiment of the invention said N-acetylgalactosamine is linked to the 3′ terminus of said antisense RNA.

In a preferred embodiment of the invention N-acetylgalactosamine is monovalent.

In a preferred embodiment of the invention N-acetylgalactosamine is divalent.

In an alternative embodiment of the invention N-acetylgalactosamine is trivalent.

In a preferred embodiment of the invention said nucleic acid molecule is covalently linked to a molecule comprising the structure:

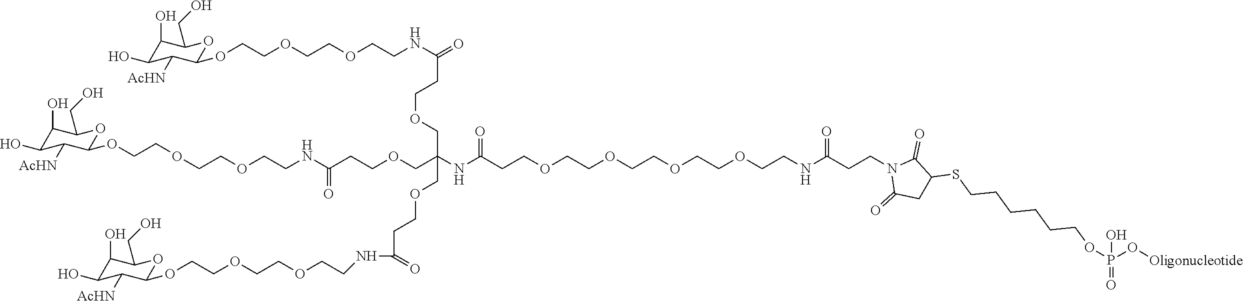

In an alternative embodiment of the invention said nucleic acid molecule is covalently linked to a molecule comprising the structure:

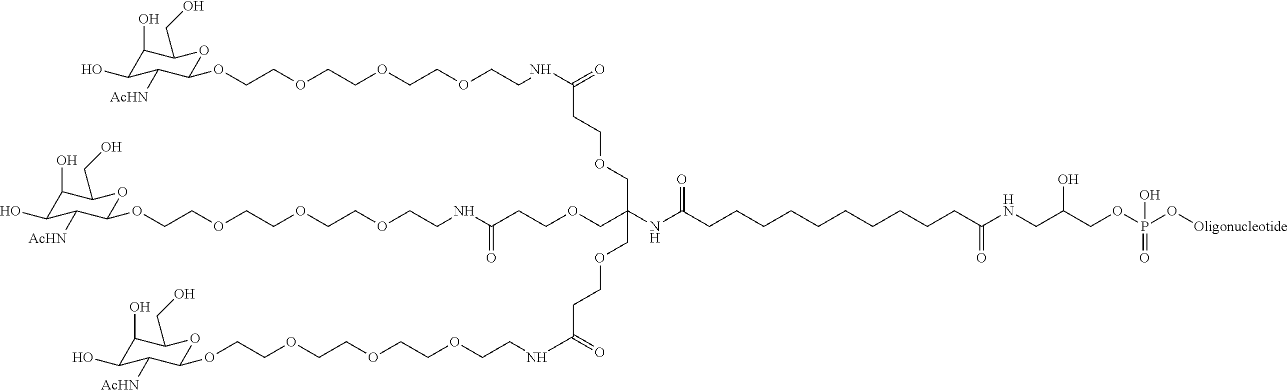

In an alternative embodiment of the invention said nucleic acid molecule is covalently linked to a molecule comprising the structure:

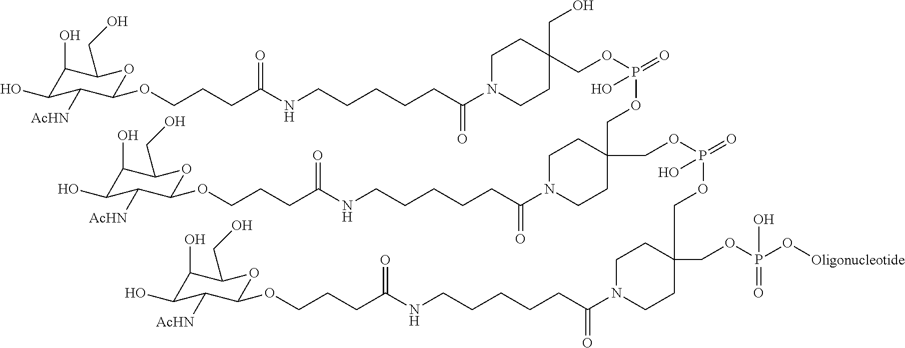

In an alternative embodiment of the invention said nucleic acid molecule is covalently linked to a molecule comprising the structure:

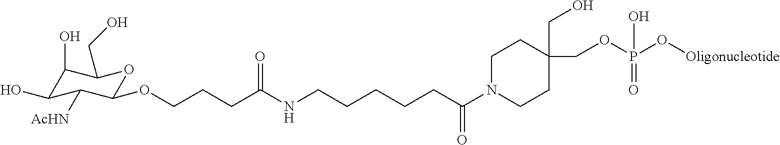

In an alternative preferred embodiment of the invention said nucleic acid molecule is covalently linked to a molecule comprising N-acetylgalactosamine 4-sulfate.

According to a further aspect of the invention there is provided a pharmaceutical composition comprising at least one nucleic acid molecule according to the invention.

In a preferred embodiment of the invention said composition further includes a pharmaceutical carrier and/or excipient.

When administered the compositions of the present invention are administered in pharmaceutically acceptable preparations. Such preparations may routinely contain pharmaceutically acceptable concentrations of salt, buffering agents, preservatives, compatible carriers and optionally other therapeutic agents, such as cholesterol lowering agents, which can be administered separately from the nucleic acid molecule according to the invention or in a combined preparation if a combination is compatible.

The combination of a nucleic acid according to the invention and the other, different therapeutic agent is administered as simultaneous, sequential or temporally separate dosages.

The therapeutics of the invention can be administered by any conventional route, including injection or by gradual infusion over time. The administration may, for example, be oral, intravenous, intraperitoneal, intramuscular, intracavity, subcutaneous, transdermal or transepithelial.

The compositions of the invention are administered in effective amounts. An “effective amount” is that amount of a composition that alone, or together with further doses, produces the desired response. In the case of treating a disease, such as cardiovascular disease, the desired response is inhibiting or reversing the progression of the disease. This may involve only slowing the progression of the disease temporarily, although more preferably, it involves halting the progression of the disease permanently. This can be monitored by routine methods.

Such amounts will depend, of course, on the particular condition being treated, the severity of the condition, the individual patient parameters including age, physical condition, size and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

The pharmaceutical compositions used in the foregoing methods preferably are sterile and contain an effective amount of a nucleic acid molecule according to the invention for producing the desired response in a unit of weight or volume suitable for administration to a patient. The response can, for example, be measured by determining regression of cardiovascular disease and decrease of disease symptoms etc.

The doses of the nucleic acid molecule according to the invention administered to a subject can be chosen in accordance with different parameters, in particular in accordance with the mode of administration used and the state of the subject. Other factors include the desired period of treatment. If a response in a subject is insufficient at the initial doses applied, higher doses (or effectively higher doses by a different, more localized delivery route) may be employed to the extent that patient tolerance permits. It will be apparent that the method of detection of the nucleic acid according to the invention facilitates the determination of an appropriate dosage for a subject in need of treatment.

In general, doses of the nucleic acid molecules herein disclosed of between 1 nM-1 μM generally will be formulated and administered according to standard procedures. Preferably doses can range from 1 nM-500 nM, 5 nM-200 nM, 10 nM-100 nM. Other protocols for the administration of compositions will be known to one of ordinary skill in the art, in which the dose amount, schedule of injections, sites of injections, mode of administration and the like vary from the foregoing. The administration of compositions to mammals other than humans, (e.g. for testing purposes or veterinary therapeutic purposes), is carried out under substantially the same conditions as described above. A subject, as used herein, is a mammal, preferably a human, and including a non-human primate or a transgenic mammal adapted for expression of human lipoprotein(a).

When administered, the pharmaceutical preparations of the invention are applied in pharmaceutically acceptable amounts and in pharmaceutically acceptable compositions. The term “pharmaceutically acceptable” means a non-toxic material that does not interfere with the effectiveness of the biological activity of the active ingredients. Such preparations may routinely contain salts, buffering agents, preservatives, compatible carriers, and optionally other therapeutic agents e.g., statins. When used in medicine, the salts should be pharmaceutically acceptable, but non-pharmaceutically acceptable salts may conveniently be used to prepare pharmaceutically acceptable salts thereof and are not excluded from the scope of the invention. Such pharmacologically and pharmaceutically acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically acceptable salts can be prepared as alkaline metal or alkaline earth salts, such as sodium, potassium, or calcium salts.

Compositions may be combined, if desired, with a pharmaceutically acceptable carrier. The term “pharmaceutically acceptable carrier” as used herein means one or more compatible solid or liquid fillers, diluents or encapsulating substances which are suitable for administration into a human. The term “pharmaceutically acceptable carrier” in this context denotes an organic or inorganic ingredient, natural or synthetic, with which the active ingredient is combined to facilitate, for example, solubility and/or stability. The components of the pharmaceutical compositions also are capable of being co-mingled with the molecules of the present invention, and with each other, in a manner such that there is no interaction which would substantially impair the desired pharmaceutical efficacy.

The pharmaceutical compositions may contain suitable buffering agents, including acetic acid in a salt; citric acid in a salt; boric acid in a salt; and phosphoric acid in a salt. The pharmaceutical compositions also may contain, optionally, suitable preservatives.

The pharmaceutical compositions may conveniently be presented in unit dosage form and may be prepared by any of the methods well-known in the art of pharmacy. All methods include the step of bringing the active agent into association with a carrier which constitutes one or more accessory ingredients. In general, the compositions are prepared by uniformly and intimately bringing the active compound into association with a liquid carrier, a finely divided solid carrier, or both, and then, if necessary, shaping the product. Compositions suitable for oral administration may be presented as discrete units, such as capsules, tablets, lozenges, each containing a predetermined amount of the active compound.

Compositions suitable for parenteral administration conveniently comprise a sterile aqueous or non-aqueous preparation of nucleic acid, which is preferably isotonic with the blood of the recipient. This preparation may be formulated according to known methods using suitable dispersing or wetting agents and suspending agents. The sterile injectable preparation also may be a sterile injectable solution or suspension in a non-toxic parenterally acceptable diluent or solvent, for example, as a solution in 1, 3-butane diol. Among the acceptable solvents that may be employed are water, Ringer's solution, and isotonic sodium chloride solution. In addition, sterile, fixed oils are conventionally employed as a solvent or suspending medium. For this purpose, any bland fixed oil may be employed including synthetic mono- or di-glycerides. In addition, fatty acids such as oleic acid may be used in the preparation of injectables. Carrier formulation suitable for oral, subcutaneous, intravenous, intramuscular, etc. administrations can be found in Remington's Pharmaceutical Sciences, Mack Publishing Co., Easton, PA.

In a further preferred embodiment of the invention said pharmaceutical composition comprises at least one further, different, therapeutic agent.

In a preferred embodiment of the invention said further therapeutic agent is a statin.

Statins are commonly used to control cholesterol levels in subjects that have elevated LDL-C. Statins are effective in preventing and treating those subjects that are susceptible and those that have cardiovascular disease. The typical dosage of a statin is in the region 5 to 80 mg but this is dependent on the statin and the desired level of reduction of LDL-C required for the subject suffering from high LDL-C. However, expression and synthesis of HMG-CoA reductase, the target for statins, adapts in response to statin administration thus the beneficial effects of statin therapy are only temporary or limited after statin resistance is established.

Preferably said statin is selected from the group consisting of atorvastatin, fluvastatin, lovastatin, pitvastatin, pravastatin, rosuvastatin and simvastatin.

In a preferred embodiment of the invention said further therapeutic agent is ezetimibe.

Optionally, ezetimibe is combined with at least one statin, for example simvastatin.

In an alternative preferred embodiment of the invention said further therapeutic agent is selected from the group consisting of fibrates, nicotinic acid, cholestyramine.

In a further alternative preferred embodiment of the invention said further therapeutic agent is a therapeutic antibody, for example, evolocumab, bococizumab or alirocumab.

According to a further aspect of the invention there is provided a nucleic acid molecule or a pharmaceutical composition according to the invention for use in the treatment or prevention of a subject that has or is predisposed to hypercholesterolemia or diseases associated with hypercholesterolemia.

In a preferred embodiment of the invention said subject is a paediatric subject.

A paediatric subject includes neonates (0-28 days old), infants (1-24 months old), young children (2-6 years old) and prepubescent [7-14 years old] children.

In an alternative preferred embodiment of the invention said subject is an adult subject.

In a preferred embodiment of the invention the hypercholesterolemia is familial hypercholesterolemia.

In a preferred embodiment of the invention familial hypercholesterolemia is associated with elevated levels of lipoprotein (a) expression.

In a preferred embodiment of the invention said subject is resistant to statin therapy.

In a preferred embodiment of the invention said disease associated with hypercholesterolemia is selected from the group consisting of: stroke prevention, hyperlipidaemia, cardiovascular disease, atherosclerosis, coronary heart disease, aortic stenosis, cerebrovascular disease, peripheral arterial disease, hypertension, metabolic syndrome, type II diabetes, non-alcoholic fatty acid liver disease, non-alcoholic steatohepatitis, Buerger's disease, renal artery stenosis, hyperapobetalipoproteinemia, cerebrovascular atherosclerosis, cerebrovascular disease and venous thrombosis.

According to a further aspect of the invention there is provided a method to treat a subject that has or is predisposed to hypercholesterolemia comprising administering an effective dose of a nucleic acid or a pharmaceutical composition according to the invention thereby treating or preventing hypercholesterolemia.

In a preferred method of the invention said subject is a paediatric subject.

In an alternative preferred method of the invention said subject is an adult subject.

In a preferred method of the invention the hypercholesterolemia is familial hypercholesterolemia.

In a preferred method of the invention familial hypercholesterolemia is associated with elevated levels of lipoprotein (a) expression.

In a preferred method of the invention said subject is resistant to statin therapy.

In a preferred method of the invention said disease associated with hypercholesterolemia is selected from the group consisting of: stroke prevention, hyperiipidaemia, cardiovascular disease, atherosclerosis, coronary heart disease, aortic stenosis, cerebrovascular disease, peripheral arterial disease, hypertension, metabolic syndrome, type 11 diabetes, non-alcoholic fatty acid liver disease, non-alcoholic steatohepatitis, Buerger's disease, renal artery stenosis, hyperapobetalipoproteinemia, cerebrovascular atherosclerosis, cerebrovascular disease and venous thrombosis.

Throughout the description and claims of this specification, the words “comprise” and “contain” and variations of the words, for example “comprising” and “comprises”, means “including but not limited to” and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with an aspect, embodiment or example of the invention are to be understood to be applicable to any other aspect, embodiment or example described herein unless incompatible therewith.

An embodiment of the invention will now be described by example only and with reference to the following figures.

### BRIEF SUMMARY OF THE DRAWINGS

FIG. 1 Serum stability assays showing target PCSK9 mRNA levels in HepG2 cells following transfection of siRNA compounds. HepG2 cells were transfected with the following siRNAs after 30 mins or 2 hr incubation at 37 C in water, 10% FBS or 10% human serum: modified Inclisiran [white bar; SEQ ID NOs: 494 and 495], unmodified ‘Inclisiran’ with no Crook [grey bar; SEQ ID NOs: 389 and 390], unmodified Inclisiran with 3′SS Crook [hatched bar; SEQ ID NOs: 496 and 390], unmodified Inclisiran with 5′ SS ‘reversed hairpin’ Crook [spotted bar; SEQ ID NOs: 497 and 390], or unmodified Inclisiran with 5′SS Crook [hatched bar; SEQ ID NOs: 498 and 390]. PCSK9 mRNA levels were quantified by RT-qPCR analysis. Controls include ‘no siRNA’ treatment [black bar];

FIG. 2 Serum stability assays showing target PCSK9 mRNA levels in HepG2 cells following transfection of siRNA compounds. HepG2 cells were transfected with the following siRNAs after a 2 hr incubation at 37 C in water, 10%, 20% or 50% FBS: modified Inclisiran [white bar; SEQ ID NOs: 494 and 495], unmodified ‘Inclisiran’ with no Crook [grey bar; SEQ ID NOs: 389 and 390], unmodified Inclisiran with 5′ SS ‘reversed hairpin’ Crook [spotted bar; SEQ ID NOs: 497 and 390], or unmodified Inclisiran with 5′SS Crook [striped bar; SEQ ID NOs: 498 and 390]. PCSK9 mRNA levels were quantified by RT-qPCR analysis. Controls include ‘no siRNA’ pre-treatment [black bar];

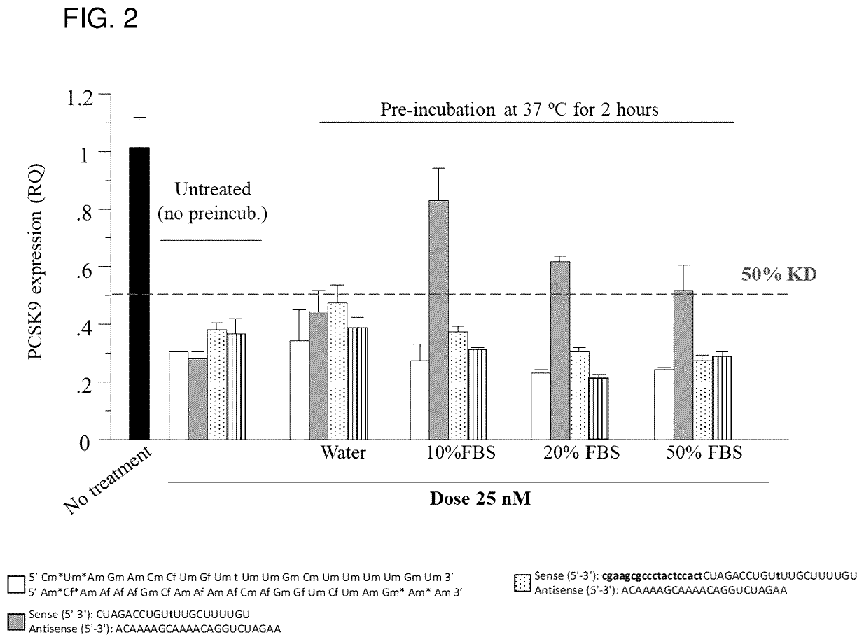

FIG. 3 Serum stability assays showing target PCSK9 mRNA levels in HepG2 cells following transfection of siRNA compounds. HepG2 cells were transfected with the following siRNAs after a 4-hr incubation at 37 C in water, 10% FBS or 10% human serum: modified Inclisiran [white bar; SEQ ID NOs: 494 and 495], unmodified ‘Inclisiran’ with no Crook [grey bar; SEQ ID NOs: 389 and 390], unmodified Inclisiran with 5′ SS ‘reversed hairpin’ Crook [spotted bar; SEQ ID NOs: 497 and 390], or unmodified Inclisiran with 5′SS Crook [striped bar; SEQ ID NOs: 498 and 390]. PCSK9 mRNA levels were quantified by RT-qPCR analysis. Controls include ‘no siRNA’ pre-treatment [black bar];

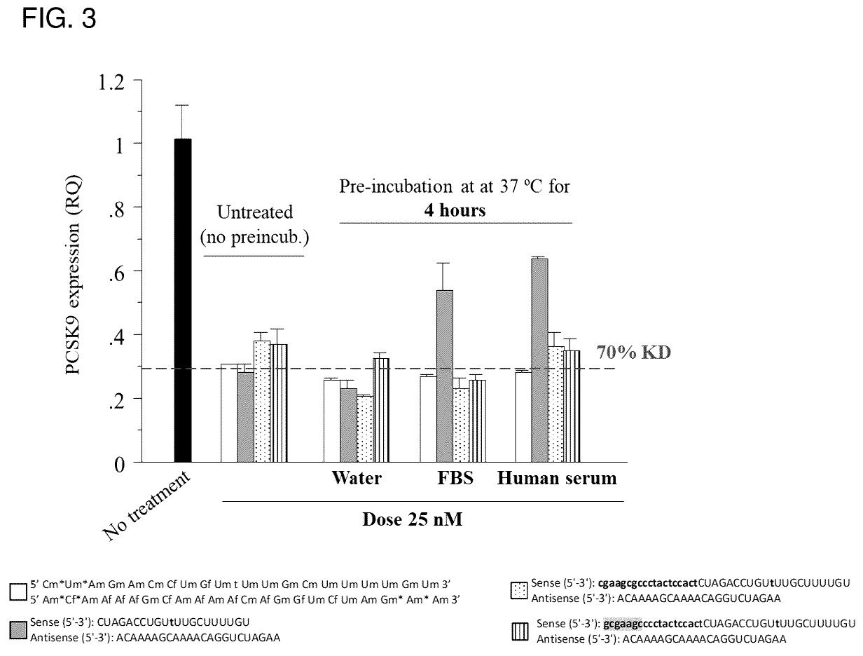

FIG. 4 Serum stability assays showing target PCSK9 mRNA levels in HepG2 cells following transfection of siRNA (termed PC8-PC18) compounds. HepG2 cells were transfected with the following unmodified PC8-18 siRNAs after a 2-hr incubation at 37 C in water, 10% FBS or 10% human serum: siRNA35 with no Crook [white bar; SEQ ID NOs: 176 and 272], siRNA36 with no Crook but including dTdT overhangs on 3′ SS & 3′ AS [grey bar; SEQ ID NOs: 421 and 422], siRNA37 with Crook on 3′ SS [spotted bar; SEQ ID NOs: 423 and 272], siRNA38 with Crook on 3′ AS [vertical striped bar; SEQ ID NOs: 278 and 424], siRNA39 with Crook on 3′ SS and dTdT overhang on 3′ AS [hatched bar SEQ ID NOs: 423 and 422], siRNA41 with 5′ SS ‘reversed hairpin’ Crook [horizontal stripe bar: SEQ ID NOs: 425 and 272], or siRNA42 with Crook on 5′ SS and dTdT overhang on 3′ AS [spots of black background bar; SEQ ID NOs: 425 and 422]. PCSK9 mRNA levels were quantified by RT-qPCR analysis. Controls include ‘no siRNA’ pre-treatment [black bar];

FIG. 5A In vivo silencing of liver PCSK9 mRNA following administration of unmodified siRNA compounds (termed PC2-PC12) Groups of 5 mice for each treatment group were injected subcutaneously (SC) with either vehicle [black bar], compound A (no Crook; white bar; SEQ ID NOs: 172 and 252), compound G (Crook on 5′ end of sense strand (SS); spotted bar; SEQ ID NOs: 428 and 252), or compound H (Crook on 3′ end of SS; grey bar; SEQ ID NOs: 253 and 252). Each compound was given at either 2 mg/kg or 10 mg/kg, and following sacrifice, levels of liver PCSK9 mRNA by RT-qPCR were measured at two time points (day 2 and day 7) and

FIG. 5B Serum stability assays showing target PCSK9 mRNA levels in HepG2 cells following transfection of siRNA compounds A, G and H, used in mouse in vivo study (FIG. 5A). HepG2 cells were transfected with siRNA compounds A, G, or H after 30 min or 2 hr incubation at 37 C in water, 10% FBS or 10% human serum: compound A (no Crook; white bar’ SEQ ID NOs: 172 and 252), compound G (Crook on 5′ end of sense strand (SS); spotted bar; SEQ ID NOs: 428 and 252), or compound H (Crook on 3′ end of SS; grey bar; SEQ ID NOs: 253 and 252). PCSK9 mRNA levels were quantified by RT-qPCR analysis. Controls include ‘no siRNA’ [black bar], and ‘no serum’ pre-treatment.

FIG. 5C Serum stability assays showing target PCSK9 mRNA levels in HepG2 cells following transfection of siRNA compounds A, G and H, used in mouse in vivo study (FIG. 5A). HepG2 cells were transfected with siRNA compounds A. G, or H after a 2 hr incubation at 37 C in water, 20% or 50% human serum: compound A (no Crook; white bar; SEQ ID NOs: 172 and 252), compound G (Crook on 5′ end of sense strand (SS); spotted bar; SEQ ID NOs: 428 and 252), or compound H (Crook on 3′ end of SS; grey bar; SEQ ID NOs: 253 and 252). PCSK9 mRNA levels were quantified by RT-qPCR analysis. Controls include ‘no siRNA’ [black bar], and ‘no serum’ pre-treatment.

### MATERIALS AND METHODS

### Hepg2 Reverse Transfection

Duplex siRNAs synthesized by Bio-Synthesis (Lewisville, TX) (Table 1), were resuspended in Nuclease-free water (Invitrogen™ AM9932) to generate a stock solution of 10 μM. For serum stability assay, stock siRNAs were incubated at 37° C. in vehicle (nuclease-free water), 10% fetal bovine serum (FBS) or in various concentrations (10%-80%) of human serum (HS) for 2 hours. After pre-incubation in serum or vehicle, siRNAs were transfected into HepG2 cells in a 384-well plate (Thermo Scientific™ 164688) at a concentration of 25 nM using 0.15 μL of Lipofectamine RNAiMAX (Invitrogen™ 13778075) per well. Transfected cells were incubated at 37° C. and 5% CO₂. Cells receiving no siRNA treatment were used as control.

### Free-Uptake and Transfection in Primary Mouse Hepatocytes

Mouse hepatocytes (MSCP10, Lonza) were thawed and seeded in a 384-well plate (Thermo Scientific™ 164688) in Williams E medium (Gibco™ A1217601) supplemented with Primary Hepatocyte Thawing and Plating Supplements (Gibco™ CM3000). Cells were treated with siRNAs at 25 nM using 0.15 μL of Lipofectamine per well or with 100 nM of GalNAc-siRNAs for free-uptake.

### Duplex RT-qPCR

Cells were processed for RT-qPCR read-out using the Cells-to-CT 1-step TaqMan Kit (Invitrogen™ A25603). Briefly, cells were washed with 50 μL ice-cold PBS and lysed in 20 μl Lysis solution containing DNase I. Lysis was stopped after 5 minutes by addition of 2 μl STOP Solution for 2 min. For the RT-qPCR analysis, 1 μL of lysate was dispensed per well into a 96-well PCR plate in a 20 μL RT-qPCR reaction volume. RT-qPCR was performed using the TaqMan® 1-Step qRT-PCR Mix from the Cells-to-CT 1-step TaqMan Kit, with TaqMan probes for GAPDH (VIC_PL, Assay Id Hs00266705_g1) and PCSK9 (FAM, Assay Id Hs00545399-m1) or ApoB (FAM, Assay Id Mm01545150_m1). RT-qPCR was performed using a QuantStudio 5 thermocycling instrument (Applied BioSystems). Relative quantification was determined using the ΔΔCT method, where GAPDH was used as internal control and expression changes normalized to the reference sample (no siRNA treatment).

### In Vivo Mouse Study Animals

Male C57BL/6J mice (20-25 g) were group housed in the Saretius animal unit at the University of Reading, and maintained under a 12 h light/dark cycle, at 23° C. with humidity controlled according to Home Office regulations. Mice were given access to standard rodent chow SDS rat expanded diet (RM3-E-FG) for the duration of the study.

Formulation of siRNA Compounds

Compound A, Compound G, and Compound H were each formulated in RNAase free PBS to concentrations of 0.4 and 2 mg/mL, to provide doses of 2 and 10 mg/kg when given subcutaneously (SC) in a 5 mL/kg dosing volume. Control groups (n=5) received Vehicle (RNase-free PBS) SC at 5 mL/kg dosing volume.

### Liver Processing for RT-qPCR

At Day 2 (48 hrs) and Day 7 (168 hrs) following siRNA compound or Vehicle injection (n=5), each treatment group was terminally sampled by cardiac puncture under isoflurane. Liver tissue was excised and snap frozen in liquid N₂ Total RNA was extracted from homogenates of snap-frozen whole liver using GenElute™ Total RNA Purification Kit (RNB100-100RXN) Duplex RT-qPCR was performed using the ThermoFisher TaqMan Fast 1-Step Master Mix with TaqMan probes for GAPDH (VIC_PL), PCSK9 (FAM) and mTTR (FAM). Relative quantification (RQ) of PCSK9 was determined using the ΔΔCT method, where GAPDH was used as internal control and the expression changes of the target gene were normalized to the vehicle control.

### Example 1

### Testing 5′ Versus 3′ Positioning of Crook on the Sense Strand (SS) of Unmodified ‘Inclisiran’ Sequence in Serum Stability Assays

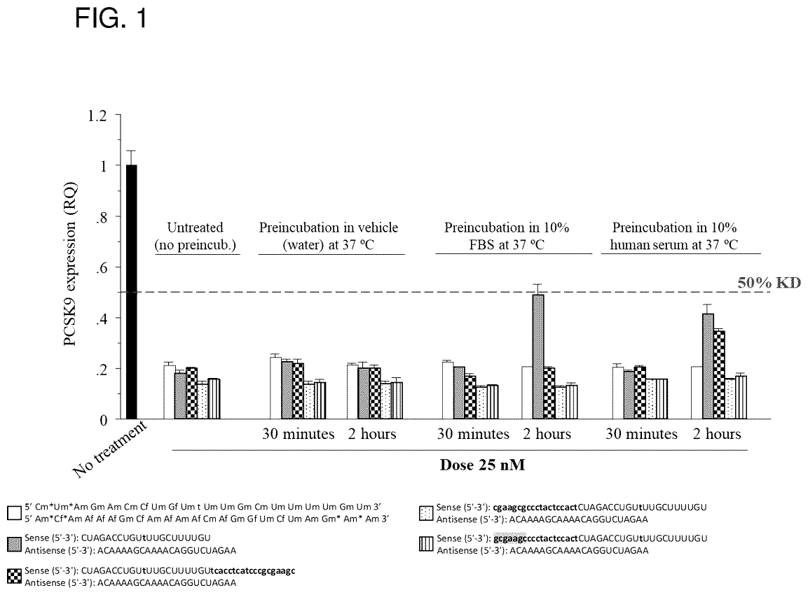

Following a 2 hr incubation in 10% FBS or 10% human serum, unmodified ‘Inclisiran’ with Crook positioned either at the 5′ or 3′ end of the SS, shows increased target mRNA (PCSK9) knockdown (KD) compared to the ‘no crook’ siRNA. However, superior KD is observed when crook is on the 5′ end compared to 3′ end of SS, following pre-treatment in human serum. This is demonstrated in FIG. 1, where 5′ SS crook siRNA [striped bar] containing hairpin sequence GCGAAGC, maintains high levels of target KD (85%) in HepG2 cells following 2 hr treatment with 10% human serum comparable to that observed with modified Inclisiran (80%) [white bar]. Similar results can be shown when ‘reversed’ crook hairpin (CGAAGCG) is placed at the 5′ end of the SS [spotted bar]. In contrast, 3′ SS positioned Crook [hatched bar] shows −18.75% (loss of target KD) in HepG2 cells following pre-treatment in human serum; 65% KD (compared to 80% KD with no serum incubation). As expected, unmodified ‘Inclisiran’ with no Crook attached [grey bar] shows reduced levels of target KD after pre-treatment in either FBS or human serum: 50% and 60% KD, respectively, equating to −26.8% and −39% loss of KD.

### Example 2

Testing 5′ Positioning of Crook on the Sense Strand (SS) of Unmodified ‘Incilsiran’ Sequence in Serum Stability Assays with Increasing Concentrations of FBS

Following 2 hr incubations at 37 C in increasing concentrations of FBS, unmodified ‘Inclisiran’ sequence with Crook positioned at the 5′ end of the SS [striped bar] shows sustained target mRNA (PCSK9) knockdown (70-80% KD) in all concentrations of FBS tested (10%, 20% and 50%), comparable to levels observed with modified Inclisiran (70-80% KD) [white bar]. Similarly, ‘reversed’ crook hairpin (CGAAGCG) on the 5′ end of SS provides 65-75% KD with no loss of KD [spotted bar]. In contrast, the ‘no crook’ compound [grey bar] displays up to −85% loss of KD as only 20-50% target KD, is evident following serum treatment.

### Example 3

### Testing 5′ Positioning of Crook on the Sense Strand (SS) of Unmodified ‘Inclisiran’ Sequence in Serum Stability Assays Over a 4-Hr Incubation Period

After a 4 hr incubation in either 10% FBS or 10% human serum, unmodified ‘Inclisiran’ with Crook positioned at the 5′ end of the SS, shows sustained levels of approx. 75% target mRNA (PCSK9) knockdown (KD), and 65% KD, respectively [striped bar]. Similarly, there is no loss of KD evident for ‘reversed’ crook hairpin (CGAAGCG) on the 5′ end of SS [spotted bar], comparable with modified ‘Inclisiran’, where approximately 70% KD is observed [white bar]. In contrast, the absence of Crook [grey bar] leads to substantially lower levels of KD following 4 hr pre-treatment in 10% FBS (45% KD) or 10% human serum (35% KD), equating to a −36% and −50% loss in KD, respectively.

### Example 4

Testing 5′ Versus 3′ Positioning of Crook on an Unmodified siRNA Sequence Targeting Pcsk9, in Serum Stability Assays (Sequence Termed PC8-18)

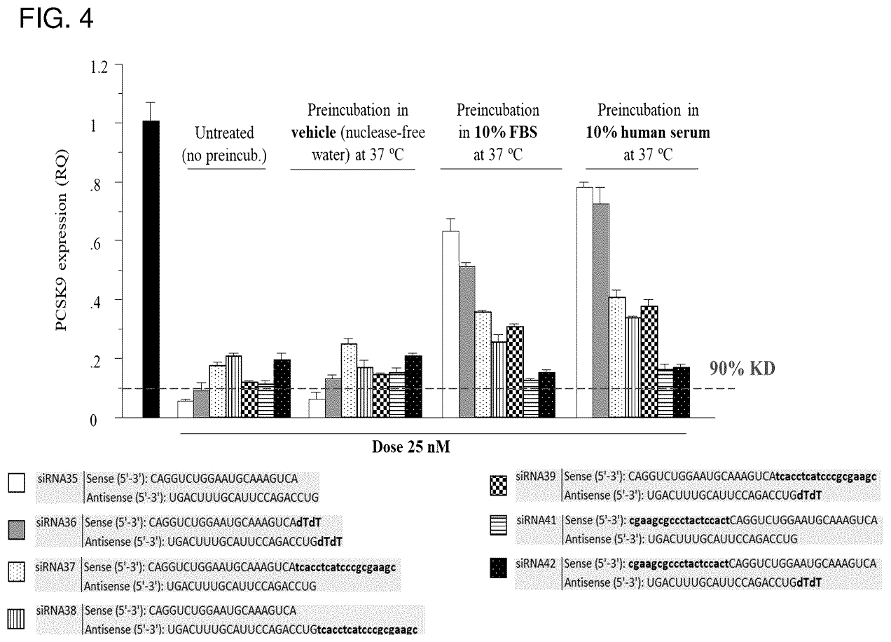

Following a 2 hr incubation in 10% FBS or 10% human serum, PC8-18 with Crook positioned at the 5′ end of the sense strand (SS), shows superior levels of knockdown (KD) of target mRNA (PCSK9) compared to 3′ positioned Crook on either the SS or AS. This is shown in FIG. 4, where there is sustained target KD (approx. 85%) for PC8-18 siRNA with 5′SS Crook: [horizontal striped bar & spots on black background bar] compared to 60-70% KD (equating to a loss of 30% KD compared to no serum treatment) seen with 3′ SS positioned Crook [spots on white background bar & hatched bar]. Similarly, when Crook is placed on 3′ AS, loss of KD is 6-16% resulting in 65-75% target KD [vertical striped bar]. When Crook is not present on PC8-18 siRNA, target KD is reduced to only 35% following 2 hr incubation in FBS equating to a substantial loss of KD (−63% compared to no serum treatment), and to only 25% KD in human serum (−77%). Similarly, uncrooked molecules that contain 3′ dTdT overhangs, show loss of KD levels of −44% and −72% (compared to no serum treatment) following pre-treatment in FBS and human serum, respectively.

### Example 5 Testing the In Vivo Silencing Effect of 5′ Versus 3′ Positioning of Crook on an Unmodified siRNA Compound Targeting PCSK9 (PC2 Sequence)

Groups of 5 mice for each treatment group were injected subcutaneously (SC) with either vehicle (PBS), compound A (no Crook), compound G (Crook on 5′ end of sense strand (SS)), or compound H (Crook on 3′ end of SS). Each compound was given at either 2 mg/kg or 10 mg/kg, and following sacrifice, levels of liver PCSK9 mRNA were measured at two time points (day 2 and day 7).

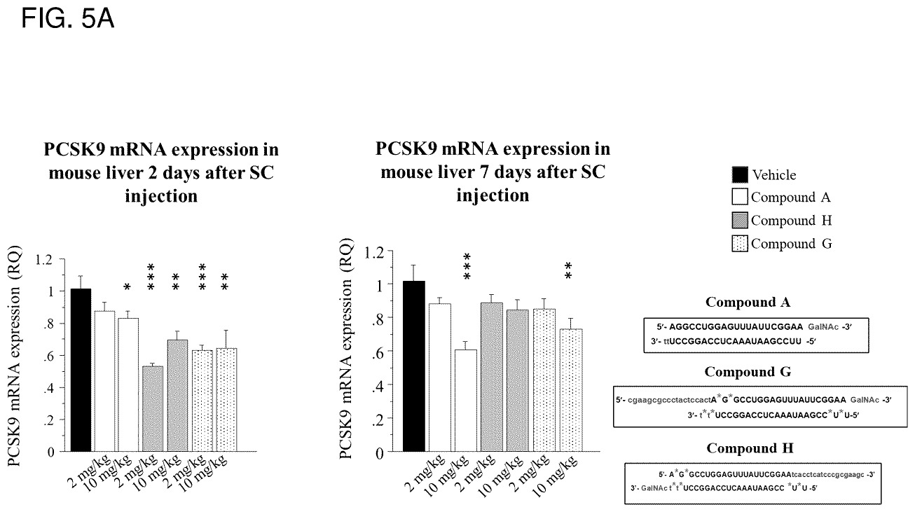

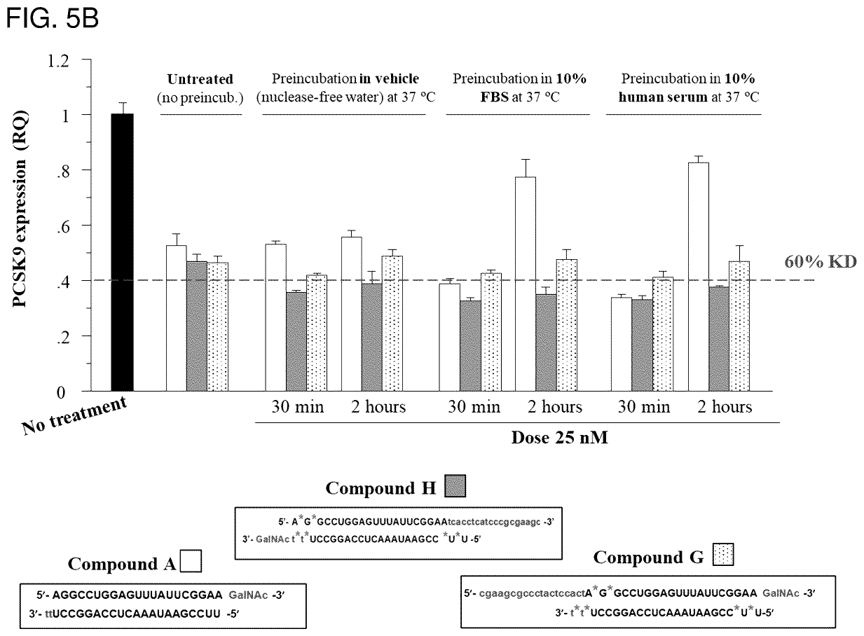

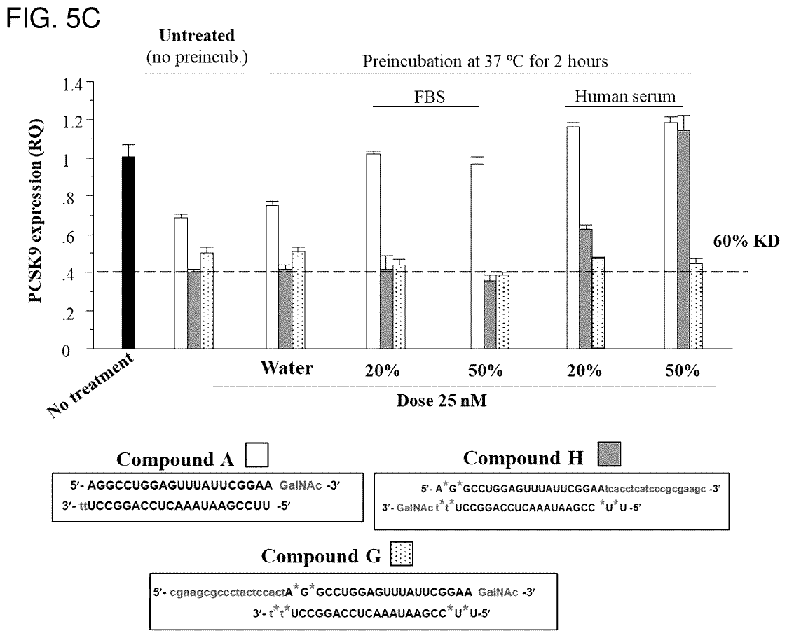

Compound G (5′ SS Crook) results in 40% KD of PCSK9 mRNA in the liver after 48 hours at 2 and 10 mg/kg and 30% KD at 10 mg/kg after 7 days, compared to vehicle controls (FIG. 5A). Comparable liver target KD is seen after 48 hrs for compound H (3′ SS Crook) approx. 50% KD at 2 mg/kg (30% KD at 10 mg/kg), with no significant KD observed at day 7 (FIG. 5A). Compound A which contains no Crook, shows noticeably less target KD, with no silencing following SC injection of 2 mg/kg dose at either 2 or 7 days. At the 10 mg/kg dose, compound A shows and <20% KD after 48 hrs, and 40% after 7 days (FIG. 5A).

### Example 6

### Testing Compounds a, G and H in Serum Stability Assays (HepG2 Cells)

Comparable results are shown for both 5′ and 3′ positioned Crook on the SS. Compound G (5′ SS Crook) and compound H (3′ SS Crook) maintains PCSK9 mRNA KD of >50% following a 2 hr incubation in either 10% FBS or human serum (compared to no serum treatment). In contrast, there is loss of target KD seen for compound A (no Crook), from 50% to only 20% KD following a 2 hr serum treatment; FIG. 5B.

When these siRNA compounds were further challenged in increasing serum concentrations (20% and 50%) over a 2 hr period, compound G (5′SS Crook) displayed superior performance over 3′SS positioned Crook (H) in human serum. This is shown in FIG. 5C, where a sustained level of target mRNA KD (approx. 50%) is evident only in compound G [spotted bar] following 2 hrs incubation in 50% human serum. This equates to no loss of KD for G when compared to its ‘no serum’ treatment KD level. In contrast, compound H [grey bar] shows a complete loss in KD (0%) performing exactly as ‘no crook’ compound A [white bar] after 2 hrs in 50% human serum.

TABLE 1
Selection of Lp(a) candidate siRNA sequences to which crook is conjugated

| siRNA ID | | | |
|---|---|---|---|
| (SEQ ID | (SEQ ID | | |
| NO) | NO) | Sense sequence (5′-3′) | Antisense sequence (5′-3′) |
| LP1 (43) | LP9 (2) | GCCCCUUAUUGUUAUACG | CGUAUAACAAUAAGGGGC |
| | | (SEQ ID NO: 43) | (SEQ ID NO: 2) |
| LP2 (44) | LP10 (3) | GCCCCUUAUUGUUAUACGA | UCGUAUAACAAUAAGGGGC |
| | | (SEQ ID NO: 44) | (SEQ ID NO: 3) |
| LP3 (45) | LP11 (4) | GCCCCUUAUUGUUAUACGA | TCGUAUAACAAUAAGGGGC |
| | | (SEQ ID NO: 45) | (SEQ ID NO: 4) |
| LP4 (46) | LP12 (5) | CCCCUUAUUGUUAUACGA | UCGUAUAACAAUAAGGGG |
| | | (SEQ ID NO: 46) | (SEQ ID NO: 5) |
| LPS (47) | LP13 (6) | CCCUUAUUGUUAUACGA | UCGUAUAACAAUAAGGG |
| | | (SEQ ID NO: 47) | (SEQ ID NO: 6) |
| LP6 (48) | LP14 (7) | CCCCUUAUUGUUAUACA | UGUAUAACAAUAAGGGG |
| | | (SEQ ID NO: 48) | (SEQ ID NO: 7) |
| LP7 (49) | LP15 (41) | CGGUAAUGGACAGAGUUAU | AUAACUCUGUCCAUUACCG |
| | | (SEQ ID NO: 49) | (SEQ ID NO: 41) |
| LP8 (42) | LP16 (35) | ACAGCCCCUUAUUGUUAUACGA | CGUAUAACAAUAAGGGGC |
| | | (SEQ ID NO: 42) | (SEQ ID NO: 35) |

TABLE 2
Selection of APOC III and DGAT 2 siRNA sequences
to which crook is conjugated

| SEQ | | |
|---|---|---|
| ID | | |
| NO | Name | Sequence |
| 50 | APOC3_01 | 5′-ACGGGACAGUAUUCUCAGUNA |
| 51 | APOC3_02 | 5′-CCCAAUAAAGCUGGACAAGAA |
| 52 | APOC3_03 | 5′-CUGUAGGUUGCUUAAAAGGGA |
| 53 | APOC3_04 | 5′-CUGGAGCACCGUUAAGGACAA |
| 54 | APOC3_05 | 5′-UCCCAAUAAAGCUGGACAAGA |
| 55 | APOC3_06 | 5′-GCCCCUGUAGGUUGCUUAAAA |
| 56 | APOC3_07 | 5′-CCCUGAAAGACUACUGGAGCA |
| 57 | APOC3_08 | 5′-UGCUUAAAAGGGACAGUAUUC |
| 58 | APOC3_09 | 5′-GACCUCAAUACCCCAAGUCCA |
| 59 | APOC3_10 | 5′-GAGCACCGUUAAGGACAAGUU |
| 60 | APOC3_01 | 5′-ACGGGACAGUAUUCUCAGUNA |
| 61 | APOC3_02 | 5′-CCCAAUAAAGCUGGACAAGAA |
| 62 | APOC3_03 | 5′-CUGUAGGUUGCUUAAAAGGGA |
| 63 | APOC3_04 | 5′-CUGGAGCACCGUUAAGGACAA |
| 64 | APOC3_05 | 5′-UCCCAAUAAAGCUGGACAAGA |
| 65 | APOC3_06 | 5′-GCCCCUGUAGGUUGCUUAAAA |
| 66 | APOC3_07 | 5′-CCCUGAAAGACUACUGGAGCA |
| 67 | APOC3_08 | 5′-UGCUUAAAAGGGACAGUAUUC |
| 68 | APOC3_09 | 5′-GACCUCAAUACCCCAAGUCCA |
| 69 | APOC3_10 | 5′-GAGCACCGUUAAGGACAAGUUt |
| 70 | APOC3_01 | 5′-UCACUGAGAAUACUGUCCCGU-3′ |
| 71 | APOC3_02 | 5′-UUCUUGUCCAGCUUUAUUGGG-3′ |
| 72 | APOC3_03 | 5′-UCCCUUUUAAGCAACCUACAG-3′ |
| 73 | APOC3_04 | 5′-UUGUCCUUAACGGUGCUCCAG-3′ |
| 74 | APOC3_05 | 5′-UCUUGUCCAGCUUUAUUGGGA-3′ |
| 75 | APOC3_06 | 5′-UUUUAAGCAACCUACAGGGGC-3′ |
| 76 | APOC3_07 | 5′-UGCUCCAGUAGUCUUUCAGGG-3′ |
| 77 | APOC3_08 | 5′-GAAUACUGUCCCUUUUAAGCA-3′ |
| 78 | APOC3_09 | 5′-UGGACUUGGGGUAUUGAGGUC-3′ |
| 79 | APOC3_10 | 5′-AACUUGUCCUUAACGGUGCUC-3′ |
| 80 | APOC3_01 | 5′-UCACUGAGAAUACUGUCCCGU |
| 81 | APOC3_02 | 5′-UUCUUGUCCAGCUUUAUUGGG |
| 82 | APOC3_03 | 5′-UCCCUUUUAAGCAACCUACAG |
| 83 | APOC3_04 | 5′-UUGUCCUUAACGGUGCUCCAG |
| 84 | APOC3_05 | 5′-UCUUGUCCAGCUUUAUUGGGA |
| 85 | APOC3_06 | 5′-UUUUAAGCAACCUACAGGGGC |
| 86 | APOC3_07 | 5′-UGCUCCAGUAGUCUUUCAGGG |
| 87 | APOC3_08 | 5′-GAAUACUGUCCCUUUUAAGCA |
| 88 | APOC3_09 | 5′-UGGACUUGGGGUAUUGAGGUC |
| 89 | APOC3_10 | 5′-AACUUGUCCUUAACGGUGCUC |
| 90 | DGAT2_01 | 5′-CUCUGUAAAUUUGGAAGUGUC |
| 91 | DGAT2_02 | 5′-CACCAUGAGCUAGGUGGAGUA |
| 92 | DGAT2_03 | 5′-UUCCUGAAGUGACAAAGGAAA |
| 93 | DGAT2_04 | 5′-GACCACCAGGAACUAUAUCUU |
| 94 | DGAT2_05 | 5′-GUUCCAGAAAUACAUUGGUUU |
| 95 | DGAT2_06 | 5′-AACCGCAAGGGCUUUGUGAAA |
| 96 | DGAT2_07 | 5′-GAGCAAGAAGUUCCCAGGCAU |
| 97 | DGAT2_08 | 5′-CAGUAGUAGGCAUCUGGAAUG |
| 98 | DGAT2_09 | 5′-GUCAUGGGUGUCUGUGGGUUA |
| 99 | DGAT2_10 | 5′-GCUCUGUAAAUUUGGAAGUGU |
| 100 | DGAT2_01 | 5′-CUCUGUAAAUUUGGAAGUGUC |
| 101 | DGAT2_02 | 5′-CACCAUGAGCUAGGUGGAGUA |
| 102 | DGAT2_03 | 5′-UUCCUGAAGUGACAAAGGAAA |
| 103 | DGAT2_04 | 5′-GACCACCAGGAACUAUAUCUU |
| 104 | DGAT2_05 | 5′-GUUCCAGAAAUACAUUGGUUU |
| 105 | DGAT2_06 | 5′-AACCGCAAGGGCUUUGUGAAA |
| 106 | DGAT2_07 | 5′-GAGCAAGAAGUUCCCAGGCAU |
| 107 | DGAT2_08 | 5′-CAGUAGUAGGCAUCUGGAAUG |
| 108 | DGAT2_09 | 5′-GUCAUGGGUGUCUGUGGGUUA |
| 109 | DGAT2_10 | 5′-GCUCUGUAAAUUUGGAAGUGU |
| 110 | DGAT2_01 | 5′-GACACUUCCAAAUUUACAGAG-3′ |
| 111 | DGAT2_02 | 5′-UACUCCACCUAGCUCAUGGUG-3′ |
| 112 | DGAT2_03 | 5′-UUUCCUUUGUCACUUCAGGAA-3′ |
| 113 | DGAT2_04 | 5′-AAGAUAUAGUUCCUGGUGGUC-3′ |
| 114 | DGAT2_05 | 5′-AAACCAAUGUAUUUCUGGAAC-3′ |
| 115 | DGAT2_06 | 5′-UUUCACAAAGCCCUUGCGGUU-3′ |
| 116 | DGAT2_07 | 5′-AUGCCUGGGAACUUCUUGCUC-3′ |
| 117 | DGAT2_08 | 5′-CAUUCCAGAUGCCUACUACUG-3′ |
| 118 | DGAT2_09 | 5′-UAACCCACAGACACCCAUGAC-3′ |
| 119 | DGAT2_10 | 5′-ACACUUCCAAAUUUACAGAGC-3′ |
| 120 | DGAT2_01 | 5′-GACACUUCCAAAUUUACAGAG |
| 121 | DGAT2_02 | 5′-UACUCCACCUAGCUCAUGGUG |
| 122 | DGAT2_03 | 5′-UUUCCUUUGUCACUUCAGGAA |
| 123 | DGAT2_04 | 5′-AAGAUAUAGUUCCUGGUGGUC |
| 124 | DGAT2_05 | 5′-AAACCAAUGUAUUUCUGGAAC |
| 125 | DGAT2_06 | 5′-UUUCACAAAGCCCUUGCGGUU |
| 126 | DGAT2_07 | 5′-AUGCCUGGGAACUUCUUGCUC |
| 127 | DGAT2_08 | 5′-CAUUCCAGAUGCCUACUACUG |
| 128 | DGAT2_09 | 5′-UAACCCACAGACACCCAUGAC |
| 129 | DGAT2_10 | 5 -ACACUUCCAAAUUUACAGAGC |

TABLE 3
Selection of DGAT2 siRNA sequences (SEQ ID NOs 131-170), PCSK9
(SEQ ID NO: 171-210 and ApoCIII (SEQ ID NO: 211 to 250)

| SEQ ID NO | Sequence | |
|---|---|---|
| 131 | GACCACCAGGAACUAUAUCUU | sense sequence |
| 132 | GUUCCAGAAAUACAUUGGUUU | sense sequence |
| 133 | AACCGCAAGGGCUUUGUGAAA | sense sequence |
| 134 | GAGCAAGAAGUUCCCAGGCAU | sense sequence |
| 135 | CUUUGGAGAGAAUGAAGUGUA | sense sequence |
| 136 | CUUCGACAAGCACAAGACCAA | sense sequence |
| 137 | GCCGAUGGGUCCAGAAGAAGU | sense sequence |
| 138 | CUUCACUUGGCUGGUGUUUGA | sense sequence |
| 139 | CUCCUUUGGAGAGAAUGAAGU | sense sequence |
| 140 | UGCCAUCCUCAUGUACAUAUU | sense sequence |
| 141 | CCGCAAGGGCUUUGUGAAACU | sense sequence |
| 142 | AGCAAGAAGUUCCCAGGCAUA | sense sequence |
| 143 | AGUGUACAAGCAGGUGAUCUU | sense sequence |
| 144 | UGCUGACCACCAGGAACUAUA | sense sequence |
| 145 | CCGAUGGGUCCAGAAGAAGUU | sense sequence |
| 146 | UUUGGAGAGAAUGAAGUGUAC | sense sequence |
| 147 | UGGCGCUACUUUCGAGACUAC | sense sequence |
| 148 | AAUGCCUGUGUUGAGGGAGUA | sense sequence |
| 149 | AGUUCCAGAAAUACAUUGGUU | sense sequence |
| 150 | CAGAAGUGAGCAAGAAGUUCC | sense sequence |
| 151 | AAGAUAUAGUUCCUGGUGGUC | antisense sequence |
| 152 | AAACCAAUGUAUUUCUGGAAC | antisense sequence |
| 153 | UUUCACAAAGCCCUUGCGGUU | antisense sequence |
| 154 | AUGCCUGGGAACUUCUUGCUC | antisense sequence |
| 155 | UACACUUCAUUCUCUCCAAAG | antisense sequence |
| 156 | UUGGUCUUGUGCUUGUCGAAG | antisense sequence |
| 157 | ACUUCUUCUGGACCCAUCGGC | antisense sequence |
| 158 | UCAAACACCAGCCAAGUGAAG | antisense sequence |
| 159 | ACUUCAUUCUCUCCAAAGGAG | antisense sequence |
| 160 | AAUAUGUACAUGAGGAUGGCA | antisense sequence |
| 161 | AGUUUCACAAAGCCCUUGCGG | antisense sequence |
| 162 | UAUGCCUGGGAACUUCUUGCU | antisense sequence |
| 163 | AAGAUCACCUGCUUGUACACU | antisense sequence |
| 164 | UAUAGUUCCUGGUGGUCAGCA | antisense sequence |
| 165 | AACUUCUUCUGGACCCAUCGG | antisense sequence |
| 166 | GUACACUUCAUUCUCUCCAAA | antisense sequence |
| 167 | GUAGUCUCGAAAGUAGCGCCA | antisense sequence |
| 168 | UACUCCCUCAACACAGGCAUU | antisense sequence |
| 169 | AACCAAUGUAUUUCUGGAACU | antisense sequence |
| 170 | GGAACUUCUUGCUCACUUCUG | antisense sequence |
| 171 | CCUCAUAGGCCUGGAGUUUAU | sense sequence |
| 172 | AGGCCUGGAGUUUAUUCGGAA | sense sequence |
| 173 | CCCUCAUAGGCCUGGAGUUUA | sense sequence |
| 174 | AGGUCUGGAAUGCAAAGUCAA | sense sequence |
| 175 | GGCCUGGAGUUUAUUCGGAAA | sense sequence |
| 176 | CAGGUCUGGAAUGCAAAGUCA | sense sequence |
| 177 | CCUCACCAAGAUCCUGCAUGU | sense sequence |
| 178 | ACCCUCAUAGGCCUGGAGUUU | sense sequence |
| 179 | CACCAGCAUACAGAGUGACCA | sense sequence |
| 180 | AUCUCCUAGACACCAGCAUAC | sense sequence |
| 181 | UCCUAGACACCAGCAUACAGA | sense sequence |
| 182 | CUGGAGUUUAUUCGGAAAAGC | sense sequence |
| 183 | GCCUGGAGUUUAUUCGGAAAA | sense sequence |
| 184 | GAGGCAGAGACUGAUCCACUU | sense sequence |
| 185 | UAGGCCUGGAGUUUAUUCGGA | sense sequence |
| 186 | CACUUCUCUGCCAAAGAUGUC | sense sequence |
| 187 | AUGCAAAGUCAAGGAGCAUGG | sense sequence |
| 188 | GGUCAUGGUCACCGACUUCGA | sense sequence |
| 189 | GGCAGCUGUUUUGCAGGACUG | sense sequence |
| 190 | GGGCAGGUUGGCAGCUGUUUU | sense sequence |
| 191 | AUAAACUCCAGGCCUAUGAGG | antisense sequence |
| 192 | UUCCGAAUAAACUCCAGGCCU | antisense sequence |
| 193 | UAAACUCCAGGCCUAUGAGGG | antisense sequence |
| 194 | UUGACUUUGCAUUCCAGACCU | antisense sequence |
| 195 | UUUCCGAAUAAACUCCAGGCC | antisense sequence |
| 196 | UGACUUUGCAUUCCAGACCUG | antisense sequence |
| 197 | ACAUGCAGGAUCUUGGUGAGG | antisense sequence |
| 198 | AAACUCCAGGCCUAUGAGGGU | antisense sequence |
| 199 | UGGUCACUCUGUAUGCUGGUG | antisense sequence |
| 200 | GUAUGCUGGUGUCUAGGAGAU | antisense sequence |
| 201 | UCUGUAUGCUGGUGUCUAGGA | antisense sequence |
| 202 | GCUUUUCCGAAUAAACUCCAG | antisense sequence |
| 203 | UUUUCCGAAUAAACUCCAGGC | antisense sequence |
| 204 | AAGUGGAUCAGUCUCUGCCUC | antisense sequence |
| 205 | UCCGAAUAAACUCCAGGCCUA | antisense sequence |
| 206 | GACAUCUUUGGCAGAGAAGUG | antisense sequence |
| 207 | CCAUGCUCCUUGACUUUGCAU | antisense sequence |
| 208 | UCGAAGUCGGUGACCAUGACC | antisense sequence |
| 209 | CAGUCCUGCAAAACAGCUGCC | antisense sequence |
| 210 | AAAACAGCUGCCAACCUGCCC | antisense sequence |
| 211 | CUGGAGCACCGUUAAGGACAA | sense sequence |
| 212 | CCCUGAAAGACUACUGGAGCA | sense sequence |
| 213 | GAGCACCGUUAAGGACAAGUU | sense sequence |
| 214 | ACUGGAGCACCGUUAAGGACA | sense sequence |
| 215 | CCUGAAAGACUACUGGAGCAC | sense sequence |
| 216 | AAGACUACUGGAGCACCGUUA | sense sequence |
| 217 | CAGUUCCCUGAAAGACUACUG | sense sequence |
| 218 | GGUGACCGAUGGCUUCAGUUC | sense sequence |
| 219 | GGGUGACCGAUGGCUUCAGUU | sense sequence |
| 220 | ACUACUGGAGCACCGUUAAGG | sense sequence |
| 221 | GACUACUGGAGCACCGUUAAG | sense sequence |
| 222 | UUCAGUUCCCUGAAAGACUAC | sense sequence |
| 223 | GUUCCCUGAAAGACUACUGGA | sense sequence |
| 224 | UGGAGCACCGUUAAGGACAAG | sense sequence |
| 225 | CGCCACCAAGACCGCCAAGGA | sense sequence |
| 226 | GGGCUGGGUGACCGAUGGCUU | sense sequence |
| 227 | GCCACCAAGACCGCCAAGGAU | sense sequence |
| 228 | AGACUACUGGAGCACCGUUAA | sense sequence |
| 229 | CCACCAAGACCGCCAAGGAUG | sense sequence |
| 230 | UCCCUGAAAGACUACUGGAGC | sense sequence |
| 231 | UUGUCCUUAACGGUGCUCCAG | antisense sequence |
| 232 | UGCUCCAGUAGUCUUUCAGGG | antisense sequence |
| 233 | AACUUGUCCUUAACGGUGCUC | antisense sequence |
| 234 | UGUCCUUAACGGUGCUCCAGU | antisense sequence |
| 235 | GUGCUCCAGUAGUCUUUCAGG | antisense sequence |
| 236 | UAACGGUGCUCCAGUAGUCUU | antisense sequence |
| 237 | CAGUAGUCUUUCAGGGAACUG | antisense sequence |
| 238 | GAACUGAAGCCAUCGGUCACC | antisense sequence |
| 239 | AACUGAAGCCAUCGGUCACCC | antisense sequence |
| 240 | CCUUAACGGUGCUCCAGUAGU | antisense sequence |
| 241 | CUUAACGGUGCUCCAGUAGUC | antisense sequence |
| 242 | GUAGUCUUUCAGGGAACUGAA | antisense sequence |
| 243 | UCCAGUAGUCUUUCAGGGAAC | antisense sequence |
| 244 | CUUGUCCUUAACGGUGCUCCA | antisense sequence |
| 245 | UCCUUGGCGGUCUUGGUGGCG | antisense sequence |
| 246 | AAGCCAUCGGUCACCCAGCCC | antisense sequence |
| 247 | AUCCUUGGCGGUCUUGGUGGC | antisense sequence |
| 248 | UUAACGGUGCUCCAGUAGUCU | antisense sequence |
| 249 | CAUCCUUGGCGGUCUUGGUGG | antisense sequence |
| 250 | GCUCCAGUAGUCUUUCAGGGA | antisense sequence |

TABLE 4
siRNAs pairs used in silencing of APOC3 and DGAT 2 gene expression in
HEPG2 cells in vitro

| Name | Sense | Antisense |
|---|---|---|
| APOC3_ | 5′- | 5′- |
| 01 | ACGGGACAGUAUUCUCAGUNAtcacctcatccc | UCACUGAGAAUACUGUCC |
| | gcgaagc-3′ (SEQ ID NO 401) | CGU-3′ (SEQ ID NO 70) |
| APOC3_ | 5′- | 5′- |
| 02 | CCCAAUAAAGCUGGACAAGAAtcacctcatccc | UUCUUGUCCAGCUUUAUU |
| | gcgaagc-3′(SEQ ID NO 402) | GGG-3′(SEQ ID NO 71) |
| APOC3_ | 5′- | 5′- |
| 03 | CUGUAGGUUGCUUAAAAGGGAtcacctcatccc | UCCCUUUUAAGCAACCUA |
| | gcgaagc-3′(SEQ ID NO 403) | CAG-3(SEQ ID NO 72) |
| APOC3_ | 5′- | 5′- |
| 04 | CUGGAGCACCGUUAAGGACAAtcacctcatccc | UUGUCCUUAACGGUGCU |
| | gcgaagc-3′(SEQ ID NO 404) | CCAG-3′(SEQ ID NO 73) |
| APOC3_ | 5′- | 5′- |
| 05 | UCCCAAUAAAGCUGGACAAGAtcacctcatccc | UCUUGUCCAGCUUUAUUG |
| | gcgaagc-3′(SEQ ID NO 405) | GGA-3′(SEQ ID NO 74) |
| APOC3_ | 5′- | 5′- |
| 06 | GCCCCUGUAGGUUGCUUAAAAtcacctcatccc | UUUUAAGCAACCUACAGG |
| | gcgaagc-3′(SEQ ID NO 406) | GGC-3′(SEQ ID NO 75) |
| APOC3_ | 5′- | 5′- |
| 07 | CCCUGAAAGACUACUGGAGCAtcacctcatccc | UGCUCCAGUAGUCUUUCA |
| | gcgaagc-3′ (SEQ ID NO 407) | GGG-3′(SEQ ID NO 76) |
| APOC3_ | 5′- | 5′- |
| 08 | UGCUUAAAAGGGACAGUAUUCtcacctcatccc | GAAUACUGUCCCUUUUAA |
| | gcgaagc-3′ (SEQ ID NO 408) | GCA-3′(SEQ ID NO 77) |
| APOC3_ | 5′- | 5′- |
| 09 | GACCUCAAUACCCCAAGUCCAtcacctcatccc | UGGACUUGGGGUAUUGA |
| | gcgaagc-3 (SEQ ID NO 409) | GGUC-3′(SEQ ID NO 78) |
| APOC3_ | 5′- | 5′- |
| 10 | GAGCACCGUUAAGGACAAGUUtcacctcatccc | AACUUGUCCUUAACGGUG |
| | gcgaagc-3′(SEQ ID NO 410) | CUC-3′(SEQ ID NO 79) |
| DGAT2_ | 5′- | 5′- |
| 01 | CUCUGUAAAUUUGGAAGUGUCtcacctcatccc | GACACUUCCAAAUUUACA |
| | gcgaagc-3′ (SEQ ID NO 411) | GAG-3′(SEQ ID NO 110) |
| DGAT2_ | 5′- | 5′- |
| 02 | CACCAUGAGCUAGGUGGAGUAtcacctcatccc | UACUCCACCUAGCUCAUG |
| | gcgaagc-3′(SEQ ID NO 412) | GUG-3′(SEQ ID NO 111) |
| DGAT2_ | 5′- | 5′- |
| 03 | UUCCUGAAGUGACAAAGGAAAtcacctcatccc | UUUCCUUUGUCACUUCAG |
| | gcgaagc-3′(SEQ ID NO 413) | GAA-3′(SEQ ID NO 112) |
| DGAT2_ | 5′- | 5′- |
| 04 | GACCACCAGGAACUAUAUCUUtcacctcatccc | AAGAUAUAGUUCCUGGUG |
| | gcgaagc-3 (SEQ ID NO 414) | GUC-3(SEQ ID NO 113) |
| DGAT2_ | 5′- | 5′- |
| 05 | GUUCCAGAAAUACAUUGGUUUtcacctcatccc | AAACCAAUGUAUUUCUGG |
| | gcgaagc-3′(SEQ ID NO 415) | AAC-3′(SEQ ID NO 114) |
| DGAT2_ | 5′- | 5′- |
| 06 | AACCGCAAGGGCUUUGUGAAAtcacctcatccc | UUUCACAAAGCCCUUGCG |
| | gcgaagc-3″(SEQ ID NO 416) | GUU-3′(SEQ ID NO 115) |
| DGAT2_ | 5- | 5′- |
| 07 | GAGCAAGAAGUUCCCAGGCAUtcacctcatccc | AUGCCUGGGAACUUCUU |
| | gcgaagc-3′(SEQ ID NO 417) | GCUC-3′(SEQ ID NO 116) |
| DGAT2_ | 5′- | 5′- |
| 08 | CAGUAGUAGGCAUCUGGAAUGtcacctcatccc | CAUUCCAGAUGCCUACUA |
| | gcgaagc-3′(SEQ ID NO 418) | CUG-3′(SEQ ID NO 117) |
| DGAT2_ | 5′- | 5′- |
| 09 | GUCAUGGGUGUCUGUGGGUUAtcacctcatcc | UAACCCACAGACACCCAU |
| | cgcgaagc-3′(SEQ ID NO 419) | GAC-3′(SEQ ID NO 118) |
| DGAT2_ | 5′- | 5′- |
| 10 | GCUCUGUAAAUUUGGAAGUGUtcacctcatccc | ACACUUCCAAAUUUACAG |
| | gcgaagc-3 (SEQ ID NO 420) | AGC-3′(SEQ ID NO 119) |

TABLE 5
Crook structures tested in the serum stability assay for 5′ crook
siRNAs 14b to siRNA15-5′CR consist of unmodified ‘inclisiran’ sequence (C = crook;
CR = reversed hairpin Crook).
siRNAs 35-44 consist of PC8 sequence
siRNAs A, G and H consist of PC2 sequence

| Oligo name | Sequence |
|---|---|
| siRNA14m | Sense: 5′ Cm*Um*Am Gm Am Cm Cf Um Gf Um t Um Um Gm Cm Um Um Um |
| ′Inclisiran′ | Um Gm Um 3′ (SEQ ID NO 494) |
| | Antisense: 5′ Am*Cf*Am Af Af Af Gm Cf Am Af Am Af Cm Af Gm Gf Um Cf Um |
| | Am Gm* Am* Am 3′ (SEQ ID NO 495) |
| siRNA14b | Sense (5′-3′): CUAGACCUGULUUGCUUUUGU (SEQ ID NO 389) |
| | Antisense (5′-3′): ACAAAAGCAAAACAGGUCUAGAA (SEQ ID NO 390) |
| siRNA15b | Sense (5′-3′): CUAGACCUGUtUUGCUUUUGUtcacctcatcccgcgaagc (SEQ ID NO 496) |
| | Antisense (5′-3′): ACAAAAGCAAAACAGGUCUAGAA (SEQ ID NO 390) |
| siRNA15-5′C | Sense (5′-3′): cgaagcgccctactccactCUAGACCUGUUUGCUUUUGU (SEQ ID NO 497) |
| | Antisense (5′-3′): ACAAAAGCAAAACAGGUCUAGAA(SEQ ID NO 390) |
| siRNA15-5′CR | Sense (5′-3′): gcgaagcccctactccactCUAGACCUGUTUUGCUUUUGU (SEQ ID NO 498) |
| | Antisense (5′-3′): ACAAAAGCAAAACAGGUCUAGAA(SEQ ID NO 390) |
| siRNA35 | Sense (5′-3′): CAGGUCUGGAAUGCAAAGUCA(SEQ ID NO 278 and 262 and 176) |
| | Antisense (5′-3′): UGACUUUGCAUUCCAGACCUG(SEQ ID NO 272, 196, 334) |
| siRNA36 | Sense (5′-3′): CAGGUCUGGAAUGCAAAGUCAdTdT (SEQ ID NO 421) |
| | Antisense (5′-3′): UGACUUUGCAUUCCAGACCUGdTdT (SEQ ID NO 422) |
| siRNA37 | Sense (5′-3′): |
| | CAGGUCUGGAAUGCAAAGUCAdTdCdAdCdCdTdCdAdTdCdCdCdGdCdGdAdAd |
| | GdC (Seq ID NO 423) |
| | Antisense (5′-3′): UGACUUUGCAUUCCAGACCUG (SEQ ID NO 272, 196, 334) |
| siRNA 38 | Sense (5′-3′): CAGGUCUGGAAUGCAAAGUCA (SEQ ID NO 278 and 262 and 176) |
| | Antisense (5′-3′): |
| | UGACUUUGCAUUCCAGACCUGdTdCdAdCdCdTdCdAdTdCdCdCdGdCdGdAdAd |
| | GdC (SEQ ID NO 424) |
| siRNA39 | Sense (5′-3′): |
| | CAGGUCUGGAAUGCAAAGUCAdTdCdAdCdCdTdCdAdTdCdCdCdGdCdGdAdAd |
| | GdC (Seq ID NO 423) |
| | Antisense (5′-3′): UGACUUUGCAUUCCAGACCUGdTdT (SEQ ID NO 422) |
| siRNA40 | Sense (5′-3′): CAGGUCUGGAAUGCAAAGUCAdTdT SEQ ID NO 421) |
| | Antisense (5′-3′): |
| | UGACUUUGCAUUCCAGACCUGdTdCdAdCdCdTdCdAdTdCdCdCdGdCdGdAdAd |
| | GdC (SEQ ID NO 424) |
| siRNA41 | Sense (5′-3′): |
| | dCdGdAdAdGdCdGdCdCdCdTdAdCdTdCdCdAdCdTCAGGUCUGGAAUGCAAAG |
| | UCA (SEQ ID NO 425) |
| | Antisense (5′-3′): UGACUUUGCAUUCCAGACCUG (SEQ ID NO 272, 196, 334) |
| siRNA42 | Sense (5′-3′): |
| | dCdGdAdAdGdCdGdCdCdCdTdAdCdTdCdCdAdCdTCAGGUCUGGAAUGCAAAG |
| | UCA (SEQ ID NO 425) |
| | Antisense (5′-3′): UGACUUUGCAUUCCAGACCUGdTdT (SEQ ID NO 422) |
| siRNA43 | Sense (5′-3′): |
| | dCdGdAdAdGdCdGdCdCdCdTdAdCdTdCdCdAdCdTCAGGUCUGGAAUGCAAAG |
| | UCAdTdT (SEQ ID NO 426) |
| | Antisense (5′-3′): UGACUUUGCAUUCCAGACCUG (SEQ ID NO 272, 196, 334) |
| siRNA44 | Sense (5′-3′): CAGGUCUGGAAUGCAAAGUCA (SEQ ID NO 278 and 262 and 176) |
| | Antisense (5′-3′): |
| | dCdGdAdAdGdCdGdCdCdCdTdAdCdTdCdCdAdCdTUGACUUUGCAUUCCAGAC |
| | CUG (SEQ ID NO 427) |
| siRNA-A | 5′-AGGCCUGGAGUUUAUUCGGAA GalNAc-3′ (SEQ ID NO 172 and 256) |
| | 3′-ttUCCGGACCUCAAAUAAGCCUU-5′ (SEQ ID NO 252 and 254) |
| siRNA-G | 5′-cgaagcgccctactccactA*G*GCCUGGAGUUUAUUCGGAA GalNAc-3′ (SEQ ID |
| | NO 428) |
| | 3′-t*t*UCCGGACCUCAAAUAAGCC*U*U-5′ (SEQ ID NO: 527) |
| siRNA-H | 5′-AGGCCUGGAGUUUAUUCGGAAtcacctcatcccgcgaagc-3′ (SEQ ID NO 253) |
| | 3′-GalNAc UCCGGACCUCAAAUAAGCCUU-5′ (SEQ ID NO 429) |

Legend:
c, g, a, t or dT, dG, dA, dC: DNA bases
A, G, C, U: RNA bases
f: 2′-deoxy-2′-fluoro
m: 2′-O-methyl
*internucleotide linkage phosphorothioate (PS) GalNAc

### Example 7 (Inclisiran, PCSK9 Sequence)

When crook was attached at the 5′ end of the sense strand (siRNA15-5′C), the siRNA sequence maintained a full KD activity against the target PCSK9 comparable to the chemically modified version (siRNA4m) after 2-hour incubation in 10% FBS or human serum. Crook at the 3′ end of the sense strand (siRNA5b) showed partial protection in HS. siRNA with short crook (harpin part only) at the 3′ (siRNA15s7) and 5′ end (Inc 03), as well as the stem 12-nt part only at the 5′ end (INC_02), all showed significant loss of KD compared to the full 19-nt crook when transfected in HepG2 at 25 nM (Table 6 and 7).

TABLE 6

| Sequence | KD in no | KD after | KD after | % KD loss | % KD loss |
|---|---|---|---|---|---|
| name | serum | 10% FBS | 10% HS | in FBS | in HS |
| siRNA14m | 78.7 | 79.6 | 79.9 | 0.0 | 0.0 |
| siRNA14b | 82.0 | 51.4 | 59.1 | 37.3 | 28.0 |
| siRNA15b | 80.1 | 80.6 | 65.9 | 0.0 | 17.6 |
| siRNA15-5′C | 86.5 | 87.6 | 84.7 | 0.0 | 2.0 |
| siRNA15s7 | 80.9 | 44.8 | 51.4 | 44.6 | 36.5 |

TABLE 7

| siRNA name | KD in no serum | KD in 10% HS | % KD loss in HS |
|---|---|---|---|
| siRNA14m | 50.4 | 62.2 | 0.0 |
| siRNA14b | 50.1 | 28.7 | 42.8 |
| siRNA15-5′C | 50.6 | 58.6 | 0.0 |
| INC_02 | 34.2 | 0.0 | 100.0 |
| INC_03 | 44.7 | 0.0 | 100.0 |

TABLE 8
siRNA description

| siRNA name | Description | Sequence |
|---|---|---|
| siRNA14m | Fully chemically | S (5′-3′) Cm*Um*Am Gm Am Cm Cf Um Gf Um t Um Um Gm |
| | modified version | Cm Um Um Um Um Gm Um (SEQ ID NO: 494) |
| | | AS (5′-3′) Am*Cf*Am Af Af Af Gm Cf Am Af Am Af Cm Af Gm |
| | | Gf Um Cf Um Am Gm* Am* Am (SEQ ID NO: 495) |
| siRNA14b | No crook | S (5′-3′): CUAGACCUGUtUUGCUUUUGU (SEQ ID NO: 389) |
| | | AS (5′-3′): ACAAAAGCAAAACAGGUCUAGAA (SEQ ID NO: 390) |
| siRNA15b | Crook on 3′ S | S (5′-3′): CUAGACCUGUtUUGCUUUUGUtcacctcatcccgcgaagc |
| | strand | (SEQ ID NO: 496) |
| | | AS (5′-3′): ACAAAAGCAAAACAGGUCUAGAA (SEQ ID NO: 390) |
| siRNA15-5′C | Crook on 5′ S | S (5′-3′): cgaagcgccctactccactCUAGACCUGUtUUGCUUUUGU |
| | strand | (SEQ ID NO: 497) |
| | | AS (5′-3′): ACAAAAGCAAAACAGGUCUAGAA (SEQ ID NO: 390) |
| siRNA15s7 | Harpin part only | S (5′-3′): CUAGACCUGUtUUGCUUUUGUgcgaagc (SEQ ID NO |
| | on 3′ S strand | 430) |
| | | AS (5′-3′): ACAAAAGCAAAACAGGUCUAGAA (SEQ ID NO 390) |
| Inc_02 | 12-nt stem only, | S (5′-3′): ccctactccactCUAGACCUGUtUUGCUUUUGU (SEQ ID |
| | no hairpin | NO 431) |
| | | AS (5′-3′): ACAAAAGCAAAACAGGUCUAGAA (SEQ ID NO 390) |
| Inc 03 | Hairpin on 5′ S | S (5′-3′): cgaagcgCUAGACCUGUtUUGCUUUUGU (SEQ ID NO |
| | strand | 432) |
| | | AS (5′-3′): ACAAAAGCAAAACAGGUCUAGAA (SEQ ID NO 390) |

### Example 8 (P8-18 PCSK9 Sequence)

When Crook was attached at the 5′ end of the sense strand (PC8_05), the siRNA sequence maintained a full KD activity against the target PCSK9 after 2-hour incubation in 80% HS which was comparable to the level of KD observed with no serum pre-incubation. Crook at the 3′ end of the sense strand (PC8_01) gave substantially reduced protection in HS showing 72% percentage loss of KD compared to no serum pre-incubation. siRNA with short crook (harpin part only) at the 3′ (P8_03) and 5′ end (PC8_11), as well as the stem 12-nt part only at the 5′ end (PC8_10), all showed significant loss of KD compared to the full 19-nt crook when transfected in HepG2 at 25 nM (Table 9).

TABLE 9

| siRNA name | KD in no serum | KD in 80% HS | % KD loss in 80% HS |
|---|---|---|---|
| PC8_00 | 72.9 | 0.0 | 100.0 |
| PC8_01 | 51.6 | 14.5 | 72.0 |
| PC8_03 | 50.0 | 1.4 | 97.3 |
| PC8_05 | 63.3 | 60.4 | 4.6 |
| PC8_10 | 54.0 | 25.8 | 52.2 |
| PC8_11 | 72.6 | 17.3 | 76.2 |

TABLE 10

| siRNA name | Description | Sequence |
|---|---|---|
| PC8_00 | No crook | S (5′-3′): CAGGUCUGGAAUGCAAAGUCA (SEQ ID NO 262, 278, |
| | | 176) |
| | | AS (5′-3′): UGACUUUGCAUUCCAGACCUG (SEQ ID NO 272, 196, |
| | | 334) |
| PC8_01 | Crook on 3′ S | S (5′-3′): CAGGUCUGGAAUGCAAAGUCAtcacctcatcccgcgaagc (SEQ |
| | strand | ID NO 433) |
| | | AS (5′-3′): UGACUUUGCAUUCCAGACCUG (SEQ ID NO 272, 196, |
| | | 334) |
| PC8_03 | Crook hairpin | S (5′-3′): CAGGUCUGGAAUGCAAAGUCAgcgaagc (SEQ ID NO 434) |
| | on 3′ S strand | AS (5′-3′): UGACUUUGCAUUCCAGACCUG (SEQ ID NO 272, 196, |
| | | 334) |
| PC8_05 | Crook on 5′ S | S (5′-3′): cgaagcgccctactccactCAGGUCUGGAAUGCAAAGUCA (SEQ |
| | strand | ID NO 435) |
| | | AS (5′-3′): UGACUUUGCAUUCCAGACCUG (SEQ ID NO 272, 196, |
| | | 334) |
| PC8_10 | 12-nt stem | S (5′-3′): ccctactccactCAGGUCUGGAAUGCAAAGUCA (SEQ ID NO |
| | only, no | 436) |
| | hairpin | AS (5′-3′): UGACUUUGCAUUCCAGACCUG (SEQ ID NO 272, 196, |
| | | 334) |
| SIRNA_A | No crook | S (5′- 3′) AGGCCUGGAGUUUAUUCGGAA GalNAc (SEQ |
| | | ID NO 172, 256) |
| | | AS (3′-5′) ttUCCGGACCUCAAAUAAGCCUU (SEQ ID NO |
| | | 252, 254) |
| SIRNA_G | Crook on 5′ | S (5′-3′) |
| | S strand | cgaagcgccctactccactA*G*GCCUGGAGUUUAUUCGGAA |
| | | GalNAc (SEQ ID NO 437) |
| | | AS (3′-5′) t*t*UCCGGACCUCAAAUAAGCC*U*U (SEQ ID |
| | | NO: 527) |
| SIRNA_H | Crook on 3′ | S (5′-3) |
| | S strand | A*G*GCCUGGAGUUUAUUCGGAAtcacctcatcccgcgaagc |
| | | (SEQ ID NO 438) |
| | | AS (3′-5′) GalNAc t*t*UCCGGACCUCAAAUAAGCC*U*U |
| | | (SEQ ID NO: 528) |
| PC8_11 | Hairpin on 5′ | S (5′-3′): cgaagcgCAGGUCUGGAAUGCAAAGUCA (SEQ ID NO: 526) |
| | S strand | AS (5′-3′): UGACUUUGCAUUCCAGACCUG (SEQ ID NO 272, 196, |
| | | 334) |

### Example 9 (Compound G—PCSK9 Sequence)

When Crook was attached at the 5′ end of the sense strand (siRNA_G), the siRNA sequence maintained a full KD activity against PCSK9 after 8-hour incubation in 80% HS comparable to the level of KD observed with no serum pre-incubation (Table 11). In contrast, siRNA_A (no crook) or siRNA_H (crook at the 3′ end of the sense strand) showed no protection in 80% HS and a loss of % KD of 70.8% and 100% respectively when transfected in HepG2 at 25 nM. In a free-uptake assay, siRNA_G showed better KD levels compared to siRNA_H in primary mouse hepatocytes cultured in 10% FBS and treated for 24, 48 and 72 hours with 100 nM of siRNA (Table 12).

TABLE 11

| siRNA name | KD in no serum | KD in 80% HS | % KD loss in HS |
|---|---|---|---|
| siRNA_A | 53.2 | 15.5 | 70.8 |
| siRNA_G | 59.5 | 58.5 | 1.7 |
| siRNA_H | 57.6 | 0.0 | 100.0 |

TABLE 12
KD levels of PCSK9 following free-uptake of
siRNA_A, siRNA_H and siRNA_G at 100 nM in
primary mouse hepatocytes cultured in 10% FBS

| | % KD | % KD | % KD |
|---|---|---|---|
| Time from treatment | siRNA_A | siRNA_H | siRNA_G |
| 24 hours | 0.0 | 0.0 | 15.0 |
| 48 hours | 5.8 | 17.5 | 50.6 |
| 72 hours | 0.0 | 0.0 | 36.5 |

### Example 10 (ApoB Sequences)

When a total of 11 siRNAs carrying a sequence against mouse ApoB were exposed to 20% and 50% human serum, and subsequently transfected at 25 nM into primary mouse hepatocytes, the siRNA variants carrying 5′ crook on the sense strand showed, overall, a better ability to induce KOD of ApoB after exposure to serum compared to the siRNA variants carrying crook on the 3′ end (Table 13).

TABLE 13

| | | KD | KD | KD | KD % | KD % |
|---|---|---|---|---|---|---|
| | Crook | % no | 20% | 50% | loss in | loss in |
| siRNA Name | position | serum | HS | HS | 20% HS | 50% HS |
| TS3_1 | 5′SS | 70.5 | 55.1 | 0.0 | 21.8 | 100.0 |
| TS3_2 | 3′SS | 75.4 | 0.0 | 0.0 | 100.0 | 100.0 |
| TS3_3 | 3′AS | 73.1 | 0.0 | 0.0 | 100.0 | 100.0 |
| TS4_1 | 5′SS | 64.4 | 9.3 | 8.8 | 85.6 | 86.3 |
| TS4_2 | 3′SS | 63.4 | 10.9 | 0.0 | 82.7 | 100.0 |
| TS4_3 | 3′AS | 67.9 | 0.0 | 0.0 | 100.0 | 100.0 |
| ApoB_C10_1 | 5′SS | 58.0 | 67.3 | 62.3 | 0.0 | 0.0 |
| ApoB_C10_2 | 3′SS | 52.4 | 0.0 | 38.2 | 100.0 | 27.0 |
| ApoB_C10_3 | 3′AS | 56.6 | 56.6 | 18.9 | 0.0 | 66.6 |
| ApoB_C3_1 | 5′SS | 48.1 | 50.9 | 37.6 | 0.0 | 21.7 |
| ApoB_C3_2 | 3′SS | 58.1 | 49.8 | 48.0 | 14.2 | 17.3 |
| ApoB_C3_3 | 3′AS | 54.9 | 41.2 | 0.0 | 25.0 | 100.0 |
| ApoB_C2_1 | 5′SS | 66.8 | 87.7 | 87.6 | 0.0 | 0.0 |
| ApoB_C2_2 | 3′SS | 70.9 | 87.1 | 86.8 | 0.0 | 0.0 |
| ApoB_C2_3 | 3′AS | 79.6 | 87.5 | 87.6 | 0.0 | 0.0 |
| ApoB_DM2_1 | 5′SS | 58.4 | 76.4 | 67.4 | 0.0 | 0.0 |
| ApoB_DM2_2 | 3′SS | 64.0 | 65.4 | 4.3 | 0.0 | 93.3 |
| ApoB_DM2_3 | 3′AS | 67.6 | 55.3 | 5.0 | 18.1 | 92.5 |
| ApoB_DM3_1 | 5′SS | 72.0 | 56.4 | 0.0 | 21.7 | 100.0 |
| ApoB_DM3_2 | 3′SS | 75.4 | 0.0 | 0.0 | 100.0 | 100.0 |
| ApoB_DM3_3 | 3′AS | 82.7 | 0.0 | 0.0 | 100.0 | 100.0 |
| ApoB_DM5_1 | 5′SS | 51.6 | 63.7 | 73.4 | 0.0 | 0.0 |
| ApoB_DM5_2 | 3′SS | 59.5 | 0.0 | 0.0 | 100.0 | 100.0 |
| ApoB_DM5_3 | 3′AS | 55.7 | 74.9 | 78.4 | 0.0 | 0.0 |
| ApoB_DM13_1 | 5′SS | 84.8 | 87.2 | 49.1 | 0.0 | 42.1 |
| ApoB_DM13_2 | 3′SS | 88.6 | 50.3 | 45.6 | 43.3 | 48.6 |
| ApoB_DM13_3 | 3′AS | 87.0 | 41.3 | 23.7 | 52.6 | 72.7 |
| ApoB_DM18_1 | 5′SS | 81.8 | 70.8 | 50.9 | 13.4 | 37.8 |
| ApoB_DM18_2 | 3′SS | 83.4 | 80.0 | 36.2 | 4.1 | 56.6 |
| ApoB_DM18_3 | 3′AS | 86.8 | 32.4 | 7.8 | 62.6 | 91.0 |
| ApoB_DM19_1 | 5′SS | 60.0 | 1.7 | 0.0 | 97.1 | 100.0 |
| ApoB_DM19_2 | 3′SS | 68.6 | 0.0 | 0.0 | 100.0 | 100.0 |
| ApoB_DM19_3 | 3′AS | 74.4 | 0.0 | 0.0 | 100.0 | 100.0 |

ApoB sequences Table 14

| siRNA name | Description | Sequence |
|---|---|---|
| TS3_1 | 5′sense | S (5′-3′): |
| | | cgaagcgccctactccactUAGACUUCCUGAAUAAC*U*A (SEQ |
| | | ID NO 439) |
| | | AS (5′-3′): U*A*GUUAUUCAGGAAGUCUA*U*U (SEQ ID NO |
| | | 440) |
| TS3_2 | 3′sense | S (5′-3′): |
| | | U*A*GACUUCCUGAAUAACUAtcacctcatcccgcgaagc (SEQ |
| | | ID NO 441) |
| | | AS (5′-3′): U*A*GUUAUUCAGGAAGUCUA*U*U (SEQ ID NO |
| | | 440) |
| TS3_3 | 3′antisense | S (5′-3′): U*A*GACUUCCUGAAUAAC*U*A (SEQ ID NO |
| | | 442) |
| | | AS (5′-3′): |
| | | U*A*GUUAUUCAGGAAGUCUA*U*Utcacctcatcccgcgaagc |
| | | (SEQ ID NO 443) |
| TS4_1 | 5′sense | S (5′-3′): |
| | | cgaagcgccctactccactUCAUCACACUGAAUACC*A*A (SEQ ID |
| | | NO 444) |
| | | AS (5′-3′): U*U*GGUAUUCAGUGUGAUGA*U*U (SEQ ID |
| | | NO 445) |
| TS4_2 | 3′sense | S (5′-3′): |
| | | U*C*AUCACACUGAAUACCAAtcacctcatcccgcgaagc (SEQ |
| | | ID NO 446) |
| | | AS (5′-3′): U*U*GGUAUUCAGUGUGAUGA*U*U (SEQ ID |
| | | NO 445) |
| TS4_3 | 3′antisense | S (5′-3′): U*C*AUCACACUGAAUACC*A*A (SEQ ID NO |
| | | 447) |
| | | AS (5′-3′): |
| | | U*U*GGUAUUCAGUGUGAUGA*U*Utcacctcatcccgcgaagc |
| | | (SEQ ID NO 448) |
| ApoB_C10_1 | 5′sense | S (5′-3′): |
| | | cgaagcgccctactccactGUCAUCACACUGAAUACCA*A*U |
| | | (SEQ ID NO 449) |
| | | AS (5′-3′): A*U*UGGUAUUCAGUGUGAUGAC*U*U (SEQ ID |
| | | NO 450) |
| ApoB_C10_2 | 3′sense | S (5′-3′): |
| | | G*U*CAUCACACUGAAUACCAAUtcacctcatcccgcgaagc |
| | | (SEQ ID NO 451) |
| | | AS (5′-3′): A*U*UGGUAUUCAGUGUGAUGAC*U*U (SEQ ID |
| | | NO 450) |
| ApoB_C10_3 | 3′antisense | S (5′-3′): G*U*CAUCACACUGAAUACCA*A*U (SEQ ID NO |
| | | 453) |
| | | AS (5′-3′): |
| | | A*U*UGGUAUUCAGUGUGAUGAC*U*Utcacctcatcccgcgaa |
| | | gc (SEQ ID NO 452) |
| ApoB_C3_1 | 5′sense | S (5′-3′): |
| | | cgaagcgccctactccactGGUGUAUGGCUUCAACCCU*G*A |
| | | (SEQ ID NO 454) AS (5′-3′): |
| | | U*C*AGGGUUGAAGCCAUACACC*U*U (SEQ ID NO 455) |
| ApoB_C3_2 | 3′sense | S (5′-3′): |
| | | G*G*UGUAUGGCUUCAACCCUGAtcacctcatcccgcgaagc |
| | | (SEQ ID NO 456) AS (5′-3′): |
| | | U*C*AGGGUUGAAGCCAUACACC*U*U (SEQ ID NO 455) |
| ApoB_C3_3 | 3′antisense | S (5′-3′): G*G*UGUAUGGCUUCAACCCU*G*A (SEQ ID |
| | | NO 457) AS (5′-3′): |
| | | U*C*AGGGUUGAAGCCAUACACC*U*Utcacctcatcccgcgaa |
| | | gc (SEQ ID NO 458) |
| ApoB_C2_1 | 5′sense | S (5′-3′): |
| | | cgaagcgccctactccactCACCAACUUCUUCCACGAG*U*C |
| | | (SEQ ID NO 459) AS (5′-3′): |
| | | G*A*CUCGUGGAAGAAGUUGGUG*U*U (SEQ ID NO 460) |
| ApoB_C2_2 | 3′sense | S (5′-3′): |
| | | C*A*CCAACUUCUUCCACGAGUCtcacctcatcccgcgaagc |
| | | (SEQ ID NO 461) AS (5′-3′): |
| | | G*A*CUCGUGGAAGAAGUUGGUG*U*U(SEQ ID NO 460) |
| ApoB_C2_3 | 3′antisense | S (5′-3′): C*A*CCAACUUCUUCCACGAG*U*C (SEQ ID |
| | | NO 462) AS (5′-3′): |
| | | G*A*CUCGUGGAAGAAGUUGGUG*U*Utcacctcatcccgcgaa |
| | | gc (SEQ ID NO 463) |
| ApoB_DM2_1 | 5′sense | S (5′-3′): |
| | | cgaagcgccctactccactAGGCAGAGCUAGUGGCA*A*A (SEQ |
| | | ID NO 464) |
| | | AS (5′-3′): U*U*UGCCACUAGCUCUGCCU*U*U (SEQ ID NO |
| | | 465) |
| ApoB_DM2_2 | 3′sense | S (5′-3′): |
| | | A*G*GCAGAGCUAGUGGCAAAtcacctcatcccgcgaagc (SEQ |
| | | ID NO 466) |
| | | AS (5′-3′): U*U*UGCCACUAGCUCUGCCU*U*U (SEQ ID NO |
| | | 465) |
| ApoB_DM2_3 | 3′antisense | S (5′-3′): A*G*GCAGAGCUAGUGGCA*A*A (SEQ ID NO |
| | | 467) |
| | | AS (5′-3′): |
| | | U*U*UGCCACUAGCUCUGCCU*U*Utcacctcatcccgcgaagc |
| | | (SEQ ID NO 468) |
| ApoB_DM3_1 | 5′sense | S (5′-3′): |
| | | cgaagcgccctactccactGAGCAAAUCUCUUCAAU*A*A (SEQ |
| | | ID NO 469) |
| | | AS (5′-3′): U*U*AUUGAAGAGAUUUGCUC*U*U (SEQ ID NO |
| | | 470) |
| ApoB_DM3_2 | 3′sense | S (5′-3′): |
| | | G*A*GCAAAUCUCUUCAAUAAtcacctcatcccgcgaagc (SEQ |
| | | ID NO 471) |
| | | AS (5′-3′): U*U*AUUGAAGAGAUUUGCUC*U*U (SEQ ID NO |
| | | 470) |
| ApoB_DM3_3 | 3′antisense | S (5′-3′): G*A*GCAAAUCUCUUCAAU*A*A (SEQ ID NO 472) |
| | | AS (5′-3′): |
| | | U*U*AUUGAAGAGAUUUGCUC*U*Utcacctcatcccgcgaagc |
| | | (SEQ ID NO 473) |
| ApoB_DM5_1 | 5′sense | S (5′-3′): |
| | | cgaagcgccctactccactCCACAAAUGUCUACAGC*A*A (SEQ |
| | | ID NO 474) AS (5′-3′): U*U*GCUGUAGACAUUUGUGG*U*U |
| | | (SEQ ID NO 475) |
| ApoB_DM5_2 | 3′sense | S (5′-3′): |
| | | C*C*ACAAAUGUCUACAGCAAtcacctcatcccgcgaagc (SEQ |
| | | ID NO 476) |
| | | AS (5′-3′): U*U*GCUGUAGACAUUUGUGG*U*U (SEQ ID NO |
| | | 475) |
| ApoB_DM5_3 | 3′antisense | S (5′-3′): C*C*ACAAAUGUCUACAGC*A*A (SEQ ID NO 477) |
| | | AS (5′-3′): |
| | | U*U*GCUGUAGACAUUUGUGG*U*Utcacctcatcccgcgaagc |
| | | (SEQ ID NO 478) |
| ApoB_DM13_1 | 5′sense | S (5′-3′): |
| | | cgaagcgccctactccactGAAACAGGCUUGAAAGA*A*U (SEQ |
| | | ID NO 479) |
| | | AS (5′-3′): A*U*UCUUUCAAGCCUGUUUC*U*U (SEQ ID NO |
| | | 480) |
| ApoB_DM13_2 | 3′sense | S (5′-3′): |
| | | G*A*AACAGGCUUGAAAGAAUtcacctcatcccgcgaagc (SEQ |
| | | ID NO 481) |
| | | AS (5′-3′): A*U*UCUUUCAAGCCUGUUUC*U*U (SEQ ID NO |
| | | 480) |
| ApoB_DM13_3 | 3′antisense | S (5′-3′): G*A*AACAGGCUUGAAAGA*A*U (SEQ ID NO 482) |
| | | AS (5′-3′): |
| | | A*U*UCUUUCAAGCCUGUUUC*U*Utcacctcatcccgcgaagc |
| | | (SEQ ID NO 483) |
| ApoB_DM18_1 | 5′sense | S (5′-3′): |
| | | cgaagcgccctactccactGAGAGAAAUCGAAGAGG*A*A (SEQ |
| | | ID NO 484) |
| | | AS (5′-3′): U*U*CCUCUUCGAUUUCUCUC*U*U (SEQ ID NO |
| | | 485) |
| ApoB_DM18_2 | 3′sense | S (5′-3′): |
| | | G*A*GAGAAAUCGAAGAGGAAtcacctcatcccgcgaagc (SEQ |
| | | ID NO 486) |
| | | AS (5′-3′): U*U*CCUCUUCGAUUUCUCUC*U*U (SEQ ID NO |
| | | 485) |
| ApoB_DM18_3 | 3′antisense | S (5′-3′): G*A*GAGAAAUCGAAGAGG*A*A (SEQ ID NO 487) |
| | | AS (5′-3′): |
| | | U*U*CCUCUUCGAUUUCUCUC*U*Utcacctcatcccgcgaagc |
| | | (SEQ ID NO 488) |
| ApoB_DM19_1 | 5′sense | S (5′-3′): |
| | | cgaagcgccctactccactAGUUAUAGUCCGUGAGC*U*A (SEQ |
| | | ID NO 489) |
| | | AS (5′-3′): U*A*GCUCACGGACUAUAACU*U*U (SEQ ID NO |
| | | 490) |
| ApoB_DM19_2 | 3′sense | S (5′-3′): |
| | | A*G*UUAUAGUCCGUGAGCUAtcacctcatcccgcgaagc (SEQ |
| | | ID NO 491) |
| | | AS (5′-3′): U*A*GCUCACGGACUAUAACU*U*U (SEQ ID NO |
| | | 490) |
| ApoB_DM19_3 | 3′antisense | S (5′-3′): A*G*UUAUAGUCCGUGAGC*U*A (SEQ ID NO |
| | | 492) |
| | | AS (5′-3′): |
| | | U*A*GCUCACGGACUAUAACU*U*Utcacctcatcccgcgaagc |
| | | (SEQ ID NO 493) |

TABLE 15

| SEQ ID NO | | |
|---|---|---|
| 3 | auaacucugu ccauuaccg | |
| 8 | ucguauaaca auaaggggc | |
| 9 | gauaacucug uccauuacc | |
| 10 | auaacucugu ccauuacca | |
| 11 | uaacucuguc cauuaccgu | |
| 12 | agaaugugcc ucgauaacu | |
| 13 | auaacucugu ccaucacca | |
| 14 | auaacucugu ccaucaccu | |
| 15 | uaacucuguc cauuaccau | |
| 16 | augugccuug auaacucug | |
| 17 | aguuggugcu gcuucagaa | |
| 18 | aauaaggggc ugccacagg | |
| 19 | uaacucuguc caucaccau | |
| 20 | augagccucg auaacucug | |
| 21 | aaugagccuc gauaacucu | |
| 22 | aaugcuucca ggacauuuc | |
| 23 | acaguggugg agaaugugc | |
| 24 | guaugugccu cgauaacuc | |
| 25 | ucgauaacuc uguccauca | |
| 26 | Ugucacugga cauuguguc | |
| 27 | cugggaucca ugguguaac | |
| 28 | agaugaccaa gcuuggcag | |
| 29 | uuaacucugu ccauuaccg | |
| 30 | uuaacucugu ccauuaccc | |
| 31 | uuaacucugu ccauuaccu | |
| 32 | auaacucugu ccauuaccc | |
| 33 | auaacucugu ccauuaccu | |
| 34 | uuaacucugu ccauuacca | |
| 36 | ucguauaaca auaaggggc | |
| 37 | tcguauaaca auaaggggc | |
| 38 | ucguauaaca auaagggg | |
| 39 | ucguauaaca auaaggg | |
| 40 | Uguauaacaa uaagggg | |
| 45 | gccccuuauu guuauacga | |
| 251 | tcacctcatc ccgcgaagc | |
| 255 | ccucauaggc cuggaguuua u | |
| 257 | cccucauagg ccuggaguuu a | |
| 258 | acccucauag gccuggaguu u | |
| 259 | uaggccugga guuuauucgg a | |
| 260 | aggucuggaa ugcaaaguca a | |
| 261 | ggccuggagu uuauucggaa a | |
| 263 | ccucaccaag auccugcaug u | |
| 264 | caccagcaua cagagugacc a | |
| 265 | auaaacucca ggccuaugag g | |
| 266 | uuccgaauaa acuccaggcc u | |
| 267 | uaaacuccag gccuaugagg g | |
| 268 | aaacuccagg ccuaugaggg u | |
| 269 | uccgaauaaa cuccaggccu a | |
| 270 | uugacuuugc auuccagacc u | |
| 271 | uuuccgaaua aacuccaggc c | |
| 273 | acaugcagga ucuuggugag g | |
| 274 | uggucacucu guaugcuggu g | |
| 275 | agcaagcaga cauuuaucuu u | |
| 276 | aggucuggaa ugcaaaguca a | |
| 277 | ggccuggagu uuauucggaa a | |
| 279 | cccaagcaag cagacauuua u | |
| 280 | ccucaccaag auccugcaug u | |
| 281 | uuuucuagac cuguuuugcu u | |
| 282 | acccaagcaa gcagacauuu a | |
| 283 | caccagcaua cagagugacc a | |
| 284 | auucuggguu uuguagcauu u | |
| 285 | aucuccuaga caccagcaua c | |
| 286 | uccuagacac cagcauacag a | |
| 287 | gacauuuauc uuuugggucu g | |
| 288 | uauucugggu uuuguagcau u | |
| 289 | cuggaguuua uucggaaaag c | |
| 290 | gccuggaguu uauucggaaa a | |
| 291 | gaggcagaga cugauccacu u | |
| 292 | aagcaagcag acauuuaucu u | |
| 293 | uagaccuguu uugcuuuugu a | |
| 294 | uuugcuuuug uaacuugaag a | |
| 295 | cacuucucug ccaaagaugu c | |
| 296 | uugcuuuugu aacuugaaga u | |
| 297 | augcasaguc aaggagcaug g | |
| 298 | cccacccaag caagcagaca u | |
| 299 | ggguaacagu gaggcuggga a | |
| 300 | ggucaugguc accgacuucg a | |
| 301 | ggcagcuguu uugcaggacu g | |
| 302 | gggcagguug gcagcuguuu u | |
| 303 | uugaagauau uuauucuggg u | |
| 304 | uggcagcugu uuugcaggac u | |
| 305 | ccggggauac cucaccaaga u | |
| 306 | acugauccac uucucugcca a | |
| 307 | auccacuucu cugccaaaga u | |
| 308 | acuucucugc caaagauguc a | |
| 309 | gucuggaaug caaagucaag g | |
| 310 | cuucucugcc aaagauguca u | |
| 311 | gaguugaggc agagacugau | |
| 312 | gaccuguuuu gcuuuuguaa c | |
| 313 | cggggauacc ucaccaagau c | |
| 314 | uuucuagacc uguuuugcuu u | |
| 315 | ggucuggaau gcaaagucaa g | |
| 316 | uaucuccuag acaccagcau a | |
| 317 | agguuggcag cuguuuugca g | |
| 318 | cuuuucuaga ccuguuuugc u | |
| 319 | cuuuucuaga ccuguuuugc u | |
| 320 | uccacuucuc ugccaaagau g | |
| 321 | uggaguuuau ucggaaaagc c | |
| 322 | ggcagguugg cagcuguuuu g | |
| 323 | uggaggugua ucuccuagac a | |
| 324 | gucaucaaug aggccugguu c | |
| 325 | uucuagaccu guuuugcuuu u | |
| 326 | uucuggguuu uguagcauuu u | |
| 327 | gagacugauc cacuucucug c | |
| 328 | agucaaggag cauggaaucc c | |
| 329 | aucuuuuggg ucuguccucu c | |
| 330 | cacccaagca agcagacauu u | |
| 331 | aaagauaaau gucugcuugc u | |
| 332 | uugacuuugc auuccagacc u | |
| 333 | uuuccgaaua aacuccaggc c | |
| 335 | auaaaugucu gcuugcuugg g | |
| 336 | acaugcagga ucuuggugag g | |
| 337 | aagcaaaaca ggucuagaaa a | |
| 338 | uaaaugucug cuugcuuggg u | |
| 339 | uggucacucu guaugcuggu g | |
| 340 | aaaugcuaca aaacccagaa u | |
| 341 | guaugcuggu gucuaggaga u | |
| 342 | ucuguaugcu ggugucuagg a | |
| 343 | cagacccaaa agauaaaugu c | |
| 344 | aaugcuacaa aacccagaau a | |
| 345 | gcuuuuccga auaaacucca g | |
| 346 | uuuuccgaau aaacuccagg c | |
| 347 | aaguggauca gucucugccu c | |
| 348 | aagauaaaug ucugcuugcu u | |
| 349 | uacaaaagca aaacaggucu a | |
| 350 | ucuucaaguu acaaaagcaa a | |
| 351 | gacaucuuug gcagagaagu g | |
| 352 | aucuucaagu uacaaaagca a | |
| 353 | ccaugcuccu ugacuuugca u | |
| 354 | augucugcuu gcuugggugg g | |
| 355 | uucccagccu cacuguuacc c | |
| 356 | ucgaagucgg ugaccaugac c | |
| 357 | caguccugca aaacagcugc c | |
| 358 | aaaacagcug ccaaccugcc c | |
| 359 | acccagaaua aauaucuuca a | |
| 360 | aguccugcaa aacagcugcc a | |
| 361 | aucuugguga gguauccccg g | |
| 362 | uuggcagaga aguggaucag u | |
| 363 | aucuuuggca gagaagugga u | |
| 364 | ugacaucuuu ggcagagaag u | |
| 365 | ccuugacuuu gcauuccaga c | |
| 366 | augacaucuu uggcagagaa g | |
| 367 | gaucagucuc ugccucaacu c | |
| 368 | guuacaaaag caaaacaggu c | |
| 369 | gaucuuggug agguaucccc g | |
| 370 | aaagcaaaac aggucuagaa a | |
| 371 | cuugacuuug cauuccagac c | |
| 372 | Uaugcuggug ucuaggagau a | |
| 373 | cugcaaaaca gcugccaacc u | |
| 374 | casaacaggu cuagaaaagu u | |
| 375 | agcaaaacag gucuagaaaa g | |
| 376 | caucuuuggc agagaagugg a | |
| 377 | ggcuuuuccg aauaaacucc a | |
| 378 | caaaacagcu gccaaccugc c | |
| 379 | ugucuaggag auacaccucc a | |
| 380 | gaaccaggcc ucauugauga c | |
| 381 | aaaagcaaaa caggucuaga a | |
| 382 | aaaaugcuac aaaacccaga a | |
| 383 | gcagagaagu ggaucagucu c | |
| 384 | gggauuccau gcuccuugac u | |
| 385 | gagaggacag acccaaaaga u | |
| 386 | aaaugucugc uugcuugggu g | |
| 387 | tcacctcatc ccgcgaagc | |
| 388 | agcgacgtcg aggcgctcat ggttgcaggc gggcgccgcc gttcagttca gggtctgagc | 60 |
| | ctggaggagt gagccaggca gtgagactgg ctcgggcggg ccgggacgcg tcgttgcagc | 120 |
| | agcggctccc agctcccagc caggattccg cgcgcccctt cacgcgccct gctcctgaac | 180 |
| | ttcagctcct gcacagtcct ccccaccgca aggctcaagg cgccgccggc gtggaccgcg | 240 |
| | cacggcctct aggtctcctc gccaggacag caacctctcc cctggccctc atgggcaccg | 300 |
| | tcagctccag gcggtcctgg tggccgctgc cactgctgct gctgctgctg ctgctcctgg | 360 |
| | gtcccgcggg cgcccgtgcg caggaggacg aggacggcga ctacgaggag ctggtgctag | 420 |
| | ccttgcgttc cgaggaggac ggcctggccg aagcacccga gcacggaacc acagccacct | 480 |
| | tccaccgctg cgccaaggat ccgtggaggt tgcctggcac ctacgtggtg gtgctgaagg | 540 |
| | aggagaccca cctctcgcag tcagagcgca ctgcccgccg cctgcaggcc caggctgccc | 600 |
| | gccggggata cctcaccaag atcctgcatg tcttccatgg ccttcttcct ggcttcctgg | 660 |
| | tgaagatgag tagcgacctg ctggagctgg ccttgaagtt gccccatgtc gactacatcg | 720 |
| | aggaggactc ctctqtcttt gcccagagca tcccgtggaa cctggagcgg attacccctc | 480 |
| | cacggtaccg ggcggatgaa taccagcccc ccgacggagg cagcctggtg gaggtgtatc | 840 |
| | tcctagacac cagcatacag agtgaccacc gggaaatcga gggcagggtc atggtcaccg | 900 |
| | acttcgagaa tgtgcccgag gaggacggga cccgcttcca cagacaggcc agcaagtgtg | 960 |
| | acagtcatgg cacccacctg gcaggggtgg tcagcggccg ggatgccggc gtggccaagg | 1020 |
| | gtgccagcat gcgcagcctg cgcgtgctca actgccaagg gaagggcacg gttagcggca | 1080 |
| | ccctcatagg cctggagttt attcggaaaa gccagctggt ccagcctgtg gggccactgg | 1140 |
| | tggtgctgct qcccctagcg gatgggtaca gccgcgtcct caacgccgcc tgccagcgcc | 1200 |
| | tggcgagggc tggggtcgtg ctggtcaccg ctgccggcaa cttccgggac gatgcctgcc | 1260 |
| | tctactcccc agcatcagct cccgagctca tcacagttgg ggccaccaat gcccaagacc | 1320 |
| | agccggtgac cctggggact ttggggacca actttggccg ctgtgtggac ctctttgccc | 1380 |
| | caggggagga catcattggt gcctccagcg actgcagcac ctgctttgtg tcacagagtg | 1440 |
| | ggacatcaca ggctgctgcc cacgtggctc gcattgcagc catgatgctc tctgccgagc | 1500 |
| | cggagctcac cctggccgag ttgaggcaga gactgatcca cttctctgcc aaagatgtca | 1560 |
| | tcaatgaggc ctggttccct gaggaccagc gggtactgac ccccaacctg gtggccgccc | 1620 |
| | tgccccccag cacccatggg gcaggttggc agctgttttg caggactgta tggtcagcac | 1680 |
| | actcggggcc tacacggatg gccacagccg tcgcccgctg cgccccagat gaggagctgc | 1740 |
| | tgagctgctc cagtttctcc aggagtggga agcggcgggg cgagcgcatg gaggcccaag | 1800 |
| | ggggcaagct ggtctgccgg gcccacaacg cttttggggg tgagggtgtc tacgccattg | 1860 |
| | ccaggtgctg cctgctaccc caggccaact gcagcgtcca cacagctcca ccagctgagg | 1920 |
| | ccagcatggg gacccgtgtc cactgccacc aacagggcca cgtcctcaca ggctgcagct | 1980 |
| | cccactggga ggtggaggac cttggcaccc acaagccgcc tgtgctgagg ccacgaggtc | 2040 |
| | agcccaacca gtgcgtgggc cacagggagg ccagcatcca cgcttcctgc tgccatgccc | 2100 |
| | caggtctgga atgcaaagtc aaggagcatg gaatcccggc ccctcaggag caggtgaccg | 2160 |
| | tggcctgcga ggagggctgg accctgactg gctgcagtgc cctccctggg acctcccacg | 2220 |
| | tcctgggggc ctacgccgta gacaacacgt gtgtagtcag gagccgggac gtcagcacta | 2280 |
| | caggcagcac cagcgaaggg gccgtgacag ccgttgccat ctgctgccgg agccggcacc | 2340 |
| | tggcgcaggc ctccaaggag ctccagtgac agccccatca caggatgggt gtctggggag | 2400 |
| | ggtcaagggc tagggctgag ctttaaaatg gttccgactt gtccctctct cagccctcca | 2460 |
| | tggcctgqca cgaggggatg gggatgcttc cgcctttccg gggctgctgg cctggccctt | 2520 |
| | gagtggggca gcctccttgc ctggaactca ctcactctgg gtgcctcctc cccaggtgga | 2580 |
| | ggtgccagga agctccctcc ctcactgtgg ggcatttcac cattcaaaca ggtcgagctg | 2640 |
| | tgctcgggtg ctgccagctg ctcccaatgt gccgatgtcc gtgggcagaa tgacttttat | 2700 |
| | tgagctcttg ttccgtgcca ggcattcaat cctcaggtct ccaccaagga ggcaggattc | 2760 |
| | ttcccatgga taggggaggg ggcggtaggg gctgcaggga caaacatcgt tggggggtga | 2820 |
| | gtgtgaaagg tgctgatggc cctcatctcc agctaactgt ggagaagccc ctgggggctc | 2880 |
| | cctgattaat ggaggcttag ctttctggat ggcatctagc cagaggctgg agacaggtgc | 2940 |
| | gcccctggty gtcacaggct gtgccttggt ttcctgagcc acctttactc tgctctatgc | 3000 |
| | caggctgtgc tagcaacacc cacaggtggc ctgcggggag ccatcaccta ggactgactc | 3060 |
| | ggcagtgtgc agtggtgcat gcactgtctc agcraacccg ctccactacc cggcagggta | 3120 |
| | cacattcgca cccctacttc acagaggaag aaacctggaa ccagaggggg cgtgcctgcc | 3180 |
| | aagctcacac agcaggaact gagccagaaa cgcagattgg gctggctctg aagccaagcc | 3240 |
| | tcttcttact tcacccggct gggctcctca tttttacggg taacagtgag gctgggaagg | 3300 |
| | ggaacacaga ccaggaagct cggtgagtga tggcagaacg atgcctgcag gcatggaact | 3360 |
| | ttttccgtta tcacccaggc ctgattcact ggcctggcgg agatgcttct aaggcatggt | 3420 |
| | cgggggagag ggccaacaac tgtccctcct tgagcaccag ccccacccaa gcaagcagac | 3460 |
| | atttatcttt tgggtctgtc ctctctgtty cctttttaca gccaactttt ctagacctgt | 3540 |
| | tttgcttttg taacttgaag atatttattc tgggttttgt agcattttta ttaatatggt | 3600 |
| | gactttttaa aataaaaaca aacaaacgtt gtcctaa | |

| | |
|---|---|
| 391 | gucaucacac ugaauaccaa u |
| 392 | auugguauuc agugugauga cac |
| 393 | uugauguguu uagucgcuau u |
| 394 | uagcgacuaa acacaucaau u |
| 395 | cuacacaaau cagcgauuu |
| 396 | aaaucgcuga uuuguguag |
| 397 | uaaggcuaug aagagauact t |
| 398 | aaguaucucu ucauagccuu a |
| 399 | acaaaagcaa aacaggucua gaa |
| 400 | gcgaagcccc tactccact |
| 427 | cgaagcgccc tactccactu gacuuugcau uccagaccug |
| 473 | uuauugaaga gauuugcucu utcacctcat cccgcgaagc |
| 474 | cgaagcgccc tactccactc cacaaauguc uacagcaa |
| 475 | uugcuguaga cauuuguggu u |
| 499 | GUCAUCACACUGAAUACCA*A*U |
| 500 | G*U*CAUCACACUGAAUACCAAU |
| 501 | A*U*UGGUAUUCAGUGUGAUGAC*U*U |
| 502 | GGUGUAUGGCUUCAACCCU*G*A |
| 503 | G*G*UGUAUGGCUUCAACCCUGA |
| 504 | U*C*AGGGUUGAAGCCAUACACC*U*U |
| 505 | CACCAACUUCUUCCACGAG*U*C |
| 506 | C*A*CCAACUUCUUCCACGAGUC |
| 507 | G*A*CUCGUGGAAGAAGUUGGUG*U*U |
| 508 | AGGCAGAGCUAGUGGCA*A*A |
| 509 | A*G*GCAGAGCUAGUGGCAAA |
| 510 | U*U*UGCCACUAGCUCUGCCU*U*U |
| 511 | GAGCAAAUCUCUUCAAU*A*A |
| 512 | G*A*GCAAAUCUCUUCAAUAA |
| 513 | U*U*AUUGAAGAGAUUUGCUC*U*U |
| 514 | CCACAAAUGUCUACAGC*A*A |
| 515 | C*C*ACAAAUGUCUACAGCAA( |
| 516 | U*U*GCUGUAGACAUUUGUGG*U*U |
| 517 | GAAACAGGCUUGAAAGA*A*U |
| 518 | G*A*AACAGGCUUGAAAGAAU |
| 519 | A*U*UCUUUCAAGCCUGUUUC*U*U |
| 520 | GAGAGAAAUCGAAGAGG*A*A |
| 521 | G*A*GAGAAAUCGAAGAGGAA |
| 522 | U*U*CCUCUUCGAUUUCUCUC*U*U |
| 523 | AGUUAUAGUCCGUGAGC*U*A |
| 524 | A*G*UUAUAGUCCGUGAGCUA |
| 525 | U*A*GCUCACGGACUAUAACU*U*U |

### REFERENCES

Nair, J. K., Willoughby, J. L., Chan, A., Charisse, K., Alam, M. R., Wang, Q., Hoekstra, M., Kandasamy, P., Kel'in, A. V., Milstein, S. and Taneja, N., 2014. Multivalent N-acetylgalactosamine-conjugated siRNA localizes in hepatocytes and elicits robust RNAi-mediated gene silencing. Journal of the American Chemical Society, 136(49), pp. 16958-16961.
Soutschek, J., Akinc, A., Bramlage, B., Charisse, K., Constien, R., Donoghue, M., Elbashir, S., Geick, A., Hadwiger, P., Harborth, J. and John, M., 2004. Therapeutic silencing of an endogenous gene by systemic administration of modified siRNAs. Nature. 432(7014), p. 173

1. A nucleic acid molecule comprising:
a first part that comprises a double stranded inhibitory ribonucleic acid (RNA) molecule comprising a sense strand and an antisense strand; and
a second part that comprises a single stranded deoxyribonucleic acid (DNA) molecule, wherein the 3′ end of said single stranded DNA molecule is covalently linked to the 5′ end of the sense strand of the double stranded inhibitory RNA molecule or wherein the 3′ end of the single stranded DNA molecule is covalently linked to the 5′ of the antisense strand of the double stranded inhibitory RNA molecule, characterized in that the double stranded inhibitory RNA comprises a sense nucleotide sequence that encodes a part of a cardiovascular gene target associated with cardiovascular disease, or a polymorphic sequence variant thereof, and wherein said single stranded DNA molecule comprises a nucleotide sequence that is adapted over at least part of its length to anneal by complementary base pairing to a part of said single stranded DNA to form a double stranded DNA structure comprising a stem and a loop domain, characterized in that said nucleic acid molecule comprises N-acetylgalactosamine and said double stranded inhibitory RNA consists of natural nucleotides.

2-3. (canceled)

4. The nucleic acid molecule according to claim 1, wherein said loop domain comprises the nucleotide sequence GCGAAGC.

5. The nucleic acid molecule according to claim 4 wherein said single stranded DNA molecule comprises the nucleotide sequence selected from the group:
(SEQ ID NO 387 and 251)
5′ TCACCTCATCCCGCGAAGC 3′;
(SEQ ID NO 130)
5′ CGAAGCGCCCTACTCCACT 3′.;
and
(SEQ ID NO 400)
5′ GCGAAGCCCCTACTCCACT 3′.

6. The nucleic acid molecule according to claim 1, wherein:
said inhibitory RNA molecule comprises a two-nucleotide overhang comprising or consisting of at least one deoxythymidine dinucleotide (dTdT);
said sense and/or said antisense strands comprises at least one internucleotide phosphorothioate linkages; and/or
said nucleic acid molecule comprises a vinylphosphonate modification.

7-8. (canceled)

9. The nucleic acid molecule according to claim 1, wherein said double stranded inhibitory RNA molecule is between 17 and 29 nucleotides or 19 to 21 nucleotides in length.

10. The nucleic acid molecule according to claim 1, wherein said cardiovascular gene target is human Lipoprotein (a).

11. The nucleic acid molecule according to claim 10, wherein said double stranded inhibitory RNA molecule comprises an antisense nucleotide sequence selected from the group consisting of: SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 and 34.

12. The nucleic acid molecule according to claim 10, wherein;
said double stranded inhibitory RNA molecule comprises an antisense nucleotide sequence comprising SEQ ID NO: 41 and a sense nucleotide sequence comprising SEQ ID NO: 49, wherein said single stranded DNA molecule is covalently linked to the 5′ end of the sense strand of the double stranded inhibitory RNA molecule;
said double stranded inhibitory RNA molecule comprises an antisense nucleotide sequence comprising SEQ ID NO: 4 and a sense nucleotide sequence comprising SEQ ID NO: 44, wherein said single stranded DNA molecule is covalently linked to the 5′ end of the antisense strand of the double stranded inhibitory RNA molecule; or
said double stranded inhibitory RNA molecule comprises an antisense nucleotide sequence comprising SEQ ID NO: 5 and a sense nucleotide sequence comprising SEQ ID NO: 46, wherein said single stranded DNA molecule is covalently linked to the 5′ end of the antisense strand of the double stranded inhibitory RNA molecule.

13-14. (canceled)

15. The nucleic acid molecule according to claim 1, wherein said cardiovascular gene target is human Apolipoprotein C III (Apo C III).

16. The nucleic acid molecule according to claim 15, wherein;
said nucleic acid molecule comprises an RNA strand comprising a nucleotide sequence selected from the group consisting of: SEQ ID NO: 60, 61, 62, 63, 64, 65, 66, 67, 68, 69, 70, 71, 72, 73, 74, 75, 76, 77, 78 and 79;
said nucleic acid molecule comprises an RNA strand comprising a nucleotide sequence selected from the group consisting of: SEQ ID NO: 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249 and 250; or
said nucleic acid molecule comprises an RNA strand comprising a nucleotide sequence selected from the group consisting of: SEQ ID NO: 50, 51, 52, 53, 54, 55, 56, 57, 58, 80, 81, 82, 83, 84, 85, 86, 87, 88 and 89.

17-18. (canceled)

19. The nucleic acid molecule according to claim 1, wherein said cardiovascular gene target is human diglyceride acyltransferase 2 (DGAT2).

20. The nucleic acid molecule according to claim 19, wherein;
said nucleic acid comprises an RNA strand comprising a nucleotide sequence selected from the group consisting of: SEQ ID NO: 100, 101, 102, 103, 104, 105, 106, 107, 108, 109, 110, 111, 112, 113, 114, 115, 116, 117, 118 and 119;
said nucleic acid comprises an RNA strand comprising a nucleotide sequence selected from the group consisting of: SEQ ID NO: 131, 132, 133, 134, 135, 136, 137, 138, 139, 140, 141, 142, 143, 144, 145, 146, 147, 148, 149, 150, 151, 152, 153, 154, 156, 157, 158, 159, 160, 161, 162, 163, 164, 165, 166, 167, 168, 169 and 170: or
said nucleic acid comprises an RNA strand comprising a nucleotide sequence selected from the group consisting of: SEQ ID NO: 90, 91, 92, 93, 94, 95, 96, 97, 98, 99, 120, 121, 122, 123, 124, 125, 126, 127, 128 and 129.

21-22. (canceled)

23. The nucleic acid molecule according to claim 1, wherein said cardiovascular gene target is human PCSK9.

24. The nucleic acid molecule according to claim 23, wherein;
said nucleic acid molecule comprises an RNA strand comprising a nucleotide sequence selected from the group consisting of: SEQ ID NO: 171, 172, 173, 174, 175, 176, 177, 178, 179, 180, 181, 182, 183, 184, 185, 186, 187, 189, 200, 201, 202, 203, 204, 205, 206, 207, 208, 209 and 210; or
said nucleic acid molecule comprises an RNA strand comprising a nucleotide sequence selected from the group consisting of: SEQ ID NO: 191, 192, 193, 194, 195, 196, 197, 198, 199, 200, 201, 202, 203, 204, 205, 206, 207, 208, 209 and 210.

25. (canceled)

26. The nucleic acid molecule according to claim 1, wherein said cardiovascular gene target is human Apolipoprotein B.

27. The nucleic acid molecule according to claim 26, wherein;
said nucleic acid molecule comprises an RNA strand comprising a nucleotide sequence selected from the group consisting of: SEQ ID NO: 499, 500, 453, 502, 503, 457, 505, 506, 462, 508, 509, 467, 511, 512, 472, 514, 515, 477, 517 518, 482, 520, 521, 487, 523, 524 and 492; or
said nucleic acid molecule comprises an RNA strand comprising a nucleotide sequence selected from the group consisting of: SEQ ID NO: 450, 501, 455, 504, 460, 507, 465, 510, 470, 513, 475, 516, 480, 519, 485, 522, 490 and 525.

28. (canceled)

29. The nucleic acid molecule according to claim 1, wherein said nucleic acid molecule is covalently linked to N-acetylgalactosamine.

30. A pharmaceutical composition comprising at least one nucleic acid molecule according to claim 1 and a pharmaceutically acceptable carrier or excipient.

31. The pharmaceutical composition according to claim 30 wherein said composition comprises at least one further therapeutic agent.

32. A method of treating a subject that has or is predisposed to hypercholesterolemia or diseases associated with hypercholesterolemia, comprising administering to the subject an effective dose of the nucleic acid molecule according to claim 1.

33. The nucleic acid molecule according to claim 1, wherein the 3′ end of said single stranded DNA molecule is covalently linked to the 5′ end of the sense strand of the double stranded inhibitory RNA molecule and said N-acetylgalactosamine is linked to the 3′ end of said sense strand.


SEQUENCE LISTING (528 sequences)

SEQ ID NO: 1 - RNA, 19 nt, synthetic construct
  misc_feature 1..19: note = inhibitory RNA
  source 1..19: mol_type = other RNA
ataactctgtccattaccg

SEQ ID NO: 2 - RNA, 18 nt, synthetic construct
  misc_feature 1..18: note = inhibitory RNA
  source 1..18: mol_type = other RNA
cgtataacaataaggggc

SEQ ID NO: 3 - RNA, 19 nt, synthetic construct
  misc_feature 1..19: note = inhibitory RNA
  source 1..19: mol_type = other RNA
tcgtataacaataaggggc

SEQ ID NO: 4 - RNA, 19 nt, synthetic construct
  modified_base 1: mod_base = OTHER; note = thymine
  misc_feature 1..19: note = inhibitory RNA
  source 1..19: mol_type = other RNA
tcgtataacaataaggggc

SEQ ID NO: 5 - RNA, 18 nt, synthetic construct
  misc_feature 1..18: note = inhibitory RNA
  source 1..18: mol_type = other RNA
tcgtataacaataagggg

SEQ ID NO: 6 - RNA, 17 nt, synthetic construct
  misc_feature 1..17: note = inhibitory RNA
  source 1..17: mol_type = other RNA
tcgtataacaataaggg

SEQ ID NO: 7 - RNA, 17 nt, synthetic construct
  misc_feature 1..17: note = inhibitory RNA
  source 1..17: mol_type = other RNA
tgtataacaataagggg

SEQ ID NO: 8 - RNA, 19 nt, synthetic construct
  misc_feature 1..19: note = inhibitory RNA
  source 1..19: mol_type = other RNA
tcgtataacaataaggggc

SEQ ID NO: 9 - RNA, 19 nt, synthetic construct
  misc_feature 1..19: note = inhibitory RNA
  source 1..19: mol_type = other RNA
gataactctgtccattacc

SEQ ID NO: 10 - RNA, 19 nt, synthetic construct
  misc_feature 1..19: note = inhibitory RNA
  source 1..19: mol_type = other RNA
ataactctgtccattacca

SEQ ID NO: 11 - RNA, 19 nt, synthetic construct
  misc_feature 1..19: note = inhibitory RNA
  source 1..19: mol_type = other RNA
taactctgtccattaccgt

SEQ ID NO: 12 - RNA, 19 nt, synthetic construct
  misc_feature 1..19: note = inhibitory RNA
  source 1..19: mol_type = other RNA
agaatgtgcctcgataact

SEQ ID NO: 13 - RNA, 19 nt, synthetic construct
  misc_feature 1..19: note = inhibitory RNA
  source 1..19: mol_type = other RNA
ataactctgtccatcacca

SEQ ID NO: 14 - RNA, 19 nt, synthetic construct
  misc_feature 1..19: note = inhibitory RNA
  source 1..19: mol_type = other RNA
ataactctgtccatcacct

SEQ ID NO: 15 - RNA, 19 nt, synthetic construct
  misc_feature 1..19: note = inhibitory RNA
  source 1..19: mol_type = other RNA
taactctgtccattaccat

SEQ ID NO: 16 - RNA, 19 nt, synthetic construct
  misc_feature 1..19: note = inhibitory RNA
  source 1..19: mol_type = other RNA
atgtgccttgataactctg

SEQ ID NO: 17 - RNA, 19 nt, synthetic construct
  misc_feature 1..19: note = inhibitory RNA
  source 1..19: mol_type = other RNA
agttggtgctgcttcagaa

SEQ ID NO: 18 - RNA, 19 nt, synthetic construct
  misc_feature 1..19: note = inhibitory RNA
  source 1..19: mol_type = other RNA
aataaggggctgccacagg

SEQ ID NO: 19 - RNA, 19 nt, synthetic construct
  misc_feature 1..19: note = inhibitory RNA
  source 1..19: mol_type = other RNA
taactctgtccatcaccat

SEQ ID NO: 20 - RNA, 19 nt, synthetic construct
  misc_feature 1..19: note = inhibitory RNA
  source 1..19: mol_type = other RNA
atgagcctcgataactctg

SEQ ID NO: 21 - RNA, 19 nt, synthetic construct
  misc_feature 1..19: note = inhibitory RNA
  source 1..19: mol_type = other RNA
aatgagcctcgataactct

SEQ ID NO: 22 - RNA, 19 nt, synthetic construct
  misc_feature 1..19: note = inhibitory RNA
  source 1..19: mol_type = other RNA
aatgcttccaggacatttc

SEQ ID NO: 23 - RNA, 19 nt, synthetic construct
  misc_feature 1..19: note = inhibitory RNA
  source 1..19: mol_type = other RNA
acagtggtggagaatgtgc

SEQ ID NO: 24 - RNA, 19 nt, synthetic construct
  misc_feature 1..19: note = inhibitory RNA
  source 1..19: mol_type = other RNA
gtatgtgcctcgataactc

SEQ ID NO: 25 - RNA, 19 nt, synthetic construct
  misc_feature 1..19: note = inhibitory RNA
  source 1..19: mol_type = other RNA
tcgataactctgtccatca

SEQ ID NO: 26 - RNA, 19 nt, synthetic construct
  misc_feature 1..19: note = inhibitory RNA
  source 1..19: mol_type = other RNA
tgtcactggacattgtgtc

SEQ ID NO: 27 - RNA, 19 nt, synthetic construct
  misc_feature 1..19: note = inhibitory RNA
  source 1..19: mol_type = other RNA
ctgggatccatggtgtaac

SEQ ID NO: 28 - RNA, 19 nt, synthetic construct
  misc_feature 1..19: note = inhibitory RNA
  source 1..19: mol_type = other RNA
agatgaccaagcttggcag

SEQ ID NO: 29 - RNA, 19 nt, synthetic construct
  misc_feature 1..19: note = inhibitory RNA
  source 1..19: mol_type = other RNA
ttaactctgtccattaccg

SEQ ID NO: 30 - RNA, 19 nt, synthetic construct
  misc_feature 1..19: note = inhibitory RNA
  source 1..19: mol_type = other RNA
ttaactctgtccattaccc

SEQ ID NO: 31 - RNA, 19 nt, synthetic construct
  misc_feature 1..19: note = inhibitory RNA
  source 1..19: mol_type = other RNA
ttaactctgtccattacct

SEQ ID NO: 32 - RNA, 19 nt, synthetic construct
  misc_feature 1..19: note = inhibitory RNA
  source 1..19: mol_type = other RNA
ataactctgtccattaccc

SEQ ID NO: 33 - RNA, 19 nt, synthetic construct
  misc_feature 1..19: note = inhibitory RNA
  source 1..19: mol_type = other RNA
ataactctgtccattacct

SEQ ID NO: 34 - RNA, 19 nt, synthetic construct
  misc_feature 1..19: note = inhibitory RNA
  source 1..19: mol_type = other RNA
ttaactctgtccattacca

SEQ ID NO: 35 - RNA, 18 nt, synthetic construct
  misc_feature 1..18: note = passenger sequence
  source 1..18: mol_type = other RNA
cgtataacaataaggggc

SEQ ID NO: 36 - RNA, 19 nt, synthetic construct
  misc_feature 1..19: note = passenger sequence
  source 1..19: mol_type = other RNA
tcgtataacaataaggggc

SEQ ID NO: 37 - RNA, 19 nt, synthetic construct
  modified_base 1: mod_base = OTHER; note = thymine
  misc_feature 1..19: note = passenger sequence
  source 1..19: mol_type = other RNA
tcgtataacaataaggggc

SEQ ID NO: 38 - RNA, 18 nt, synthetic construct
  misc_feature 1..18: note = passenger sequence
  source 1..18: mol_type = other RNA
tcgtataacaataagggg

SEQ ID NO: 39 - RNA, 17 nt, synthetic construct
  misc_feature 1..17: note = passenger sequence
  source 1..17: mol_type = other RNA
tcgtataacaataaggg

SEQ ID NO: 40 - RNA, 17 nt, synthetic construct
  misc_feature 1..17: note = passenger sequence
  source 1..17: mol_type = other RNA
tgtataacaataagggg

SEQ ID NO: 41 - RNA, 19 nt, synthetic construct
  misc_feature 1..19: note = passenger sequence
  source 1..19: mol_type = other RNA
ataactctgtccattaccg

SEQ ID NO: 42 - RNA, 22 nt, synthetic construct
  misc_feature 1..22: note = passenger sequence
  source 1..22: mol_type = other RNA
acagccccttattgttatacga

SEQ ID NO: 43 - RNA, 18 nt, synthetic construct
  misc_feature 1..18: note = passenger sequence
  source 1..18: mol_type = other RNA
gccccttattgttatacg

SEQ ID NO: 44 - RNA, 19 nt, synthetic construct
  misc_feature 1..19: note = passenger sequence
  source 1..19: mol_type = other RNA
gccccttattgttatacga

SEQ ID NO: 45 - RNA, 19 nt, synthetic construct
  misc_feature 1..19: note = passenger sequence
  source 1..19: mol_type = other RNA
gccccttattgttatacga

SEQ ID NO: 46 - RNA, 18 nt, synthetic construct
  misc_feature 1..18: note = passenger sequence
  source 1..18: mol_type = other RNA
ccccttattgttatacga

SEQ ID NO: 47 - RNA, 17 nt, synthetic construct
  misc_feature 1..17: note = passenger sequence
  source 1..17: mol_type = other RNA
cccttattgttatacga

SEQ ID NO: 48 - RNA, 17 nt, synthetic construct
  misc_feature 1..17: note = passenger sequence
  source 1..17: mol_type = other RNA
ccccttattgttataca

SEQ ID NO: 49 - RNA, 19 nt, synthetic construct
  misc_feature 1..19: note = passenger sequence
  source 1..19: mol_type = other RNA
cggtaatggacagagttat

SEQ ID NO: 50 - RNA, 21 nt, synthetic construct
  misc_feature 1..21: note = APOC3_01
  misc_feature 20: note = n is a, c, g, or u
  source 1..21: mol_type = other RNA
acgggacagtattctcagtna

SEQ ID NO: 51 - RNA, 21 nt, synthetic construct
  misc_feature 1..21: note = APOC3_02
  source 1..21: mol_type = other RNA
cccaataaagctggacaagaa

SEQ ID NO: 52 - RNA, 21 nt, synthetic construct
  misc_feature 1..21: note = APOC3_03
  source 1..21: mol_type = other RNA
ctgtaggttgcttaaaaggga

SEQ ID NO: 53 - RNA, 21 nt, synthetic construct
  misc_feature 1..21: note = APOC3_04
  source 1..21: mol_type = other RNA
ctggagcaccgttaaggacaa

SEQ ID NO: 54 - RNA, 21 nt, synthetic construct
  misc_feature 1..21: note = APOC3_05
  source 1..21: mol_type = other RNA
tcccaataaagctggacaaga

SEQ ID NO: 55 - RNA, 21 nt, synthetic construct
  misc_feature 1..21: note = APOC3_06
  source 1..21: mol_type = other RNA
gcccctgtaggttgcttaaaa

SEQ ID NO: 56 - RNA, 21 nt, synthetic construct
  misc_feature 1..21: note = APOC3_07
  source 1..21: mol_type = other RNA
ccctgaaagactactggagca

SEQ ID NO: 57 - RNA, 21 nt, synthetic construct
  misc_feature 1..21: note = APOC3_08
  source 1..21: mol_type = other RNA
tgcttaaaagggacagtattc

SEQ ID NO: 58 - RNA, 21 nt, synthetic construct
  misc_feature 1..21: note = APOC3_09
  source 1..21: mol_type = other RNA
gacctcaataccccaagtcca

SEQ ID NO: 59 - RNA, 21 nt, synthetic construct
  misc_feature 1..21: note = APOC3_10
  source 1..21: mol_type = other RNA
gagcaccgttaaggacaagtt

SEQ ID NO: 60 - RNA, 21 nt, synthetic construct
  misc_feature 1..21: note = inhibitory RNA
  misc_feature 20: note = n is a, c, g, t or u
  source 1..21: mol_type = other RNA
acgggacagtattctcagtna

SEQ ID NO: 61 - RNA, 21 nt, synthetic construct
  misc_feature 1..21: note = inhibitory RNA
  source 1..21: mol_type = other RNA
cccaataaagctggacaagaa

SEQ ID NO: 62 - RNA, 21 nt, synthetic construct
  misc_feature 1..21: note = inhibitory RNA
  source 1..21: mol_type = other RNA
ctgtaggttgcttaaaaggga

SEQ ID NO: 63 - RNA, 21 nt, synthetic construct
  misc_feature 1..21: note = inhibitory RNA
  source 1..21: mol_type = other RNA
ctggagcaccgttaaggacaa

SEQ ID NO: 64 - RNA, 21 nt, synthetic construct
  misc_feature 1..21: note = inhibitory RNA
  source 1..21: mol_type = other RNA
tcccaataaagctggacaaga

SEQ ID NO: 65 - RNA, 21 nt, synthetic construct
  misc_feature 1..21: note = inhibitory RNA
  source 1..21: mol_type = other RNA
gcccctgtaggttgcttaaaa

SEQ ID NO: 66 - RNA, 21 nt, synthetic construct
  misc_feature 1..21: note = inhibitory RNA
  source 1..21: mol_type = other RNA
ccctgaaagactactggagca

SEQ ID NO: 67 - RNA, 21 nt, synthetic construct
  misc_feature 1..21: note = inhibitory RNA
  source 1..21: mol_type = other RNA
tgcttaaaagggacagtattc

SEQ ID NO: 68 - RNA, 21 nt, synthetic construct
  misc_feature 1..21: note = inhibitory RNA
  source 1..21: mol_type = other RNA
gacctcaataccccaagtcca

SEQ ID NO: 69 - RNA, 22 nt, synthetic construct
  modified_base 22: mod_base = OTHER; note = thymine
  misc_feature 1..22: note = inhibitory RNA
  source 1..22: mol_type = other RNA
gagcaccgttaaggacaagttt

SEQ ID NO: 70 - RNA, 21 nt, synthetic construct
  misc_feature 1..21: note = inhibitory RNA
  source 1..21: mol_type = other RNA
tcactgagaatactgtcccgt

SEQ ID NO: 71 - RNA, 21 nt, synthetic construct
  misc_feature 1..21: note = inhibitory RNA
  source 1..21: mol_type = other RNA
ttcttgtccagctttattggg

SEQ ID NO: 72 - RNA, 21 nt, synthetic construct
  misc_feature 1..21: note = inhibitory RNA
  source 1..21: mol_type = other RNA
tcccttttaagcaacctacag

SEQ ID NO: 73 - RNA, 21 nt, synthetic construct
  misc_feature 1..21: note = inhibitory RNA
  source 1..21: mol_type = other RNA
ttgtccttaacggtgctccag

SEQ ID NO: 74 - RNA, 21 nt, synthetic construct
  misc_feature 1..21: note = inhibitory RNA
  source 1..21: mol_type = other RNA
tcttgtccagctttattggga

SEQ ID NO: 75 - RNA, 21 nt, synthetic construct
  misc_feature 1..21: note = inhibitory RNA
  source 1..21: mol_type = other RNA
ttttaagcaacctacaggggc

SEQ ID NO: 76 - RNA, 21 nt, synthetic construct
  misc_feature 1..21: note = inhibitory RNA
  source 1..21: mol_type = other RNA
tgctccagtagtctttcaggg

SEQ ID NO: 77 - RNA, 21 nt, synthetic construct
  misc_feature 1..21: note = inhibitory RNA
  source 1..21: mol_type = other RNA
gaatactgtcccttttaagca

SEQ ID NO: 78 - RNA, 21 nt, synthetic construct
  misc_feature 1..21: note = inhibitory RNA
  source 1..21: mol_type = other RNA
tggacttggggtattgaggtc

SEQ ID NO: 79 - RNA, 21 nt, synthetic construct
  misc_feature 1..21: note = inhibitory RNA
  source 1..21: mol_type = other RNA
aacttgtccttaacggtgctc

SEQ ID NO: 80 - RNA, 21 nt, synthetic construct
  source 1..21: mol_type = other RNA
tcactgagaatactgtcccgt

SEQ ID NO: 81 - RNA, 21 nt, synthetic construct
  source 1..21: mol_type = other RNA
ttcttgtccagctttattggg

SEQ ID NO: 82 - RNA, 21 nt, synthetic construct
  source 1..21: mol_type = other RNA
tcccttttaagcaacctacag

SEQ ID NO: 83 - RNA, 21 nt, synthetic construct
  source 1..21: mol_type = other RNA
ttgtccttaacggtgctccag

SEQ ID NO: 84 - RNA, 21 nt, synthetic construct
  source 1..21: mol_type = other RNA
tcttgtccagctttattggga

SEQ ID NO: 85 - RNA, 21 nt, synthetic construct
  source 1..21: mol_type = other RNA
ttttaagcaacctacaggggc

SEQ ID NO: 86 - RNA, 21 nt, synthetic construct
  source 1..21: mol_type = other RNA
tgctccagtagtctttcaggg

SEQ ID NO: 87 - RNA, 21 nt, synthetic construct
  source 1..21: mol_type = other RNA
gaatactgtcccttttaagca

SEQ ID NO: 88 - RNA, 21 nt, synthetic construct
  source 1..21: mol_type = other RNA
tggacttggggtattgaggtc

SEQ ID NO: 89 - RNA, 21 nt, synthetic construct
  source 1..21: mol_type = other RNA
aacttgtccttaacggtgctc

SEQ ID NO: 90 - RNA, 21 nt, synthetic construct
  misc_feature 1..21: note = antisense or sense RNA
  source 1..21: mol_type = other RNA
ctctgtaaatttggaagtgtc

SEQ ID NO: 91 - RNA, 21 nt, synthetic construct
  misc_feature 1..21: note = antisense or sense RNA
  source 1..21: mol_type = other RNA
caccatgagctaggtggagta

SEQ ID NO: 92 - RNA, 21 nt, synthetic construct
  misc_feature 1..21: note = antisense or sense RNA
  source 1..21: mol_type = other RNA
ttcctgaagtgacaaaggaaa

SEQ ID NO: 93 - RNA, 21 nt, synthetic construct
  misc_feature 1..21: note = antisense or sense RNA
  source 1..21: mol_type = other RNA
gaccaccaggaactatatctt

SEQ ID NO: 94 - RNA, 21 nt, synthetic construct
  misc_feature 1..21: note = antisense or sense RNA
  source 1..21: mol_type = other RNA
gttccagaaatacattggttt

SEQ ID NO: 95 - RNA, 21 nt, synthetic construct
  misc_feature 1..21: note = antisense or sense RNA
  source 1..21: mol_type = other RNA
aaccgcaagggctttgtgaaa

SEQ ID NO: 96 - RNA, 21 nt, synthetic construct
  misc_feature 1..21: note = antisense or sense RNA
  source 1..21: mol_type = other RNA
gagcaagaagttcccaggcat

SEQ ID NO: 97 - RNA, 21 nt, synthetic construct
  misc_feature 1..21: note = antisense or sense RNA
  source 1..21: mol_type = other RNA
cagtagtaggcatctggaatg

SEQ ID NO: 98 - RNA, 21 nt, synthetic construct
  misc_feature 1..21: note = antisense or sense RNA
  source 1..21: mol_type = other RNA
gtcatgggtgtctgtgggtta

SEQ ID NO: 99 - RNA, 21 nt, synthetic construct
  misc_feature 1..21: note = antisense or sense RNA
  source 1..21: mol_type = other RNA
gctctgtaaatttggaagtgt

SEQ ID NO: 100 - RNA, 21 nt, synthetic construct
  misc_feature 1..21: note = inhibitory RNA
  source 1..21: mol_type = other RNA
ctctgtaaatttggaagtgtc

SEQ ID NO: 101 - RNA, 21 nt, synthetic construct
  misc_feature 1..21: note = inhibitory RNA
  source 1..21: mol_type = other RNA
caccatgagctaggtggagta

SEQ ID NO: 102 - RNA, 21 nt, synthetic construct
  misc_feature 1..21: note = inhibitory RNA
  source 1..21: mol_type = other RNA
ttcctgaagtgacaaaggaaa

SEQ ID NO: 103 - RNA, 21 nt, synthetic construct
  misc_feature 1..21: note = inhibitory RNA
  source 1..21: mol_type = other RNA
gaccaccaggaactatatctt

SEQ ID NO: 104 - RNA, 21 nt, synthetic construct
  misc_feature 1..21: note = inhibitory RNA
  source 1..21: mol_type = other RNA
gttccagaaatacattggttt

SEQ ID NO: 105 - RNA, 21 nt, synthetic construct
  misc_feature 1..21: note = inhibitory RNA
  source 1..21: mol_type = other RNA
aaccgcaagggctttgtgaaa

SEQ ID NO: 106 - RNA, 21 nt, synthetic construct
  misc_feature 1..21: note = inhibitory RNA
  source 1..21: mol_type = other RNA
gagcaagaagttcccaggcat

SEQ ID NO: 107 - RNA, 21 nt, synthetic construct
  misc_feature 1..21: note = inhibitory RNA
  source 1..21: mol_type = other RNA
cagtagtaggcatctggaatg

SEQ ID NO: 108 - RNA, 21 nt, synthetic construct
  misc_feature 1..21: note = inhibitory RNA
  source 1..21: mol_type = other RNA
gtcatgggtgtctgtgggtta

SEQ ID NO: 109 - RNA, 21 nt, synthetic construct
  misc_feature 1..21: note = inhibitory RNA
  source 1..21: mol_type = other RNA
gctctgtaaatttggaagtgt

SEQ ID NO: 110 - RNA, 21 nt, synthetic construct
  misc_feature 1..21: note = inhibitory RNA
  source 1..21: mol_type = other RNA
gacacttccaaatttacagag

SEQ ID NO: 111 - RNA, 21 nt, synthetic construct
  misc_feature 1..21: note = inhibitory RNA
  source 1..21: mol_type = other RNA
tactccacctagctcatggtg

SEQ ID NO: 112 - RNA, 21 nt, synthetic construct
  misc_feature 1..21: note = inhibitory RNA
  source 1..21: mol_type = other RNA
tttcctttgtcacttcaggaa

SEQ ID NO: 113 - RNA, 21 nt, synthetic construct
  misc_feature 1..21: note = inhibitory RNA
  source 1..21: mol_type = other RNA
aagatatagttcctggtggtc

SEQ ID NO: 114 - RNA, 21 nt, synthetic construct
  misc_feature 1..21: note = inhibitory RNA
  source 1..21: mol_type = other RNA
aaaccaatgtatttctggaac

SEQ ID NO: 115 - RNA, 21 nt, synthetic construct
  misc_feature 1..21: note = inhibitory RNA
  source 1..21: mol_type = other RNA
tttcacaaagcccttgcggtt

SEQ ID NO: 116 - RNA, 21 nt, synthetic construct
  misc_feature 1..21: note = inhibitory RNA
  source 1..21: mol_type = other RNA
atgcctgggaacttcttgctc

SEQ ID NO: 117 - RNA, 21 nt, synthetic construct
  misc_feature 1..21: note = inhibitory RNA
  source 1..21: mol_type = other RNA
cattccagatgcctactactg

SEQ ID NO: 118 - RNA, 21 nt, synthetic construct
  misc_feature 1..21: note = inhibitory RNA
  source 1..21: mol_type = other RNA
taacccacagacacccatgac

SEQ ID NO: 119 - RNA, 21 nt, synthetic construct
  misc_feature 1..21: note = inhibitory RNA
  source 1..21: mol_type = other RNA
acacttccaaatttacagagc

SEQ ID NO: 120 - RNA, 21 nt, synthetic construct
  source 1..21: mol_type = other RNA
gacacttccaaatttacagag

SEQ ID NO: 121 - RNA, 21 nt, synthetic construct
  source 1..21: mol_type = other RNA
tactccacctagctcatggtg

SEQ ID NO: 122 - RNA, 21 nt, synthetic construct
  source 1..21: mol_type = other RNA
tttcctttgtcacttcaggaa

SEQ ID NO: 123 - RNA, 21 nt, synthetic construct
  source 1..21: mol_type = other RNA
aagatatagttcctggtggtc

SEQ ID NO: 124 - RNA, 21 nt, synthetic construct
  source 1..21: mol_type = other RNA
aaaccaatgtatttctggaac

SEQ ID NO: 125 - RNA, 21 nt, synthetic construct
  source 1..21: mol_type = other RNA
tttcacaaagcccttgcggtt

SEQ ID NO: 126 - RNA, 21 nt, synthetic construct
  source 1..21: mol_type = other RNA
atgcctgggaacttcttgctc

SEQ ID NO: 127 - RNA, 21 nt, synthetic construct
  source 1..21: mol_type = other RNA
cattccagatgcctactactg

SEQ ID NO: 128 - RNA, 21 nt, synthetic construct
  source 1..21: mol_type = other RNA
taacccacagacacccatgac

SEQ ID NO: 129 - RNA, 21 nt, synthetic construct
  source 1..21: mol_type = other RNA
acacttccaaatttacagagc

SEQ ID NO: 130 - DNA, 19 bp, synthetic construct
  source 1..19: mol_type = other DNA
cgaagcgccctactccact

SEQ ID NO: 131 - RNA, 21 nt, synthetic construct
  misc_feature 1..21: note = artificial sequence
  source 1..21: mol_type = other RNA
gaccaccaggaactatatctt

SEQ ID NO: 132 - RNA, 21 nt, synthetic construct
  misc_feature 1..21: note = artificial sequence
  source 1..21: mol_type = other RNA
gttccagaaatacattggttt

SEQ ID NO: 133 - RNA, 21 nt, synthetic construct
  misc_feature 1..21: note = artificial sequence
  source 1..21: mol_type = other RNA
aaccgcaagggctttgtgaaa

SEQ ID NO: 134 - RNA, 21 nt, synthetic construct
  misc_feature 1..21: note = artificial sequence
  source 1..21: mol_type = other RNA
gagcaagaagttcccaggcat

SEQ ID NO: 135 - RNA, 21 nt, synthetic construct
  misc_feature 1..21: note = artificial sequence
  source 1..21: mol_type = other RNA
ctttggagagaatgaagtgta

SEQ ID NO: 136 - RNA, 21 nt, synthetic construct
  misc_feature 1..21: note = artificial sequence
  source 1..21: mol_type = other RNA
cttcgacaagcacaagaccaa

SEQ ID NO: 137 - RNA, 21 nt, synthetic construct
  misc_feature 1..21: note = artificial sequence
  source 1..21: mol_type = other RNA
gccgatgggtccagaagaagt

SEQ ID NO: 138 - RNA, 21 nt, synthetic construct
  misc_feature 1..21: note = artificial sequence
  source 1..21: mol_type = other RNA
cttcacttggctggtgtttga

SEQ ID NO: 139 - RNA, 21 nt, synthetic construct
  misc_feature 1..21: note = artificial sequence
  source 1..21: mol_type = other RNA
ctcctttggagagaatgaagt

SEQ ID NO: 140 - RNA, 21 nt, synthetic construct
  misc_feature 1..21: note = artificial sequence
  source 1..21: mol_type = other RNA
tgccatcctcatgtacatatt

SEQ ID NO: 141 - RNA, 21 nt, synthetic construct
  misc_feature 1..21: note = artificial sequence
  source 1..21: mol_type = other RNA
ccgcaagggctttgtgaaact

SEQ ID NO: 142 - RNA, 21 nt, synthetic construct
  misc_feature 1..21: note = artificial sequence
  source 1..21: mol_type = other RNA
agcaagaagttcccaggcata

SEQ ID NO: 143 - RNA, 21 nt, synthetic construct
  misc_feature 1..21: note = artificial sequence
  source 1..21: mol_type = other RNA
agtgtacaagcaggtgatctt

SEQ ID NO: 144 - RNA, 21 nt, synthetic construct
  misc_feature 1..21: note = artificial sequence
  source 1..21: mol_type = other RNA
tgctgaccaccaggaactata

SEQ ID NO: 145 - RNA, 21 nt, synthetic construct
  misc_feature 1..21: note = artificial sequence
  source 1..21: mol_type = other RNA
ccgatgggtccagaagaagtt

SEQ ID NO: 146 - RNA, 21 nt, synthetic construct
  misc_feature 1..21: note = artificial sequence
  source 1..21: mol_type = other RNA
tttggagagaatgaagtgtac

SEQ ID NO: 147 - RNA, 21 nt, synthetic construct
  misc_feature 1..21: note = artificial sequence
  source 1..21: mol_type = other RNA
tggcgctactttcgagactac

SEQ ID NO: 148 - RNA, 21 nt, synthetic construct
  misc_feature 1..21: note = artificial sequence
  source 1..21: mol_type = other RNA
aatgcctgtgttgagggagta

SEQ ID NO: 149 - RNA, 21 nt, synthetic construct
  misc_feature 1..21: note = artificial sequence
  source 1..21: mol_type = other RNA
agttccagaaatacattggtt

SEQ ID NO: 150 - RNA, 21 nt, synthetic construct
  misc_feature 1..21: note = artificial sequence
  source 1..21: mol_type = other RNA
cagaagtgagcaagaagttcc

SEQ ID NO: 151 - RNA, 21 nt, synthetic construct
  misc_feature 1..21: note = artificial sequence
  source 1..21: mol_type = other RNA
aagatatagttcctggtggtc

SEQ ID NO: 152 - RNA, 21 nt, synthetic construct
  misc_feature 1..21: note = artificial sequence
  source 1..21: mol_type = other RNA
aaaccaatgtatttctggaac

SEQ ID NO: 153 - RNA, 21 nt, synthetic construct
  misc_feature 1..21: note = artificial sequence
  source 1..21: mol_type = other RNA
tttcacaaagcccttgcggtt

SEQ ID NO: 154 - RNA, 21 nt, synthetic construct
  misc_feature 1..21: note = artificial sequence
  source 1..21: mol_type = other RNA
atgcctgggaacttcttgctc

SEQ ID NO: 155 - RNA, 21 nt, synthetic construct
  misc_feature 1..21: note = artificial sequence
  source 1..21: mol_type = other RNA
tacacttcattctctccaaag

SEQ ID NO: 156 - RNA, 21 nt, synthetic construct
  misc_feature 1..21: note = artificial sequence
  source 1..21: mol_type = other RNA
ttggtcttgtgcttgtcgaag

SEQ ID NO: 157 - RNA, 21 nt, synthetic construct
  misc_feature 1..21: note = artificial sequence
  source 1..21: mol_type = other RNA
acttcttctggacccatcggc

SEQ ID NO: 158 - RNA, 21 nt, synthetic construct
  misc_feature 1..21: note = artificial sequence
  source 1..21: mol_type = other RNA
tcaaacaccagccaagtgaag

SEQ ID NO: 159 - RNA, 21 nt, synthetic construct
  misc_feature 1..21: note = artificial sequence
  source 1..21: mol_type = other RNA
acttcattctctccaaaggag

SEQ ID NO: 160 - RNA, 21 nt, synthetic construct
  misc_feature 1..21: note = artificial sequence
  source 1..21: mol_type = other RNA
aatatgtacatgaggatggca

SEQ ID NO: 161 - RNA, 21 nt, synthetic construct
  misc_feature 1..21: note = artificial sequence
  source 1..21: mol_type = other RNA
agtttcacaaagcccttgcgg

SEQ ID NO: 162 - RNA, 21 nt, synthetic construct
  misc_feature 1..21: note = artificial sequence
  source 1..21: mol_type = other RNA
tatgcctgggaacttcttgct

SEQ ID NO: 163 - RNA, 21 nt, synthetic construct
  misc_feature 1..21: note = artificial sequence
  source 1..21: mol_type = other RNA
aagatcacctgcttgtacact

SEQ ID NO: 164 - RNA, 21 nt, synthetic construct
  misc_feature 1..21: note = artificial sequence
  source 1..21: mol_type = other RNA
tatagttcctggtggtcagca

SEQ ID NO: 165 - RNA, 21 nt, synthetic construct
  misc_feature 1..21: note = artificial sequence
  source 1..21: mol_type = other RNA
aacttcttctggacccatcgg

SEQ ID NO: 166 - RNA, 21 nt, synthetic construct
  misc_feature 1..21: note = artificial sequence
  source 1..21: mol_type = other RNA
gtacacttcattctctccaaa

SEQ ID NO: 167 - RNA, 21 nt, synthetic construct
  misc_feature 1..21: note = artificial sequence
  source 1..21: mol_type = other RNA
gtagtctcgaaagtagcgcca

SEQ ID NO: 168 - RNA, 21 nt, synthetic construct
  misc_feature 1..21: note = artificial sequence
  source 1..21: mol_type = other RNA
tactccctcaacacaggcatt

SEQ ID NO: 169 - RNA, 21 nt, synthetic construct
  misc_feature 1..21: note = artificial sequence
  source 1..21: mol_type = other RNA
aaccaatgtatttctggaact

SEQ ID NO: 170 - RNA, 21 nt, synthetic construct
  misc_feature 1..21: note = artificial sequence
  source 1..21: mol_type = other RNA
ggaacttcttgctcacttctg

SEQ ID NO: 171 - RNA, 21 nt, synthetic construct
  misc_feature 1..21: note = artificial sequence
  source 1..21: mol_type = other RNA
cctcataggcctggagtttat

SEQ ID NO: 172 - RNA, 21 nt, synthetic construct
  misc_feature 1..21: note = artificial sequence
  source 1..21: mol_type = other RNA
aggcctggagtttattcggaa

SEQ ID NO: 173 - RNA, 21 nt, synthetic construct
  misc_feature 1..21: note = artificial sequence
  source 1..21: mol_type = other RNA
ccctcataggcctggagttta

SEQ ID NO: 174 - RNA, 21 nt, synthetic construct
  misc_feature 1..21: note = artificial sequence
  source 1..21: mol_type = other RNA
aggtctggaatgcaaagtcaa

SEQ ID NO: 175 - RNA, 21 nt, synthetic construct
  misc_feature 1..21: note = artificial sequence
  source 1..21: mol_type = other RNA
ggcctggagtttattcggaaa

SEQ ID NO: 176 - RNA, 21 nt, synthetic construct
  misc_feature 1..21: note = artificial sequence
  source 1..21: mol_type = other RNA
caggtctggaatgcaaagtca

SEQ ID NO: 177 - RNA, 21 nt, synthetic construct
  misc_feature 1..21: note = artificial sequence
  source 1..21: mol_type = other RNA
cctcaccaagatcctgcatgt

SEQ ID NO: 178 - RNA, 21 nt, synthetic construct
  misc_feature 1..21: note = artificial sequence
  source 1..21: mol_type = other RNA
accctcataggcctggagttt

SEQ ID NO: 179 - RNA, 21 nt, synthetic construct
  misc_feature 1..21: note = artificial sequence
  source 1..21: mol_type = other RNA
caccagcatacagagtgacca

SEQ ID NO: 180 - RNA, 21 nt, synthetic construct
  misc_feature 1..21: note = artificial sequence
  source 1..21: mol_type = other RNA
atctcctagacaccagcatac

SEQ ID NO: 181 - RNA, 21 nt, synthetic construct
  misc_feature 1..21: note = artificial sequence
  source 1..21: mol_type = other RNA
tcctagacaccagcatacaga

SEQ ID NO: 182 - RNA, 21 nt, synthetic construct
  misc_feature 1..21: note = artificial sequence
  source 1..21: mol_type = other RNA
ctggagtttattcggaaaagc

SEQ ID NO: 183 - RNA, 21 nt, synthetic construct
  misc_feature 1..21: note = artificial sequence
  source 1..21: mol_type = other RNA
gcctggagtttattcggaaaa

SEQ ID NO: 184 - RNA, 21 nt, synthetic construct
  misc_feature 1..21: note = artificial sequence
  source 1..21: mol_type = other RNA
gaggcagagactgatccactt

SEQ ID NO: 185 - RNA, 21 nt, synthetic construct
  misc_feature 1..21: note = artificial sequence
  source 1..21: mol_type = other RNA
taggcctggagtttattcgga

SEQ ID NO: 186 - RNA, 21 nt, synthetic construct
  misc_feature 1..21: note = artificial sequence
  source 1..21: mol_type = other RNA
cacttctctgccaaagatgtc

SEQ ID NO: 187 - RNA, 21 nt, synthetic construct
  misc_feature 1..21: note = artificial sequence
  source 1..21: mol_type = other RNA
atgcaaagtcaaggagcatgg

SEQ ID NO: 188 - RNA, 21 nt, synthetic construct
  misc_feature 1..21: note = artificial sequence
  source 1..21: mol_type = other RNA
ggtcatggtcaccgacttcga

SEQ ID NO: 189 - RNA, 21 nt, synthetic construct
  misc_feature 1..21: note = artificial sequence
  source 1..21: mol_type = other RNA
ggcagctgttttgcaggactg

SEQ ID NO: 190 - RNA, 21 nt, synthetic construct
  misc_feature 1..21: note = artificial sequence
  source 1..21: mol_type = other RNA
gggcaggttggcagctgtttt

SEQ ID NO: 191 - RNA, 21 nt, synthetic construct
  misc_feature 1..21: note = artificial sequence
  source 1..21: mol_type = other RNA
ataaactccaggcctatgagg

SEQ ID NO: 192 - RNA, 21 nt, synthetic construct
  misc_feature 1..21: note = artificial sequence
  source 1..21: mol_type = other RNA
ttccgaataaactccaggcct

SEQ ID NO: 193 - RNA, 21 nt, synthetic construct
  misc_feature 1..21: note = artificial sequence
  source 1..21: mol_type = other RNA
taaactccaggcctatgaggg

SEQ ID NO: 194 - RNA, 21 nt, synthetic construct
  misc_feature 1..21: note = artificial sequence
  source 1..21: mol_type = other RNA
ttgactttgcattccagacct

SEQ ID NO: 195 - RNA, 21 nt, synthetic construct
  misc_feature 1..21: note = artificial sequence
  source 1..21: mol_type = other RNA
tttccgaataaactccaggcc

SEQ ID NO: 196 - RNA, 21 nt, synthetic construct
  misc_feature 1..21: note = artificial sequence
  source 1..21: mol_type = other RNA
tgactttgcattccagacctg

SEQ ID NO: 197 - RNA, 21 nt, synthetic construct
  misc_feature 1..21: note = artificial sequence
  source 1..21: mol_type = other RNA
acatgcaggatcttggtgagg

SEQ ID NO: 198 - RNA, 21 nt, synthetic construct
  misc_feature 1..21: note = artificial sequence
  source 1..21: mol_type = other RNA
aaactccaggcctatgagggt

SEQ ID NO: 199 - RNA, 21 nt, synthetic construct
  misc_feature 1..21: note = artificial sequence
  source 1..21: mol_type = other RNA
tggtcactctgtatgctggtg

SEQ ID NO: 200 - RNA, 21 nt, synthetic construct
  misc_feature 1..21: note = artificial sequence
  source 1..21: mol_type = other RNA
gtatgctggtgtctaggagat

SEQ ID NO: 201 - RNA, 21 nt, synthetic construct
  misc_feature 1..21: note = artificial sequence
  source 1..21: mol_type = other RNA
tctgtatgctggtgtctagga

SEQ ID NO: 202 - RNA, 21 nt, synthetic construct
  misc_feature 1..21: note = artificial sequence
  source 1..21: mol_type = other RNA
gcttttccgaataaactccag

SEQ ID NO: 203 - RNA, 21 nt, synthetic construct
  misc_feature 1..21: note = artificial sequence
  source 1..21: mol_type = other RNA
ttttccgaataaactccaggc

SEQ ID NO: 204 - RNA, 21 nt, synthetic construct
  misc_feature 1..21: note = artificial sequence
  source 1..21: mol_type = other RNA
aagtggatcagtctctgcctc

SEQ ID NO: 205 - RNA, 21 nt, synthetic construct
  misc_feature 1..21: note = artificial sequence
  source 1..21: mol_type = other RNA
tccgaataaactccaggccta

SEQ ID NO: 206 - RNA, 21 nt, synthetic construct
  misc_feature 1..21: note = artificial sequence
  source 1..21: mol_type = other RNA
gacatctttggcagagaagtg

SEQ ID NO: 207 - RNA, 21 nt, synthetic construct
  misc_feature 1..21: note = artificial sequence
  source 1..21: mol_type = other RNA
ccatgctccttgactttgcat

SEQ ID NO: 208 - RNA, 21 nt, synthetic construct
  misc_feature 1..21: note = artificial sequence
  source 1..21: mol_type = other RNA
tcgaagtcggtgaccatgacc

SEQ ID NO: 209 - RNA, 21 nt, synthetic construct
  misc_feature 1..21: note = artificial sequence
  source 1..21: mol_type = other RNA
cagtcctgcaaaacagctgcc

SEQ ID NO: 210 - RNA, 21 nt, synthetic construct
  misc_feature 1..21: note = artificial sequence
  source 1..21: mol_type = other RNA
aaaacagctgccaacctgccc

SEQ ID NO: 211 - RNA, 21 nt, synthetic construct
  misc_feature 1..21: note = artificial sequence
  source 1..21: mol_type = other RNA
ctggagcaccgttaaggacaa

SEQ ID NO: 212 - RNA, 21 nt, synthetic construct
  misc_feature 1..21: note = artificial sequence
  source 1..21: mol_type = other RNA
ccctgaaagactactggagca

SEQ ID NO: 213 - RNA, 21 nt, synthetic construct
  misc_feature 1..21: note = artificial sequence
  source 1..21: mol_type = other RNA
gagcaccgttaaggacaagtt

SEQ ID NO: 214 - RNA, 21 nt, synthetic construct
  misc_feature 1..21: note = artificial sequence
  source 1..21: mol_type = other RNA
actggagcaccgttaaggaca

SEQ ID NO: 215 - RNA, 21 nt, synthetic construct
  misc_feature 1..21: note = artificial sequence
  source 1..21: mol_type = other RNA
cctgaaagactactggagcac

SEQ ID NO: 216 - RNA, 21 nt, synthetic construct
  misc_feature 1..21: note = artificial sequence
  source 1..21: mol_type = other RNA
aagactactggagcaccgtta

SEQ ID NO: 217 - RNA, 21 nt, synthetic construct
  misc_feature 1..21: note = artificial sequence
  source 1..21: mol_type = other RNA
cagttccctgaaagactactg

SEQ ID NO: 218 - RNA, 21 nt, synthetic construct
  misc_feature 1..21: note = artificial sequence
  source 1..21: mol_type = other RNA
ggtgaccgatggcttcagttc

SEQ ID NO: 219 - RNA, 21 nt, synthetic construct
  misc_feature 1..21: note = artificial sequence
  source 1..21: mol_type = other RNA
gggtgaccgatggcttcagtt

SEQ ID NO: 220 - RNA, 21 nt, synthetic construct
  misc_feature 1..21: note = artificial sequence
  source 1..21: mol_type = other RNA
actactggagcaccgttaagg

SEQ ID NO: 221 - RNA, 21 nt, synthetic construct
  misc_feature 1..21: note = artificial sequence
  source 1..21: mol_type = other RNA
gactactggagcaccgttaag

SEQ ID NO: 222 - RNA, 21 nt, synthetic construct
  misc_feature 1..21: note = artificial sequence
  source 1..21: mol_type = other RNA
ttcagttccctgaaagactac

SEQ ID NO: 223 - RNA, 21 nt, synthetic construct
  misc_feature 1..21: note = artificial sequence
  source 1..21: mol_type = other RNA
gttccctgaaagactactgga

SEQ ID NO: 224 - RNA, 21 nt, synthetic construct
  misc_feature 1..21: note = artificial sequence
  source 1..21: mol_type = other RNA
tggagcaccgttaaggacaag

SEQ ID NO: 225 - RNA, 21 nt, synthetic construct
  misc_feature 1..21: note = artificial sequence
  source 1..21: mol_type = other RNA
cgccaccaagaccgccaagga

SEQ ID NO: 226 - RNA, 21 nt, synthetic construct
  misc_feature 1..21: note = artificial sequence
  source 1..21: mol_type = other RNA
gggctgggtgaccgatggctt

SEQ ID NO: 227 - RNA, 21 nt, synthetic construct
  misc_feature 1..21: note = artificial sequence
  source 1..21: mol_type = other RNA
gccaccaagaccgccaaggat

SEQ ID NO: 228 - RNA, 21 nt, synthetic construct
  misc_feature 1..21: note = artificial sequence
  source 1..21: mol_type = other RNA
agactactggagcaccgttaa

SEQ ID NO: 229 - RNA, 21 nt, synthetic construct
  misc_feature 1..21: note = artificial sequence
  source 1..21: mol_type = other RNA
ccaccaagaccgccaaggatg

SEQ ID NO: 230 - RNA, 21 nt, synthetic construct
  misc_feature 1..21: note = artificial sequence
  source 1..21: mol_type = other RNA
tccctgaaagactactggagc

SEQ ID NO: 231 - RNA, 21 nt, synthetic construct
  misc_feature 1..21: note = artificial sequence
  source 1..21: mol_type = other RNA
ttgtccttaacggtgctccag

SEQ ID NO: 232 - RNA, 21 nt, synthetic construct
  misc_feature 1..21: note = artificial sequence
  source 1..21: mol_type = other RNA
tgctccagtagtctttcaggg

SEQ ID NO: 233 - RNA, 21 nt, synthetic construct
  misc_feature 1..21: note = artificial sequence
  source 1..21: mol_type = other RNA
aacttgtccttaacggtgctc

SEQ ID NO: 234 - RNA, 21 nt, synthetic construct
  misc_feature 1..21: note = artificial sequence
  source 1..21: mol_type = other RNA
tgtccttaacggtgctccagt

SEQ ID NO: 235 - RNA, 21 nt, synthetic construct
  misc_feature 1..21: note = artificial sequence
  source 1..21: mol_type = other RNA
gtgctccagtagtctttcagg

SEQ ID NO: 236 - RNA, 21 nt, synthetic construct
  misc_feature 1..21: note = artificial sequence
  source 1..21: mol_type = other RNA
taacggtgctccagtagtctt

SEQ ID NO: 237 - RNA, 21 nt, synthetic construct
  misc_feature 1..21: note = artificial sequence
  source 1..21: mol_type = other RNA
cagtagtctttcagggaactg

SEQ ID NO: 238 - RNA, 21 nt, synthetic construct
  misc_feature 1..21: note = artificial sequence
  source 1..21: mol_type = other RNA
gaactgaagccatcggtcacc

SEQ ID NO: 239 - RNA, 21 nt, synthetic construct
  misc_feature 1..21: note = artificial sequence
  source 1..21: mol_type = other RNA
aactgaagccatcggtcaccc

SEQ ID NO: 240 - RNA, 21 nt, synthetic construct
  misc_feature 1..21: note = artificial sequence
  source 1..21: mol_type = other RNA
ccttaacggtgctccagtagt

SEQ ID NO: 241 - RNA, 21 nt, synthetic construct
  misc_feature 1..21: note = artificial sequence
  source 1..21: mol_type = other RNA
cttaacggtgctccagtagtc

SEQ ID NO: 242 - RNA, 21 nt, synthetic construct
  misc_feature 1..21: note = artificial sequence
  source 1..21: mol_type = other RNA
gtagtctttcagggaactgaa

SEQ ID NO: 243 - RNA, 21 nt, synthetic construct
  misc_feature 1..21: note = artificial sequence
  source 1..21: mol_type = other RNA
tccagtagtctttcagggaac

SEQ ID NO: 244 - RNA, 21 nt, synthetic construct
  misc_feature 1..21: note = artificial sequence
  source 1..21: mol_type = other RNA
cttgtccttaacggtgctcca

SEQ ID NO: 245 - RNA, 21 nt, synthetic construct
  misc_feature 1..21: note = artificial sequence
  source 1..21: mol_type = other RNA
tccttggcggtcttggtggcg

SEQ ID NO: 246 - RNA, 21 nt, synthetic construct
  misc_feature 1..21: note = artificial sequence
  source 1..21: mol_type = other RNA
aagccatcggtcacccagccc

SEQ ID NO: 247 - RNA, 21 nt, synthetic construct
  misc_feature 1..21: note = artificial sequence
  source 1..21: mol_type = other RNA
atccttggcggtcttggtggc

SEQ ID NO: 248 - RNA, 21 nt, synthetic construct
  misc_feature 1..21: note = artificial sequence
  source 1..21: mol_type = other RNA
ttaacggtgctccagtagtct

SEQ ID NO: 249 - RNA, 21 nt, synthetic construct
  misc_feature 1..21: note = artificial sequence
  source 1..21: mol_type = other RNA
catccttggcggtcttggtgg

SEQ ID NO: 250 - RNA, 21 nt, synthetic construct
  misc_feature 1..21: note = artificial sequence
  source 1..21: mol_type = other RNA
gctccagtagtctttcaggga

SEQ ID NO: 251 - DNA, 19 bp, synthetic construct
  source 1..19: mol_type = other DNA
  misc_feature 1..19: note = single stranded DNA
tcacctcatcccgcgaagc

SEQ ID NO: 252 - RNA, 23 nt, synthetic construct
  source 1..23: mol_type = other RNA
  modified_base 22..23: mod_base = OTHER; note = thymine
  misc_feature 1..23: note = artificial sequence
ttccgaataaactccaggccttt

SEQ ID NO: 253 - DNA, 40 bp, synthetic construct
  misc_feature 1..21: note = RNA
  source 1..40: mol_type = other DNA
  misc_feature 22..40: note = DNA
aggcctggagtttattcggaatcacctcatcccgcgaagc

SEQ ID NO: 254 - RNA, 23 nt, synthetic construct
  source 1..23: mol_type = other RNA
  modified_base 22..23: mod_base = OTHER; note = thymine
  misc_feature 1..23: note = artificial sequence
ttccgaataaactccaggccttt

SEQ ID NO: 255 - RNA, 21 nt, synthetic construct
  misc_feature 1..21: note = sense/antisense RNA
  source 1..21: mol_type = other RNA
cctcataggcctggagtttat

SEQ ID NO: 256 - RNA, 21 nt, synthetic construct
  misc_feature 1..21: note = sense/antisense RNA
  source 1..21: mol_type = other RNA
aggcctggagtttattcggaa

SEQ ID NO: 257 - RNA, 21 nt, synthetic construct
  misc_feature 1..21: note = sense/antisense RNA
  source 1..21: mol_type = other RNA
ccctcataggcctggagttta

SEQ ID NO: 258 - RNA, 21 nt, synthetic construct
  misc_feature 1..21: note = sense/antisense RNA
  source 1..21: mol_type = other RNA
accctcataggcctggagttt

SEQ ID NO: 259 - RNA, 21 nt, synthetic construct
  misc_feature 1..21: note = sense/antisense RNA
  source 1..21: mol_type = other RNA
taggcctggagtttattcgga

SEQ ID NO: 260 - RNA, 21 nt, synthetic construct
  misc_feature 1..21: note = sense/antisense RNA
  source 1..21: mol_type = other RNA
aggtctggaatgcaaagtcaa

SEQ ID NO: 261 - RNA, 21 nt, synthetic construct
  misc_feature 1..21: note = sense/antisense RNA
  source 1..21: mol_type = other RNA
ggcctggagtttattcggaaa

SEQ ID NO: 262 - RNA, 21 nt, synthetic construct
  misc_feature 1..21: note = sense/antisense RNA
  source 1..21: mol_type = other RNA
caggtctggaatgcaaagtca

SEQ ID NO: 263 - RNA, 21 nt, synthetic construct
  misc_feature 1..21: note = sense/antisense RNA
  source 1..21: mol_type = other RNA
cctcaccaagatcctgcatgt

SEQ ID NO: 264 - RNA, 21 nt, synthetic construct
  misc_feature 1..21: note = sense/antisense RNA
  source 1..21: mol_type = other RNA
caccagcatacagagtgacca

SEQ ID NO: 265 - RNA, 21 nt, synthetic construct
  misc_feature 1..21: note = sense/antisense RNA
  source 1..21: mol_type = other RNA
ataaactccaggcctatgagg

SEQ ID NO: 266 - RNA, 21 nt, synthetic construct
  misc_feature 1..21: note = sense/antisense RNA
  source 1..21: mol_type = other RNA
ttccgaataaactccaggcct

SEQ ID NO: 267 - RNA, 21 nt, synthetic construct
  misc_feature 1..21: note = sense/antisense RNA
  source 1..21: mol_type = other RNA
taaactccaggcctatgaggg

SEQ ID NO: 268 - RNA, 21 nt, synthetic construct
  misc_feature 1..21: note = sense/antisense RNA
  source 1..21: mol_type = other RNA
aaactccaggcctatgagggt

SEQ ID NO: 269 - RNA, 21 nt, synthetic construct
  misc_feature 1..21: note = sense/antisense RNA
  source 1..21: mol_type = other RNA
tccgaataaactccaggccta

SEQ ID NO: 270 - RNA, 21 nt, synthetic construct
  misc_feature 1..21: note = sense/antisense RNA
  source 1..21: mol_type = other RNA
ttgactttgcattccagacct

SEQ ID NO: 271 - RNA, 21 nt, synthetic construct
  misc_feature 1..21: note = sense/antisense RNA
  source 1..21: mol_type = other RNA
tttccgaataaactccaggcc

SEQ ID NO: 272 - RNA, 21 nt, synthetic construct
  misc_feature 1..21: note = sense/antisense RNA
  source 1..21: mol_type = other RNA
tgactttgcattccagacctg

SEQ ID NO: 273 - RNA, 21 nt, synthetic construct
  misc_feature 1..21: note = sense/antisense RNA
  source 1..21: mol_type = other RNA
acatgcaggatcttggtgagg

SEQ ID NO: 274 - RNA, 21 nt, synthetic construct
  misc_feature 1..21: note = sense/antisense RNA
  source 1..21: mol_type = other RNA
tggtcactctgtatgctggtg

SEQ ID NO: 275 - RNA, 21 nt, synthetic construct
  misc_feature 1..21: note = sense/antisense RNA
  source 1..21: mol_type = other RNA
agcaagcagacatttatcttt

SEQ ID NO: 276 - RNA, 21 nt, synthetic construct
  misc_feature 1..21: note = sense/antisense RNA
  source 1..21: mol_type = other RNA
aggtctggaatgcaaagtcaa

SEQ ID NO: 277 - RNA, 21 nt, synthetic construct
  misc_feature 1..21: note = sense/antisense RNA
  source 1..21: mol_type = other RNA
ggcctggagtttattcggaaa

SEQ ID NO: 278 - RNA, 21 nt, synthetic construct
  misc_feature 1..21: note = sense/antisense RNA
  source 1..21: mol_type = other RNA
caggtctggaatgcaaagtca

SEQ ID NO: 279 - RNA, 21 nt, synthetic construct
  misc_feature 1..21: note = sense/antisense RNA
  source 1..21: mol_type = other RNA
cccaagcaagcagacatttat

SEQ ID NO: 280 - RNA, 21 nt, synthetic construct
  misc_feature 1..21: note = sense/antisense RNA
  source 1..21: mol_type = other RNA
cctcaccaagatcctgcatgt

SEQ ID NO: 281 - RNA, 21 nt, synthetic construct
  misc_feature 1..21: note = sense/antisense RNA
  source 1..21: mol_type = other RNA
ttttctagacctgttttgctt

SEQ ID NO: 282 - RNA, 21 nt, synthetic construct
  misc_feature 1..21: note = sense/antisense RNA
  source 1..21: mol_type = other RNA
acccaagcaagcagacattta

SEQ ID NO: 283 - RNA, 21 nt, synthetic construct
  misc_feature 1..21: note = sense/antisense RNA
  source 1..21: mol_type = other RNA
caccagcatacagagtgacca

SEQ ID NO: 284 - RNA, 21 nt, synthetic construct
  misc_feature 1..21: note = sense/antisense RNA
  source 1..21: mol_type = other RNA
attctgggttttgtagcattt

SEQ ID NO: 285 - RNA, 21 nt, synthetic construct
  misc_feature 1..21: note = sense/antisense RNA
  source 1..21: mol_type = other RNA
atctcctagacaccagcatac

SEQ ID NO: 286 - RNA, 21 nt, synthetic construct
  misc_feature 1..21: note = sense/antisense RNA
  source 1..21: mol_type = other RNA
tcctagacaccagcatacaga

SEQ ID NO: 287 - RNA, 21 nt, synthetic construct
  misc_feature 1..21: note = sense/antisense RNA
  source 1..21: mol_type = other RNA
gacatttatcttttgggtctg

SEQ ID NO: 288 - RNA, 21 nt, synthetic construct
  misc_feature 1..21: note = sense/antisense RNA
  source 1..21: mol_type = other RNA
tattctgggttttgtagcatt

SEQ ID NO: 289 - RNA, 21 nt, synthetic construct
  misc_feature 1..21: note = sense/antisense RNA
  source 1..21: mol_type = other RNA
ctggagtttattcggaaaagc

SEQ ID NO: 290 - RNA, 21 nt, synthetic construct
  misc_feature 1..21: note = sense/antisense RNA
  source 1..21: mol_type = other RNA
gcctggagtttattcggaaaa

SEQ ID NO: 291 - RNA, 21 nt, synthetic construct
  misc_feature 1..21: note = sense/antisense RNA
  source 1..21: mol_type = other RNA
gaggcagagactgatccactt

SEQ ID NO: 292 - RNA, 21 nt, synthetic construct
  misc_feature 1..21: note = sense/antisense RNA
  source 1..21: mol_type = other RNA
aagcaagcagacatttatctt

SEQ ID NO: 293 - RNA, 21 nt, synthetic construct
  misc_feature 1..21: note = sense/antisense RNA
  source 1..21: mol_type = other RNA
tagacctgttttgcttttgta

SEQ ID NO: 294 - RNA, 21 nt, synthetic construct
  misc_feature 1..21: note = sense/antisense RNA
  source 1..21: mol_type = other RNA
tttgcttttgtaacttgaaga

SEQ ID NO: 295 - RNA, 21 nt, synthetic construct
  misc_feature 1..21: note = sense/antisense RNA
  source 1..21: mol_type = other RNA
cacttctctgccaaagatgtc

SEQ ID NO: 296 - RNA, 21 nt, synthetic construct
  misc_feature 1..21: note = sense/antisense RNA
  source 1..21: mol_type = other RNA
ttgcttttgtaacttgaagat

SEQ ID NO: 297 - RNA, 21 nt, synthetic construct
  misc_feature 1..21: note = sense/antisense RNA
  source 1..21: mol_type = other RNA
atgcaaagtcaaggagcatgg

SEQ ID NO: 298 - RNA, 21 nt, synthetic construct
  misc_feature 1..21: note = sense/antisense RNA
  source 1..21: mol_type = other RNA
cccacccaagcaagcagacat

SEQ ID NO: 299 - RNA, 21 nt, synthetic construct
  misc_feature 1..21: note = sense/antisense RNA
  source 1..21: mol_type = other RNA
gggtaacagtgaggctgggaa

SEQ ID NO: 300 - RNA, 21 nt, synthetic construct
  misc_feature 1..21: note = sense/antisense RNA
  source 1..21: mol_type = other RNA
ggtcatggtcaccgacttcga

SEQ ID NO: 301 - RNA, 21 nt, synthetic construct
  misc_feature 1..21: note = sense/antisense RNA
  source 1..21: mol_type = other RNA
ggcagctgttttgcaggactg

SEQ ID NO: 302 - RNA, 21 nt, synthetic construct
  misc_feature 1..21: note = sense/antisense RNA
  source 1..21: mol_type = other RNA
gggcaggttggcagctgtttt

SEQ ID NO: 303 - RNA, 21 nt, synthetic construct
  misc_feature 1..21: note = sense/antisense RNA
  source 1..21: mol_type = other RNA
ttgaagatatttattctgggt

SEQ ID NO: 304 - RNA, 21 nt, synthetic construct
  misc_feature 1..21: note = sense/antisense RNA
  source 1..21: mol_type = other RNA
tggcagctgttttgcaggact

SEQ ID NO: 305 - RNA, 21 nt, synthetic construct
  misc_feature 1..21: note = sense/antisense RNA
  source 1..21: mol_type = other RNA
ccggggatacctcaccaagat

SEQ ID NO: 306 - RNA, 21 nt, synthetic construct
  misc_feature 1..21: note = sense/antisense RNA
  source 1..21: mol_type = other RNA
actgatccacttctctgccaa

SEQ ID NO: 307 - RNA, 21 nt, synthetic construct
  misc_feature 1..21: note = sense/antisense RNA
  source 1..21: mol_type = other RNA
atccacttctctgccaaagat

SEQ ID NO: 308 - RNA, 21 nt, synthetic construct
  misc_feature 1..21: note = sense/antisense RNA
  source 1..21: mol_type = other RNA
acttctctgccaaagatgtca

SEQ ID NO: 309 - RNA, 21 nt, synthetic construct
  misc_feature 1..21: note = sense/antisense RNA
  source 1..21: mol_type = other RNA
gtctggaatgcaaagtcaagg

SEQ ID NO: 310 - RNA, 21 nt, synthetic construct
  misc_feature 1..21: note = sense/antisense RNA
  source 1..21: mol_type = other RNA
cttctctgccaaagatgtcat

SEQ ID NO: 311 - RNA, 21 nt, synthetic construct
  misc_feature 1..21: note = sense/antisense RNA
  source 1..21: mol_type = other RNA
gagttgaggcagagactgatc

SEQ ID NO: 312 - RNA, 21 nt, synthetic construct
  misc_feature 1..21: note = sense/antisense RNA
  source 1..21: mol_type = other RNA
gacctgttttgcttttgtaac

SEQ ID NO: 313 - RNA, 21 nt, synthetic construct
  misc_feature 1..21: note = sense/antisense RNA
  source 1..21: mol_type = other RNA
cggggatacctcaccaagatc

SEQ ID NO: 314 - RNA, 21 nt, synthetic construct
  misc_feature 1..21: note = sense/antisense RNA
  source 1..21: mol_type = other RNA
tttctagacctgttttgcttt

SEQ ID NO: 315 - RNA, 21 nt, synthetic construct
  misc_feature 1..21: note = sense/antisense RNA
  source 1..21: mol_type = other RNA
ggtctggaatgcaaagtcaag

SEQ ID NO: 316 - RNA, 21 nt, synthetic construct
  misc_feature 1..21: note = sense/antisense RNA
  source 1..21: mol_type = other RNA
tatctcctagacaccagcata

SEQ ID NO: 317 - RNA, 21 nt, synthetic construct
  misc_feature 1..21: note = sense/antisense RNA
  source 1..21: mol_type = other RNA
aggttggcagctgttttgcag

SEQ ID NO: 318 - RNA, 21 nt, synthetic construct
  misc_feature 1..21: note = sense/antisense RNA
  source 1..21: mol_type = other RNA
aacttttctagacctgttttg

SEQ ID NO: 319 - RNA, 21 nt, synthetic construct
  misc_feature 1..21: note = sense/antisense RNA
  source 1..21: mol_type = other RNA
cttttctagacctgttttgct

SEQ ID NO: 320 - RNA, 21 nt, synthetic construct
  misc_feature 1..21: note = sense/antisense RNA
  source 1..21: mol_type = other RNA
tccacttctctgccaaagatg

SEQ ID NO: 321 - RNA, 21 nt, synthetic construct
  misc_feature 1..21: note = sense/antisense RNA
  source 1..21: mol_type = other RNA
tggagtttattcggaaaagcc

SEQ ID NO: 322 - RNA, 21 nt, synthetic construct
  misc_feature 1..21: note = sense/antisense RNA
  source 1..21: mol_type = other RNA
ggcaggttggcagctgttttg

SEQ ID NO: 323 - RNA, 21 nt, synthetic construct
  misc_feature 1..21: note = sense/antisense RNA
  source 1..21: mol_type = other RNA
tggaggtgtatctcctagaca

SEQ ID NO: 324 - RNA, 21 nt, synthetic construct
  misc_feature 1..21: note = sense/antisense RNA
  source 1..21: mol_type = other RNA
gtcatcaatgaggcctggttc

SEQ ID NO: 325 - RNA, 21 nt, synthetic construct
  misc_feature 1..21: note = sense/antisense RNA
  source 1..21: mol_type = other RNA
ttctagacctgttttgctttt

SEQ ID NO: 326 - RNA, 21 nt, synthetic construct
  misc_feature 1..21: note = sense/antisense RNA
  source 1..21: mol_type = other RNA
ttctgggttttgtagcatttt

SEQ ID NO: 327 - RNA, 21 nt, synthetic construct
  misc_feature 1..21: note = sense/antisense RNA
  source 1..21: mol_type = other RNA
gagactgatccacttctctgc

SEQ ID NO: 328 - RNA, 21 nt, synthetic construct
  misc_feature 1..21: note = sense/antisense RNA
  source 1..21: mol_type = other RNA
agtcaaggagcatggaatccc

SEQ ID NO: 329 - RNA, 21 nt, synthetic construct
  misc_feature 1..21: note = sense/antisense RNA
  source 1..21: mol_type = other RNA
atcttttgggtctgtcctctc

SEQ ID NO: 330 - RNA, 21 nt, synthetic construct
  misc_feature 1..21: note = sense/antisense RNA
  source 1..21: mol_type = other RNA
cacccaagcaagcagacattt

SEQ ID NO: 331 - RNA, 21 nt, synthetic construct
  misc_feature 1..21: note = sense/antisense RNA
  source 1..21: mol_type = other RNA
aaagataaatgtctgcttgct

SEQ ID NO: 332 - RNA, 21 nt, synthetic construct
  misc_feature 1..21: note = sense/antisense RNA
  source 1..21: mol_type = other RNA
ttgactttgcattccagacct

SEQ ID NO: 333 - RNA, 21 nt, synthetic construct
  misc_feature 1..21: note = sense/antisense RNA
  source 1..21: mol_type = other RNA
tttccgaataaactccaggcc

SEQ ID NO: 334 - RNA, 21 nt, synthetic construct
  misc_feature 1..21: note = sense/antisense RNA
  source 1..21: mol_type = other RNA
tgactttgcattccagacctg

SEQ ID NO: 335 - RNA, 21 nt, synthetic construct
  misc_feature 1..21: note = sense/antisense RNA
  source 1..21: mol_type = other RNA
ataaatgtctgcttgcttggg

SEQ ID NO: 336 - RNA, 21 nt, synthetic construct
  misc_feature 1..21: note = sense/antisense RNA
  source 1..21: mol_type = other RNA
acatgcaggatcttggtgagg

SEQ ID NO: 337 - RNA, 21 nt, synthetic construct
  misc_feature 1..21: note = sense/antisense RNA
  source 1..21: mol_type = other RNA
aagcaaaacaggtctagaaaa

SEQ ID NO: 338 - RNA, 21 nt, synthetic construct
  misc_feature 1..21: note = sense/antisense RNA
  source 1..21: mol_type = other RNA
taaatgtctgcttgcttgggt

SEQ ID NO: 339 - RNA, 21 nt, synthetic construct
  misc_feature 1..21: note = sense/antisense RNA
  source 1..21: mol_type = other RNA
tggtcactctgtatgctggtg

SEQ ID NO: 340 - RNA, 21 nt, synthetic construct
  misc_feature 1..21: note = sense/antisense RNA
  source 1..21: mol_type = other RNA
aaatgctacaaaacccagaat

SEQ ID NO: 341 - RNA, 21 nt, synthetic construct
  misc_feature 1..21: note = sense/antisense RNA
  source 1..21: mol_type = other RNA
gtatgctggtgtctaggagat

SEQ ID NO: 342 - RNA, 21 nt, synthetic construct
  misc_feature 1..21: note = sense/antisense RNA
  source 1..21: mol_type = other RNA
tctgtatgctggtgtctagga

SEQ ID NO: 343 - RNA, 21 nt, synthetic construct
  misc_feature 1..21: note = sense/antisense RNA
  source 1..21: mol_type = other RNA
cagacccaaaagataaatgtc

SEQ ID NO: 344 - RNA, 21 nt, synthetic construct
  misc_feature 1..21: note = sense/antisense RNA
  source 1..21: mol_type = other RNA
aatgctacaaaacccagaata

SEQ ID NO: 345 - RNA, 21 nt, synthetic construct
  misc_feature 1..21: note = sense/antisense RNA
  source 1..21: mol_type = other RNA
gcttttccgaataaactccag

SEQ ID NO: 346 - RNA, 21 nt, synthetic construct
  misc_feature 1..21: note = sense/antisense RNA
  source 1..21: mol_type = other RNA
ttttccgaataaactccaggc

SEQ ID NO: 347 - RNA, 21 nt, synthetic construct
  misc_feature 1..21: note = sense/antisense RNA
  source 1..21: mol_type = other RNA
aagtggatcagtctctgcctc

SEQ ID NO: 348 - RNA, 21 nt, synthetic construct
  misc_feature 1..21: note = sense/antisense RNA
  source 1..21: mol_type = other RNA
aagataaatgtctgcttgctt

SEQ ID NO: 349 - RNA, 21 nt, synthetic construct
  misc_feature 1..21: note = sense/antisense RNA
  source 1..21: mol_type = other RNA
tacaaaagcaaaacaggtcta

SEQ ID NO: 350 - RNA, 21 nt, synthetic construct
  misc_feature 1..21: note = sense/antisense RNA
  source 1..21: mol_type = other RNA
tcttcaagttacaaaagcaaa

SEQ ID NO: 351 - RNA, 21 nt, synthetic construct
  misc_feature 1..21: note = sense/antisense RNA
  source 1..21: mol_type = other RNA
gacatctttggcagagaagtg

SEQ ID NO: 352 - RNA, 21 nt, synthetic construct
  misc_feature 1..21: note = sense/antisense RNA
  source 1..21: mol_type = other RNA
atcttcaagttacaaaagcaa

SEQ ID NO: 353 - RNA, 21 nt, synthetic construct
  misc_feature 1..21: note = sense/antisense RNA
  source 1..21: mol_type = other RNA
ccatgctccttgactttgcat

SEQ ID NO: 354 - RNA, 21 nt, synthetic construct
  misc_feature 1..21: note = sense/antisense RNA
  source 1..21: mol_type = other RNA
atgtctgcttgcttgggtggg

SEQ ID NO: 355 - RNA, 21 nt, synthetic construct
  misc_feature 1..21: note = sense/antisense RNA
  source 1..21: mol_type = other RNA
ttcccagcctcactgttaccc

SEQ ID NO: 356 - RNA, 21 nt, synthetic construct
  misc_feature 1..21: note = sense/antisense RNA
  source 1..21: mol_type = other RNA
tcgaagtcggtgaccatgacc

SEQ ID NO: 357 - RNA, 21 nt, synthetic construct
  misc_feature 1..21: note = sense/antisense RNA
  source 1..21: mol_type = other RNA
cagtcctgcaaaacagctgcc

SEQ ID NO: 358 - RNA, 21 nt, synthetic construct
  misc_feature 1..21: note = sense/antisense RNA
  source 1..21: mol_type = other RNA
aaaacagctgccaacctgccc

SEQ ID NO: 359 - RNA, 21 nt, synthetic construct
  misc_feature 1..21: note = sense/antisense RNA
  source 1..21: mol_type = other RNA
acccagaataaatatcttcaa

SEQ ID NO: 360 - RNA, 21 nt, synthetic construct
  misc_feature 1..21: note = sense/antisense RNA
  source 1..21: mol_type = other RNA
agtcctgcaaaacagctgcca

SEQ ID NO: 361 - RNA, 21 nt, synthetic construct
  misc_feature 1..21: note = sense/antisense RNA
  source 1..21: mol_type = other RNA
atcttggtgaggtatccccgg

SEQ ID NO: 362 - RNA, 21 nt, synthetic construct
  misc_feature 1..21: note = sense/antisense RNA
  source 1..21: mol_type = other RNA
ttggcagagaagtggatcagt

SEQ ID NO: 363 - RNA, 21 nt, synthetic construct
  misc_feature 1..21: note = sense/antisense RNA
  source 1..21: mol_type = other RNA
atctttggcagagaagtggat

SEQ ID NO: 364 - RNA, 21 nt, synthetic construct
  misc_feature 1..21: note = sense/antisense RNA
  source 1..21: mol_type = other RNA
tgacatctttggcagagaagt

SEQ ID NO: 365 - RNA, 21 nt, synthetic construct
  misc_feature 1..21: note = sense/antisense RNA
  source 1..21: mol_type = other RNA
ccttgactttgcattccagac

SEQ ID NO: 366 - RNA, 21 nt, synthetic construct
  misc_feature 1..21: note = sense/antisense RNA
  source 1..21: mol_type = other RNA
atgacatctttggcagagaag

SEQ ID NO: 367 - RNA, 21 nt, synthetic construct
  misc_feature 1..21: note = sense/antisense RNA
  source 1..21: mol_type = other RNA
gatcagtctctgcctcaactc

SEQ ID NO: 368 - RNA, 21 nt, synthetic construct
  misc_feature 1..21: note = sense/antisense RNA
  source 1..21: mol_type = other RNA
gttacaaaagcaaaacaggtc

SEQ ID NO: 369 - RNA, 21 nt, synthetic construct
  misc_feature 1..21: note = sense/antisense RNA
  source 1..21: mol_type = other RNA
gatcttggtgaggtatccccg

SEQ ID NO: 370 - RNA, 21 nt, synthetic construct
  misc_feature 1..21: note = sense/antisense RNA
  source 1..21: mol_type = other RNA
aaagcaaaacaggtctagaaa

SEQ ID NO: 371 - RNA, 21 nt, synthetic construct
  misc_feature 1..21: note = sense/antisense RNA
  source 1..21: mol_type = other RNA
cttgactttgcattccagacc

SEQ ID NO: 372 - RNA, 21 nt, synthetic construct
  misc_feature 1..21: note = sense/antisense RNA
  source 1..21: mol_type = other RNA
tatgctggtgtctaggagata

SEQ ID NO: 373 - RNA, 21 nt, synthetic construct
  misc_feature 1..21: note = sense/antisense RNA
  source 1..21: mol_type = other RNA
ctgcaaaacagctgccaacct

SEQ ID NO: 374 - RNA, 21 nt, synthetic construct
  misc_feature 1..21: note = sense/antisense RNA
  source 1..21: mol_type = other RNA
caaaacaggtctagaaaagtt

SEQ ID NO: 375 - RNA, 21 nt, synthetic construct
  misc_feature 1..21: note = sense/antisense RNA
  source 1..21: mol_type = other RNA
agcaaaacaggtctagaaaag

SEQ ID NO: 376 - RNA, 21 nt, synthetic construct
  misc_feature 1..21: note = sense/antisense RNA
  source 1..21: mol_type = other RNA
catctttggcagagaagtgga

SEQ ID NO: 377 - RNA, 21 nt, synthetic construct
  misc_feature 1..21: note = sense/antisense RNA
  source 1..21: mol_type = other RNA
ggcttttccgaataaactcca

SEQ ID NO: 378 - RNA, 21 nt, synthetic construct
  misc_feature 1..21: note = sense/antisense RNA
  source 1..21: mol_type = other RNA
caaaacagctgccaacctgcc

SEQ ID NO: 379 - RNA, 21 nt, synthetic construct
  misc_feature 1..21: note = sense/antisense RNA
  source 1..21: mol_type = other RNA
tgtctaggagatacacctcca

SEQ ID NO: 380 - RNA, 21 nt, synthetic construct
  misc_feature 1..21: note = sense/antisense RNA
  source 1..21: mol_type = other RNA
gaaccaggcctcattgatgac

SEQ ID NO: 381 - RNA, 21 nt, synthetic construct
  misc_feature 1..21: note = sense/antisense RNA
  source 1..21: mol_type = other RNA
aaaagcaaaacaggtctagaa

SEQ ID NO: 382 - RNA, 21 nt, synthetic construct
  misc_feature 1..21: note = sense/antisense RNA
  source 1..21: mol_type = other RNA
aaaatgctacaaaacccagaa

SEQ ID NO: 383 - RNA, 21 nt, synthetic construct
  misc_feature 1..21: note = sense/antisense RNA
  source 1..21: mol_type = other RNA
gcagagaagtggatcagtctc

SEQ ID NO: 384 - RNA, 21 nt, synthetic construct
  misc_feature 1..21: note = sense/antisense RNA
  source 1..21: mol_type = other RNA
gggattccatgctccttgact

SEQ ID NO: 385 - RNA, 21 nt, synthetic construct
  misc_feature 1..21: note = sense/antisense RNA
  source 1..21: mol_type = other RNA
gagaggacagacccaaaagat

SEQ ID NO: 386 - RNA, 21 nt, synthetic construct
  misc_feature 1..21: note = sense/antisense RNA
  source 1..21: mol_type = other RNA
aaatgtctgcttgcttgggtg

SEQ ID NO: 387 - DNA, 19 bp, synthetic construct
  misc_feature 1..19: note = crook sequence
  source 1..19: mol_type = other DNA
tcacctcatcccgcgaagc

SEQ ID NO: 388 - DNA, 3637 bp, synthetic construct
  misc_feature 1..3637: note = PCSK9 cDNA sequence
  source 1..3637: mol_type = other DNA
agcgacgtcgaggcgctcatggttgcaggcgggcgccgccgttcagttcagggtctgagcctggaggagtgagccaggcagtgagactggctcgggcgggccgggacgcgtcgttgcagcagcggctcccagctcccagccaggattccgcgcgccccttcacgcgccctgctcctgaacttcagctcctgcacagtcctccccaccgcaaggctcaaggcgccgccggcgtggaccgcgcacggcctctaggtctcctcgccaggacagcaacctctcccctggccctcatgggcaccgtcagctccaggcggtcctggtggccgctgccactgctgctgctgctgctgctgctcctgggtcccgcgggcgcccgtgcgcaggaggacgaggacggcgactacgaggagctggtgctagccttgcgttccgaggaggacggcctggccgaagcacccgagcacggaaccacagccaccttccaccgctgcgccaaggatccgtggaggttgcctggcacctacgtggtggtgctgaaggaggagacccacctctcgcagtcagagcgcactgcccgccgcctgcaggcccaggctgcccgccggggatacctcaccaagatcctgcatgtcttccatggccttcttcctggcttcctggtgaagatgagtggcgacctgctggagctggccttgaagttgccccatgtcgactacatcgaggaggactcctctgtctttgcccagagcatcccgtggaacctggagcggattacccctccacggtaccgggcggatgaataccagccccccgacggaggcagcctggtggaggtgtatctcctagacaccagcatacagagtgaccaccgggaaatcgagggcagggtcatggtcaccgacttcgagaatgtgcccgaggaggacgggacccgcttccacagacaggccagcaagtgtgacagtcatggcacccacctggcaggggtggtcagcggccgggatgccggcgtggccaagggtgccagcatgcgcagcctgcgcgtgctcaactgccaagggaagggcacggttagcggcaccctcataggcctggagtttattcggaaaagccagctggtccagcctgtggggccactggtggtgctgctgcccctggcgggtgggtacagccgcgtcctcaacgccgcctgccagcgcctggcgagggctggggtcgtgctggtcaccgctgccggcaacttccgggacgatgcctgcctctactccccagcctcagctcccgaggtcatcacagttggggccaccaatgcccaagaccagccggtgaccctggggactttggggaccaactttggccgctgtgtggacctctttgccccaggggaggacatcattggtgcctccagcgactgcagcacctgctttgtgtcacagagtgggacatcacaggctgctgcccacgtggctggcattgcagccatgatgctgtctgccgagccggagctcaccctggccgagttgaggcagagactgatccacttctctgccaaagatgtcatcaatgaggcctggttccctgaggaccagcgggtactgacccccaacctggtggccgccctgccccccagcacccatggggcaggttggcagctgttttgcaggactgtatggtcagcacactcggggcctacacggatggccacagccgtcgcccgctgcgccccagatgaggagctgctgagctgctccagtttctccaggagtgggaagcggcggggcgagcgcatggaggcccaagggggcaagctggtctgccgggcccacaacgcttttgggggtgagggtgtctacgccattgccaggtgctgcctgctaccccaggccaactgcagcgtccacacagctccaccagctgaggccagcatggggacccgtgtccactgccaccaacagggccacgtcctcacaggctgcagctcccactgggaggtggaggaccttggcacccacaagccgcctgtgctgaggccacgaggtcagcccaaccagtgcgtgggccacagggaggccagcatccacgcttcctgctgccatgccccaggtctggaatgcaaagtcaaggagcatggaatcccggcccctcaggagcaggtgaccgtggcctgcgaggagggctggaccctgactggctgcagtgccctccctgggacctcccacgtcctgggggcctacgccgtagacaacacgtgtgtagtcaggagccgggacgtcagcactacaggcagcaccagcgaaggggccgtgacagccgttgccatctgctgccggagccggcacctggcgcaggcctcccaggagctccagtgacagccccatcccaggatgggtgtctggggagggtcaagggctggggctgagctttaaaatggttccgacttgtccctctctcagccctccatggcctggcacgaggggatggggatgcttccgcctttccggggctgctggcctggcccttgagtggggcagcctccttgcctggaactcactcactctgggtgcctcctccccaggtggaggtgccaggaagctccctccctcactgtggggcatttcaccattcaaacaggtcgagctgtgctcgggtgctgccagctgctcccaatgtgccgatgtccgtgggcagaatgacttttattgagctcttgttccgtgccaggcattcaatcctcaggtctccaccaaggaggcaggattcttcccatggataggggagggggcggtaggggctgcagggacaaacatcgttggggggtgagtgtgaaaggtgctgatggccctcatctccagctaactgtggagaagcccctgggggctccctgattaatggaggcttagctttctggatggcatctagccagaggctggagacaggtgcgcccctggtggtcacaggctgtgccttggtttcctgagccacctttactctgctctatgccaggctgtgctagcaacacccaaaggtggcctgcggggagccatcacctaggactgactcggcagtgtgcagtggtgcatgcactgtctcagccaacccgctccactacccggcagggtacacattcgcacccctacttcacagaggaagaaacctggaaccagagggggcgtgcctgccaagctcacacagcaggaactgagccagaaacgcagattgggctggctctgaagccaagcctcttcttacttcacccggctgggctcctcatttttacgggtaacagtgaggctgggaaggggaacacagaccaggaagctcggtgagtgatggcagaacgatgcctgcaggcatggaactttttccgttatcacccaggcctgattcactggcctggcggagatgcttctaaggcatggtcgggggagagggccaacaactgtccctccttgagcaccagccccacccaagcaagcagacatttatcttttgggtctgtcctctctgttgcctttttacagccaacttttctagacctgttttgcttttgtaacttgaagatatttattctgggttttgtagcatttttattaatatggtgactttttaaaataaaaacaaacaaacgttgtcctaa

SEQ ID NO: 389 - RNA, 21 nt, synthetic construct
  modified_base 11: mod_base = OTHER; note = thymine
  misc_feature 1..21: note = sense unmodified inclisiran
  source 1..21: mol_type = other RNA
ctagacctgttttgcttttgt

SEQ ID NO: 390 - RNA, 23 nt, synthetic construct
  misc_feature 1..23: note = antisense unmodified inclisiran
  source 1..23: mol_type = other RNA
acaaaagcaaaacaggtctagaa

SEQ ID NO: 391 - RNA, 21 nt, synthetic construct
  misc_feature 1..21: note = apoB-1 sense
  source 1..21: mol_type = other RNA
gtcatcacactgaataccaat

SEQ ID NO: 392 - RNA, 23 nt, synthetic construct
  misc_feature 1..23: note = apoB-1 antisense
  source 1..23: mol_type = other RNA
attggtattcagtgtgatgacac

SEQ ID NO: 393 - RNA, 21 nt, synthetic construct
  misc_feature 1..21: note = beta-Gal sense
  source 1..21: mol_type = other RNA
ttgatgtgtttagtcgctatt

SEQ ID NO: 394 - RNA, 21 nt, synthetic construct
  misc_feature 1..21: note = beta-Gal antisense
  source 1..21: mol_type = other RNA
tagcgactaaacacatcaatt

SEQ ID NO: 395 - RNA, 19 nt, synthetic construct
  misc_feature 1..19: note = beta-gal 728 sense
  source 1..19: mol_type = other RNA
ctacacaaatcagcgattt

SEQ ID NO: 396 - RNA, 19 nt, synthetic construct
  misc_feature 1..19: note = beta-gal 728 antisense
  source 1..19: mol_type = other RNA
aaatcgctgatttgtgtag

SEQ ID NO: 397 - RNA, 21 nt, synthetic construct
  modified_base 20..21: mod_base = OTHER; note = thymine
  misc_feature 1..21: note = Luc-siRNA sense; deoxythymidine (20)..(21); DNA/RNA type sequence
  source 1..21: mol_type = other RNA
taaggctatgaagagatactt

SEQ ID NO: 398 - RNA, 21 nt, synthetic construct
  misc_feature 1..21: note = Luc-siRNA antisense
  source 1..21: mol_type = other RNA
aagtatctcttcatagcctta

SEQ ID NO: 399 - RNA, 23 nt, synthetic construct
  misc_feature 1..23: note = Antisense unmodified inclisiran
  source 1..23: mol_type = other RNA
acaaaagcaaaacaggtctagaa

SEQ ID NO: 400 - DNA, 19 bp, synthetic construct
  misc_feature 1..19: note = crook
  source 1..19: mol_type = other DNA
gcgaagcccctactccact

SEQ ID NO: 401 - DNA, 40 bp, synthetic construct
  source 1..40: mol_type = other DNA
  misc_feature 1..21: note = RNA
  misc_feature 22..40: note = DNA
acgggacagtattctcagtnatcacctcatcccgcgaagc

SEQ ID NO: 402 - DNA, 40 bp, synthetic construct
  source 1..40: mol_type = other DNA
  misc_feature 1..21: note = RNA
  misc_feature 22..40: note = DNA
cccaataaagctggacaagaatcacctcatcccgcgaagc

SEQ ID NO: 403 - DNA, 40 bp, synthetic construct
  source 1..40: mol_type = other DNA
  misc_feature 1..21: note = RNA
  misc_feature 22..40: note = DNA
ctgtaggttgcttaaaagggatcacctcatcccgcgaagc

SEQ ID NO: 404 - DNA, 40 bp, synthetic construct
  source 1..40: mol_type = other DNA
  misc_feature 1..21: note = RNA
  misc_feature 22..40: note = DNA
ctggagcaccgttaaggacaatcacctcatcccgcgaagc

SEQ ID NO: 405 - DNA, 40 bp, synthetic construct
  source 1..40: mol_type = other DNA
  misc_feature 1..21: note = RNA
  misc_feature 22..40: note = DNA
tcccaataaagctggacaagatcacctcatcccgcgaagc

SEQ ID NO: 406 - DNA, 40 bp, synthetic construct
  source 1..40: mol_type = other DNA
  misc_feature 1..21: note = RNA
  misc_feature 22..40: note = DNA
gcccctgtaggttgcttaaaatcacctcatcccgcgaagc

SEQ ID NO: 407 - DNA, 40 bp, synthetic construct
  source 1..40: mol_type = other DNA
  misc_feature 1..21: note = RNA
  misc_feature 22..40: note = DNA
ccctgaaagactactggagcatcacctcatcccgcgaagc

SEQ ID NO: 408 - DNA, 40 bp, synthetic construct
  source 1..40: mol_type = other DNA
  misc_feature 1..21: note = RNA
  misc_feature 22..40: note = DNA
tgcttaaaagggacagtattctcacctcatcccgcgaagc

SEQ ID NO: 409 - DNA, 40 bp, synthetic construct
  source 1..40: mol_type = other DNA
  misc_feature 1..21: note = RNA
  misc_feature 22..40: note = DNA
gacctcaataccccaagtccatcacctcatcccgcgaagc

SEQ ID NO: 410 - DNA, 40 bp, synthetic construct
  source 1..40: mol_type = other DNA
  misc_feature 1..21: note = RNA
  misc_feature 22..40: note = DNA
gagcaccgttaaggacaagtttcacctcatcccgcgaagc

SEQ ID NO: 411 - DNA, 40 bp, synthetic construct
  source 1..40: mol_type = other DNA
  misc_feature 1..21: note = RNA
  misc_feature 22..40: note = DNA
ctctgtaaatttggaagtgtctcacctcatcccgcgaagc

SEQ ID NO: 412 - DNA, 40 bp, synthetic construct
  source 1..40: mol_type = other DNA
  misc_feature 1..21: note = RNA
  misc_feature 22..40: note = DNA
caccatgagctaggtggagtatcacctcatcccgcgaagc

SEQ ID NO: 413 - DNA, 40 bp, synthetic construct
  source 1..40: mol_type = other DNA
  misc_feature 1..21: note = RNA
  misc_feature 22..40: note = DNA
ttcctgaagtgacaaaggaaatcacctcatcccgcgaagc

SEQ ID NO: 414 - DNA, 40 bp, synthetic construct
  source 1..40: mol_type = other DNA
  misc_feature 1..21: note = RNA
  misc_feature 22..40: note = DNA
gaccaccaggaactatatctttcacctcatcccgcgaagc

SEQ ID NO: 415 - DNA, 40 bp, synthetic construct
  source 1..40: mol_type = other DNA
  misc_feature 1..21: note = RNA
  misc_feature 22..40: note = DNA
gttccagaaatacattggttttcacctcatcccgcgaagc

SEQ ID NO: 416 - DNA, 40 bp, synthetic construct
  source 1..40: mol_type = other DNA
  misc_feature 1..21: note = RNA
  misc_feature 22..40: note = DNA
aaccgcaagggctttgtgaaatcacctcatcccgcgaagc

SEQ ID NO: 417 - DNA, 40 bp, synthetic construct
  source 1..40: mol_type = other DNA
  misc_feature 1..21: note = RNA
  misc_feature 22..40: note = DNA
gagcaagaagttcccaggcattcacctcatcccgcgaagc

SEQ ID NO: 418 - DNA, 40 bp, synthetic construct
  source 1..40: mol_type = other DNA
  misc_feature 1..21: note = RNA
  misc_feature 22..40: note = DNA
cagtagtaggcatctggaatgtcacctcatcccgcgaagc

SEQ ID NO: 419 - DNA, 40 bp, synthetic construct
  source 1..40: mol_type = other DNA
  misc_feature 1..21: note = RNA
  misc_feature 22..40: note = DNA
gtcatgggtgtctgtgggttatcacctcatcccgcgaagc

SEQ ID NO: 420 - DNA, 40 bp, synthetic construct
  source 1..40: mol_type = other DNA
  misc_feature 1..21: note = RNA
  misc_feature 22..40: note = DNA
gctctgtaaatttggaagtgttcacctcatcccgcgaagc

SEQ ID NO: 421 - RNA, 23 nt, synthetic construct
  modified_base 22..23: mod_base = OTHER; note = thymine
  source 1..23: mol_type = other RNA
  ncRNA 1..23: ncRNA_class = siRNA
caggtctggaatgcaaagtcatt

SEQ ID NO: 422 - RNA, 23 nt, synthetic construct
  modified_base 22..23: mod_base = OTHER; note = thymine
  source 1..23: mol_type = other RNA
  ncRNA 1..23: ncRNA_class = siRNA
tgactttgcattccagacctgtt

SEQ ID NO: 423 - DNA, 40 bp, synthetic construct
  source 1..40: mol_type = other DNA
  misc_feature 1..21: note = RNA
  misc_feature 22..40: note = DNA
  ncRNA 1..21: ncRNA_class = siRNA
caggtctggaatgcaaagtcatcacctcatcccgcgaagc

SEQ ID NO: 424 - DNA, 40 bp, synthetic construct
  source 1..40: mol_type = other DNA
  misc_feature 1..21: note = RNA
  misc_feature 22..40: note = DNA
  ncRNA 1..21: ncRNA_class = siRNA
tgactttgcattccagacctgtcacctcatcccgcgaagc

SEQ ID NO: 425 - DNA, 40 bp, synthetic construct
  source 1..40: mol_type = other DNA
  misc_feature 1..19: note = DNA
  misc_feature 20..40: note = RNA
  ncRNA 20..40: ncRNA_class = siRNA
cgaagcgccctactccactcaggtctggaatgcaaagtca

SEQ ID NO: 426 - DNA, 42 bp, synthetic construct
  source 1..42: mol_type = other DNA
  misc_feature order(1..19,41..42): note = DNA
  misc_feature 20..40: note = RNA
  ncRNA 20..40: ncRNA_class = siRNA
cgaagcgccctactccactcaggtctggaatgcaaagtcatt

SEQ ID NO: 427 - DNA, 40 bp, synthetic construct
  source 1..40: mol_type = other DNA
  misc_feature 1..19: note = DNA
  misc_feature 20..40: note = RNA
  ncRNA 20..40: ncRNA_class = siRNA
cgaagcgccctactccacttgactttgcattccagacctg

SEQ ID NO: 428 - DNA, 40 bp, synthetic construct
  source 1..40: mol_type = other DNA
  misc_feature 1..19: note = DNA
  misc_feature 20..40: note = RNA
  ncRNA 20..40: ncRNA_class = siRNA
  modified_base 20..22: mod_base = OTHER; note = Phosphorothioate internucleotide linkage
cgaagcgccctactccactaggcctggagtttattcggaa

SEQ ID NO: 429 - RNA, 21 nt, synthetic construct
  source 1..21: mol_type = other RNA
  ncRNA 1..21: ncRNA_class = siRNA
ttccgaataaactccaggcct

SEQ ID NO: 430 - RNA, 28 nt, synthetic construct
  source 1..28: mol_type = other RNA
  ncRNA 1..28: ncRNA_class = siRNA
  modified_base 11: mod_base = OTHER; note = thymine
ctagacctgttttgcttttgtgcgaagc

SEQ ID NO: 431 - DNA, 33 bp, synthetic construct
  source 1..33: mol_type = other DNA
  misc_feature 1..12: note = DNA
  misc_feature 13..33: note = RNA
  modified_base 23: mod_base = OTHER; note = thymine
  ncRNA 13..33: ncRNA_class = siRNA
ccctactccactctagacctgttttgcttttgt

SEQ ID NO: 432 - DNA, 28 bp, synthetic construct
  source 1..28: mol_type = other DNA
  misc_feature 1..7: note = DNA
  misc_feature 8..28: note = RNA
  ncRNA 8..28: ncRNA_class = siRNA
  modified_base 18: mod_base = OTHER; note = thymine
cgaagcgctagacctgttttgcttttgt

SEQ ID NO: 433 - DNA, 40 bp, synthetic construct; identical to SEQ ID NO: 423
  source 1..40: mol_type = other DNA
  misc_feature 1..21: note = RNA
  ncRNA 1..21: ncRNA_class = siRNA
  misc_feature 22..40: note = DNA

SEQ ID NO: 434 - DNA, 28 bp, synthetic construct
  source 1..28: mol_type = other DNA
  misc_feature 1..21: note = RNA
  misc_feature 22..28: note = DNA
  ncRNA 1..21: ncRNA_class = siRNA
caggtctggaatgcaaagtcagcgaagc

SEQ ID NO: 435 - DNA, 40 bp, synthetic construct; identical to SEQ ID NO: 425
  source 1..40: mol_type = other DNA
  misc_feature 1..19: note = DNA
  misc_feature 20..40: note = RNA
  ncRNA 20..40: ncRNA_class = siRNA

SEQ ID NO: 436 - DNA, 33 bp, synthetic construct
  source 1..33: mol_type = other DNA
  misc_feature 1..12: note = DNA
  misc_feature 13..33: note = RNA
  ncRNA 13..33: ncRNA_class = siRNA
ccctactccactcaggtctggaatgcaaagtca

SEQ ID NO: 437 - DNA, 40 bp, synthetic construct; identical to SEQ ID NO: 428
  source 1..40: mol_type = other DNA
  misc_feature 1..19: note = DNA
  misc_feature 20..40: note = RNA
  modified_base 20..22: mod_base = OTHER; note = Phosphorothioate internucleotide linkage
  ncRNA 20..40: ncRNA_class = siRNA

SEQ ID NO: 438 - DNA, 40 bp, synthetic construct; identical to SEQ ID NO: 253
  source 1..40: mol_type = other DNA
  misc_feature 1..21: note = RNA
  misc_feature 22..40: note = DNA
  ncRNA 22..40: ncRNA_class = siRNA
  modified_base 1..3: mod_base = OTHER; note = Phosphorothioate internucleotide linkage

SEQ ID NO: 439 - DNA, 38 bp, synthetic construct
  source 1..38: mol_type = other DNA
  misc_feature 1..19: note = DNA
  misc_feature 20..38: note = RNA
  ncRNA 20..38: ncRNA_class = siRNA
  modified_base 36..38: mod_base = OTHER; note = Phosphorothioate internucleotide linkage
cgaagcgccctactccacttagacttcctgaataacta

SEQ ID NO: 440 - RNA, 21 nt, synthetic construct
  source 1..21: mol_type = other RNA
  ncRNA 1..21: ncRNA_class = siRNA
  modified_base order(1..3,19..21): mod_base = OTHER; note = Phosphorothioate internucleotide linkage
tagttattcaggaagtctatt

SEQ ID NO: 441 - DNA, 38 bp, synthetic construct
  source 1..38: mol_type = other DNA
  modified_base 1..3: mod_base = OTHER; note = Phosphorothioate internucleotide linkage
  misc_feature 1..19: note = RNA
  misc_feature 20..38: note = DNA
  ncRNA 1..19: ncRNA_class = siRNA
tagacttcctgaataactatcacctcatcccgcgaagc

SEQ ID NO: 442 - RNA, 19 nt, synthetic construct
  source 1..19: mol_type = other RNA
  modified_base order(1..3,17..19): mod_base = OTHER; note = Phosphorothioate internucleotide linkage
  ncRNA 1..19: ncRNA_class = siRNA
tagacttcctgaataacta

SEQ ID NO: 443 - DNA, 40 bp, synthetic construct
  source 1..40: mol_type = other DNA
  misc_feature 1..21: note = RNA
  misc_feature 22..40: note = DNA
  ncRNA 1..21: ncRNA_class = siRNA
  modified_base order(1..3,19..21): mod_base = OTHER; note = Phosphorothioate internucleotide linkage
tagttattcaggaagtctatttcacctcatcccgcgaagc

SEQ ID NO: 444 - DNA, 38 bp, synthetic construct
  source 1..38: mol_type = other DNA
  modified_base 36..38: mod_base = OTHER; note = Phosphorothioate internucleotide linkage
  misc_feature 1..19: note = DNA
  misc_feature 20..38: note = RNA
  ncRNA 20..38: ncRNA_class = siRNA
cgaagcgccctactccacttcatcacactgaataccaa

SEQ ID NO: 445 - RNA, 21 nt, synthetic construct
  source 1..21: mol_type = other RNA
  ncRNA 1..21: ncRNA_class = siRNA
  modified_base order(1..3,19..21): mod_base = OTHER; note = Phosphorothioate internucleotide linkage
ttggtattcagtgtgatgatt

SEQ ID NO: 446 - DNA, 38 bp, synthetic construct
  source 1..38: mol_type = other DNA
  modified_base 1..3: mod_base = OTHER; note = Phosphorothioate internucleotide linkage
  misc_feature 1..19: note = RNA
  misc_feature 20..38: note = DNA
  ncRNA 1..19: ncRNA_class = siRNA
tcatcacactgaataccaatcacctcatcccgcgaagc

SEQ ID NO: 447 - RNA, 19 nt, synthetic construct
  source 1..19: mol_type = other RNA
  ncRNA 1..19: ncRNA_class = siRNA
  modified_base order(1..3,17..19): mod_base = OTHER; note = Phosphorothioate internucleotide linkage
tcatcacactgaataccaa

SEQ ID NO: 448 - DNA, 40 bp, synthetic construct
  source 1..40: mol_type = other DNA
  misc_feature 1..21: note = RNA
  misc_feature 22..40: note = DNA
  modified_base order(1..3,19..21): mod_base = OTHER; note = Phosphorothioate internucleotide linkage
  ncRNA 1..21: ncRNA_class = siRNA
ttggtattcagtgtgatgatttcacctcatcccgcgaagc

SEQ ID NO: 449 - DNA, 40 bp, synthetic construct
  source 1..40: mol_type = other DNA
  modified_base 38..40: mod_base = OTHER; note = Phosphorothioate internucleotide linkage
  misc_feature 1..19: note = DNA
  misc_feature 20..40: note = RNA
  ncRNA 20..40: ncRNA_class = siRNA
cgaagcgccctactccactgtcatcacactgaataccaat

SEQ ID NO: 450 - RNA, 23 nt, synthetic construct
  source 1..23: mol_type = other RNA
  ncRNA 1..23: ncRNA_class = siRNA
  modified_base order(1..3,21..23): mod_base = OTHER; note = Phosphorothioate internucleotide linkage
attggtattcagtgtgatgactt

SEQ ID NO: 451 - DNA, 40 bp, synthetic construct
  source 1..40: mol_type = other DNA
  modified_base 1..3: mod_base = OTHER; note = Phosphorothioate internucleotide linkage
  misc_feature 1..21: note = RNA
  misc_feature 22..40: note = DNA
  ncRNA 1..21: ncRNA_class = siRNA
gtcatcacactgaataccaattcacctcatcccgcgaagc

SEQ ID NO: 452 - DNA, 42 bp, synthetic construct
  misc_feature 1..23: note = RNA
  source 1..42: mol_type = other DNA
  misc_feature 24..42: note = DNA
  ncRNA 1..23: ncRNA_class = siRNA
  modified_base order(1..3,21..23): mod_base = OTHER; note = Phosphorothioate internucleotide linkage
attggtattcagtgtgatgactttcacctcatcccgcgaagc

SEQ ID NO: 453 - RNA, 21 nt, synthetic construct
  source 1..21: mol_type = other RNA
  ncRNA 1..21: ncRNA_class = siRNA
  modified_base order(1..3,19..21): mod_base = OTHER; note = Phosphorothioate internucleotide linkage
gtcatcacactgaataccaat

SEQ ID NO: 454 - DNA, 40 bp, synthetic construct
  source 1..40: mol_type = other DNA
  modified_base 38..40: mod_base = OTHER; note = Phosphorothioate internucleotide linkage
  misc_feature 1..19: note = DNA
  misc_feature 20..40: note = RNA
  ncRNA 20..40: ncRNA_class = siRNA
cgaagcgccctactccactggtgtatggcttcaaccctga

SEQ ID NO: 455 - RNA, 23 nt, synthetic construct
  source 1..23: mol_type = other RNA
  modified_base order(1..3,21..23): mod_base = OTHER; note = Phosphorothioate internucleotide linkage
  ncRNA 1..23: ncRNA_class = siRNA
tcagggttgaagccatacacctt

SEQ ID NO: 456 - DNA, 40 bp, synthetic construct
  source 1..40: mol_type = other DNA
  modified_base 1..3: mod_base = OTHER; note = Phosphorothioate internucleotide linkage
  misc_feature 1..21: note = RNA
  ncRNA 1..21: ncRNA_class = siRNA
  misc_feature 22..40: note = DNA
ggtgtatggcttcaaccctgatcacctcatcccgcgaagc

SEQ ID NO: 457 - RNA, 21 nt, synthetic construct
  source 1..21: mol_type = other RNA
  modified_base order(1..3,19..21): mod_base = OTHER; note = Phosphorothioate internucleotide linkage
  ncRNA 1..21: ncRNA_class = siRNA
ggtgtatggcttcaaccctga

SEQ ID NO: 458 - DNA, 42 bp, synthetic construct
  source 1..42: mol_type = other DNA
  misc_feature 1..23: note = RNA
  misc_feature 24..42: note = DNA
  ncRNA 1..23: ncRNA_class = siRNA
  modified_base order(1..3,21..23): mod_base = OTHER; note = Phosphorothioate internucleotide linkage
tcagggttgaagccatacacctttcacctcatcccgcgaagc

SEQ ID NO: 459 - DNA, 40 bp, synthetic construct
  source 1..40: mol_type = other DNA
  modified_base 38..40: mod_base = OTHER; note = Phosphorothioate internucleotide linkage
  misc_feature 1..19: note = DNA
  misc_feature 20..40: note = RNA
  ncRNA 20..40: ncRNA_class = siRNA
cgaagcgccctactccactcaccaacttcttccacgagtc

SEQ ID NO: 460 - RNA, 23 nt, synthetic construct
  source 1..23: mol_type = other RNA
  ncRNA 1..23: ncRNA_class = siRNA
  modified_base order(1..3,21..23): mod_base = OTHER; note = Phosphorothioate internucleotide linkage
gactcgtggaagaagttggtgtt

SEQ ID NO: 461 - DNA, 40 bp, synthetic construct
  source 1..40: mol_type = other DNA
  modified_base 1..3: mod_base = OTHER; note = Phosphorothioate internucleotide linkage
  misc_feature 1..21: note = RNA
  misc_feature 22..40: note = DNA
  ncRNA 1..21: ncRNA_class = siRNA
caccaacttcttccacgagtctcacctcatcccgcgaagc

SEQ ID NO: 462 - RNA, 21 nt, synthetic construct
  source 1..21: mol_type = other RNA
  modified_base order(1..3,19..21): mod_base = OTHER; note = Phosphorothioate internucleotide linkage
  ncRNA 1..21: ncRNA_class = siRNA
caccaacttcttccacgagtc

SEQ ID NO: 463 - DNA, 42 bp, synthetic construct
  source 1..42: mol_type = other DNA
  misc_feature 1..23: note = RNA
  misc_feature 24..42: note = DNA
  modified_base order(1..3,21..23): mod_base = OTHER; note = Phosphorothioate internucleotide linkage
  ncRNA 1..23: ncRNA_class = siRNA
gactcgtggaagaagttggtgtttcacctcatcccgcgaagc

SEQ ID NO: 464 - DNA, 38 bp, synthetic construct
  source 1..38: mol_type = other DNA
  misc_feature 1..19: note = DNA
  misc_feature 20..38: note = RNA
  modified_base 36..38: mod_base = OTHER; note = Phosphorothioate internucleotide linkage
  ncRNA 20..38: ncRNA_class = siRNA
cgaagcgccctactccactaggcagagctagtggcaaa

SEQ ID NO: 465 - RNA, 21 nt, synthetic construct
  source 1..21: mol_type = other RNA
  modified_base order(1..3,19..21): mod_base = OTHER; note = Phosphorothioate internucleotide linkage
  ncRNA 1..21: ncRNA_class = siRNA
tttgccactagctctgccttt

SEQ ID NO: 466 - DNA, 38 bp, synthetic construct
  source 1..38: mol_type = other DNA
  misc_feature 1..3: note = Phosphorothioate internucleotide linkage
  misc_feature 1..19: note = RNA
  misc_feature 20..38: note = DNA
  ncRNA 1..19: ncRNA_class = siRNA
aggcagagctagtggcaaatcacctcatcccgcgaagc

SEQ ID NO: 467 - RNA, 19 nt, synthetic construct
  source 1..19: mol_type = other RNA
  modified_base order(1..3,17..19): mod_base = OTHER; note = Phosphorothioate internucleotide linkage
  ncRNA 1..19: ncRNA_class = siRNA
aggcagagctagtggcaaa

SEQ ID NO: 468 - DNA, 40 bp, synthetic construct
  source 1..40: mol_type = other DNA
  misc_feature 1..21: note = RNA
  misc_feature 22..40: note = DNA
  ncRNA 1..21: ncRNA_class = siRNA
  modified_base order(1..3,19..21): mod_base = OTHER; note = Phosphorothioate internucleotide linkage
tttgccactagctctgccttttcacctcatcccgcgaagc

SEQ ID NO: 469 - DNA, 38 bp, synthetic construct
  source 1..38: mol_type = other DNA
  misc_feature 1..19: note = DNA
  misc_feature 20..38: note = RNA
  ncRNA 20..38: ncRNA_class = siRNA
  modified_base 36..38: mod_base = OTHER; note = Phosphorothioate internucleotide linkage
cgaagcgccctactccactgagcaaatctcttcaataa

SEQ ID NO: 470 - RNA, 21 nt, synthetic construct
  source 1..21: mol_type = other RNA
  modified_base order(1..3,19..21): mod_base = OTHER; note = Phosphorothioate internucleotide linkage
  ncRNA 1..21: ncRNA_class = siRNA
ttattgaagagatttgctctt

SEQ ID NO: 471 - DNA, 38 bp, synthetic construct
  source 1..38: mol_type = other DNA
  modified_base 1..3: mod_base = OTHER; note = Phosphorothioate internucleotide linkage
  misc_feature 1..19: note = RNA
  misc_feature 20..38: note = DNA
  ncRNA 1..19: ncRNA_class = siRNA
gagcaaatctcttcaataatcacctcatcccgcgaagc

SEQ ID NO: 472 - RNA, 19 nt, synthetic construct
  source 1..19: mol_type = other RNA
  ncRNA 1..19: ncRNA_class = siRNA
  modified_base order(1..3,17..19): mod_base = OTHER; note = Phosphorothioate internucleotide linkage
gagcaaatctcttcaataa

SEQ ID NO: 473 - DNA, 40 bp, synthetic construct
  source 1..40: mol_type = other DNA
  misc_feature 1..21: note = RNA
  misc_feature 22..40: note = DNA
  modified_base order(1..3,19..21): mod_base = OTHER; note = Phosphorothioate internucleotide linkage
  ncRNA 1..21: ncRNA_class = siRNA
ttattgaagagatttgctctttcacctcatcccgcgaagc

SEQ ID NO: 474 - DNA, 38 bp, synthetic construct
  source 1..38: mol_type = other DNA
  misc_feature 1..19: note = DNA
  misc_feature 20..38: note = RNA
  modified_base 36..38: mod_base = OTHER; note = Phosphorothioate internucleotide linkage
  ncRNA 20..38: ncRNA_class = siRNA
cgaagcgccctactccactccacaaatgtctacagcaa

SEQ ID NO: 475 - RNA, 21 nt, synthetic construct
  source 1..21: mol_type = other RNA
  modified_base order(1..3,19..21): mod_base = OTHER; note = Phosphorothioate internucleotide linkage
  ncRNA 1..21: ncRNA_class = siRNA
ttgctgtagacatttgtggtt

SEQ ID NO: 476 - DNA, 38 bp, synthetic construct
  source 1..38: mol_type = other DNA
  modified_base 1..3: mod_base = OTHER; note = Phosphorothioate internucleotide linkage
  misc_feature 1..19: note = RNA
  misc_feature 20..38: note = DNA
  ncRNA 1..19: ncRNA_class = siRNA
ccacaaatgtctacagcaatcacctcatcccgcgaagc

SEQ ID NO: 477 - RNA, 19 nt, synthetic construct
  source 1..19: mol_type = other RNA
  modified_base order(1..3,17..19): mod_base = OTHER; note = Phosphorothioate internucleotide linkage
  ncRNA 1..19: ncRNA_class = siRNA
ccacaaatgtctacagcaa

SEQ ID NO: 478 - DNA, 40 bp, synthetic construct
  source 1..40: mol_type = other DNA
  misc_feature 1..21: note = RNA
  misc_feature 22..40: note = DNA
  modified_base order(1..3,19..21): mod_base = OTHER; note = Phosphorothioate internucleotide linkage
  ncRNA 1..21: ncRNA_class = siRNA
ttgctgtagacatttgtggtttcacctcatcccgcgaagc

SEQ ID NO: 479 - DNA, 38 bp, synthetic construct
  source 1..38: mol_type = other DNA
  misc_feature 1..19: note = DNA
  misc_feature 20..38: note = RNA
  ncRNA 20..38: ncRNA_class = siRNA
cgaagcgccctactccactgaaacaggcttgaaagaat

SEQ ID NO: 480 - RNA, 21 nt, synthetic construct
  source 1..21: mol_type = other RNA
  ncRNA 1..21: ncRNA_class = siRNA
  modified_base order(1..3,19..21): mod_base = OTHER; note = Phosphorothioate internucleotide linkage
attctttcaagcctgtttctt

SEQ ID NO: 481 - DNA, 38 bp, synthetic construct
  source 1..38: mol_type = other DNA
  modified_base 1..3: mod_base = OTHER; note = Phosphorothioate internucleotide linkage
  misc_feature 1..19: note = RNA
  misc_feature 20..38: note = DNA
  ncRNA 1..19: ncRNA_class = siRNA
gaaacaggcttgaaagaattcacctcatcccgcgaagc

SEQ ID NO: 482 - RNA, 19 nt, synthetic construct
  source 1..19: mol_type = other RNA
  ncRNA 1..19: ncRNA_class = siRNA
  modified_base order(1..3,17..19): mod_base = OTHER; note = Phosphorothioate internucleotide linkage
gaaacaggcttgaaagaat

SEQ ID NO: 483 - DNA, 40 bp, synthetic construct
  source 1..40: mol_type = other DNA
  misc_feature 1..21: note = RNA
  misc_feature 22..40: note = DNA
  ncRNA 1..19: ncRNA_class = siRNA
  modified_base order(1..3,19..21): mod_base = OTHER; note = Phosphorothioate internucleotide linkage
attctttcaagcctgtttctttcacctcatcccgcgaagc

SEQ ID NO: 484 - DNA, 38 bp, synthetic construct
  source 1..38: mol_type = other DNA
  misc_feature 1..19: note = DNA
  misc_feature 20..38: note = RNA
  modified_base 36..38: mod_base = OTHER; note = Phosphorothioate internucleotide linkage
  ncRNA 20..38: ncRNA_class = siRNA
cgaagcgccctactccactgagagaaatcgaagaggaa

SEQ ID NO: 485 - RNA, 21 nt, synthetic construct
  source 1..21: mol_type = other RNA
  ncRNA 1..21: ncRNA_class = siRNA
  modified_base order(1..3,19..21): mod_base = OTHER; note = Phosphorothioate internucleotide linkage
ttcctcttcgatttctctctt

SEQ ID NO: 486 - DNA, 38 bp, synthetic construct
  source 1..38: mol_type = other DNA
  modified_base 1..3: mod_base = OTHER; note = Phosphorothioate internucleotide linkage
  misc_feature 1..19: note = RNA
  misc_feature 20..38: note = DNA
  ncRNA 1..19: ncRNA_class = siRNA
gagagaaatcgaagaggaatcacctcatcccgcgaagc

SEQ ID NO: 487 - RNA, 19 nt, synthetic construct
  source 1..19: mol_type = other RNA
  ncRNA 1..19: ncRNA_class = siRNA
  modified_base order(1..3,17..19): mod_base = OTHER; note = Phosphorothioate internucleotide linkage
gagagaaatcgaagaggaa

SEQ ID NO: 488 - DNA, 40 bp, synthetic construct
  source 1..40: mol_type = other DNA
  misc_feature 1..21: note = RNA
  misc_feature 22..40: note = DNA
  ncRNA 1..21: ncRNA_class = siRNA
  modified_base order(1..3,19..21): mod_base = OTHER; note = Phosphorothioate internucleotide linkage
ttcctcttcgatttctctctttcacctcatcccgcgaagc

SEQ ID NO: 489 - DNA, 38 bp, synthetic construct
  source 1..38: mol_type = other DNA
  modified_base 36..38: mod_base = OTHER; note = Phosphorothioate internucleotide linkage
  misc_feature 1..19: note = DNA
  misc_feature 20..38: note = RNA
  ncRNA 20..38: ncRNA_class = siRNA
cgaagcgccctactccactagttatagtccgtgagcta

SEQ ID NO: 490 - RNA, 21 nt, synthetic construct
  source 1..21: mol_type = other RNA
  modified_base order(1..3,19..21): mod_base = OTHER; note = Phosphorothioate internucleotide linkage
  ncRNA 1..21: ncRNA_class = siRNA
tagctcacggactataacttt

SEQ ID NO: 491 - DNA, 38 bp, synthetic construct
  source 1..38: mol_type = other DNA
  misc_feature 1..19: note = RNA
  misc_feature 20..38: note = DNA
  modified_base 1..3: mod_base = OTHER; note = Phosphorothioate internucleotide linkage
  ncRNA 1..19: ncRNA_class = siRNA
agttatagtccgtgagctatcacctcatcccgcgaagc

SEQ ID NO: 492 - RNA, 19 nt, synthetic construct
  source 1..19: mol_type = other RNA
  modified_base order(1..3,17..19): mod_base = OTHER; note = Phosphorothioate internucleotide linkage
  ncRNA 1..19: ncRNA_class = siRNA
agttatagtccgtgagcta

SEQ ID NO: 493 - DNA, 40 bp, synthetic construct
  source 1..40: mol_type = other DNA
  misc_feature 1..21: note = RNA
  misc_feature 22..40: note = DNA
  ncRNA 1..21: ncRNA_class = siRNA
  modified_base order(1..3,19..21): mod_base = OTHER; note = Phosphorothioate internucleotide linkage
tagctcacggactataacttttcacctcatcccgcgaagc

SEQ ID NO: 494 - RNA, 21 nt, synthetic construct
  source 1..21: mol_type = other RNA
  modified_base 1..3: mod_base = OTHER; note = Phosphorothioate internucleotide linkage
  modified_base 11: mod_base = OTHER; note = thymine
  modified_base order(1..6,8,10,12..21): mod_base = OTHER; note = 2-Prime-O-methyl ribonucleotides
  modified_base order(7,9): mod_base = OTHER; note = 2-Prime-deoxy-2-Prime-fluoro ribonucleotides
  ncRNA 1..21: ncRNA_class = siRNA
ctagacctgttttgcttttgt

SEQ ID NO: 495 - RNA, 23 nt, synthetic construct
  source 1..23: mol_type = other RNA
  modified_base order(1..3,21..23): mod_base = OTHER; note = Phosphorothioate internucleotide linkage
  modified_base order(1,3,7,9,11,13,15,17,19..23): mod_base = OTHER; note = 2-Prime-O-methyl ribonucleotides
  modified_base order(2,4..6,8,10,12,14,16,18): mod_base = OTHER; note = 2-Prime-deoxy-2-Prime-fluoro ribonucleotides
  ncRNA 1..23: ncRNA_class = siRNA
acaaaagcaaaacaggtctagaa

SEQ ID NO: 496 - DNA, 40 bp, synthetic construct
  source 1..40: mol_type = other DNA
  misc_feature 1..21: note = RNA
  misc_feature 22..40: note = DNA
  modified_base 11: mod_base = OTHER; note = thymine
  ncRNA 1..21: ncRNA_class = siRNA
ctagacctgttttgcttttgttcacctcatcccgcgaagc

SEQ ID NO: 497 - DNA, 40 bp, synthetic construct
  source 1..40: mol_type = other DNA
  misc_feature 1..19: note = DNA
  misc_feature 20..40: note = RNA
  ncRNA 20..40: ncRNA_class = siRNA
  modified_base 30: mod_base = OTHER; note = thymine
cgaagcgccctactccactctagacctgttttgcttttgt

SEQ ID NO: 498 - DNA, 40 bp, synthetic construct
  source 1..40: mol_type = other DNA
  modified_base 30: mod_base = OTHER; note = thymine
  misc_feature 1..19: note = DNA
  misc_feature 20..40: note = RNA
  ncRNA 20..40: ncRNA_class = siRNA
gcgaagcccctactccactctagacctgttttgcttttgt

SEQ ID NO: 499 - RNA, 21 nt, synthetic construct
  misc_feature 1..21: note = artificial sequence
  source 1..21: mol_type = other RNA
  modified_base 19..21: mod_base = OTHER; note = Phosphorothioate internucleotide linkage
gtcatcacactgaataccaat

SEQ ID NO: 500 - RNA, 21 nt, synthetic construct
  misc_feature 1..21: note = artificial sequence
  source 1..21: mol_type = other RNA
  modified_base 1..3: mod_base = OTHER; note = Phosphorothioate internucleotide linkage
gtcatcacactgaataccaat

SEQ ID NO: 501 - RNA, 23 nt, synthetic construct
  misc_feature 1..23: note = artificial sequence
  source 1..23: mol_type = other RNA
  modified_base order(1..3,21..23): mod_base = OTHER; note = Phosphorothioate internucleotide linkage
attggtattcagtgtgatgactt

SEQ ID NO: 502 - RNA, 21 nt, synthetic construct
  misc_feature 1..21: note = artificial sequence
  source 1..21: mol_type = other RNA
  modified_base 19..21: mod_base = OTHER; note = Phosphorothioate internucleotide linkage
ggtgtatggcttcaaccctga

SEQ ID NO: 503 - RNA, 21 nt, synthetic construct
  misc_feature 1..21: note = artificial sequence
  source 1..21: mol_type = other RNA
  modified_base 1..3: mod_base = OTHER; note = Phosphorothioate internucleotide linkage
ggtgtatggcttcaaccctga

SEQ ID NO: 504 - RNA, 23 nt, synthetic construct
  misc_feature 1..23: note = artificial sequence
  source 1..23: mol_type = other RNA
  modified_base order(1..3,21..23): mod_base = OTHER; note = Phosphorothioate internucleotide linkage
tcagggttgaagccatacacctt

SEQ ID NO: 505 - RNA, 21 nt, synthetic construct
  misc_feature 1..21: note = artificial sequence
  source 1..21: mol_type = other RNA
  modified_base 19..21: mod_base = OTHER; note = Phosphorothioate internucleotide linkage
caccaacttcttccacgagtc

SEQ ID NO: 506 - RNA, 21 nt, synthetic construct
  misc_feature 1..21: note = artificial sequence
  source 1..21: mol_type = other RNA
  modified_base 1..3: mod_base = OTHER; note = Phosphorothioate internucleotide linkage
caccaacttcttccacgagtc

SEQ ID NO: 507 - RNA, 23 nt, synthetic construct
  misc_feature 1..23: note = artificial sequence
  source 1..23: mol_type = other RNA
  modified_base order(1..3,21..23): mod_base = OTHER; note = Phosphorothioate internucleotide linkage
gactcgtggaagaagttggtgtt

SEQ ID NO: 508 - RNA, 19 nt, synthetic construct
  misc_feature 1..19: note = artificial sequence
  source 1..19: mol_type = other RNA
  modified_base 17..19: mod_base = OTHER; note = Phosphorothioate internucleotide linkage
aggcagagctagtggcaaa

SEQ ID NO: 509 - RNA, 19 nt, synthetic construct
  misc_feature 1..19: note = artificial sequence
  source 1..19: mol_type = other RNA
  modified_base 1..3: mod_base = OTHER; note = Phosphorothioate internucleotide linkage
aggcagagctagtggcaaa

SEQ ID NO: 510 - RNA, 21 nt, synthetic construct
  misc_feature 1..21: note = artificial sequence
  source 1..21: mol_type = other RNA
  modified_base order(1..3,19..21): mod_base = OTHER; note = Phosphorothioate internucleotide linkage
tttgccactagctctgccttt

SEQ ID NO: 511 - RNA, 19 nt, synthetic construct
  misc_feature 1..19: note = artificial sequence
  source 1..19: mol_type = other RNA
  modified_base 17..19: mod_base = OTHER; note = Phosphorothioate internucleotide linkage
gagcaaatctcttcaataa

SEQ ID NO: 512 - RNA, 19 nt, synthetic construct
  misc_feature 1..19: note = artificial sequence
  source 1..19: mol_type = other RNA
  modified_base 1..3: mod_base = OTHER; note = Phosphorothioate internucleotide linkage
gagcaaatctcttcaataa

SEQ ID NO: 513 - RNA, 21 nt, synthetic construct
  misc_feature 1..21: note = artificial sequence
  source 1..21: mol_type = other RNA
  modified_base order(1..3,19..21): mod_base = OTHER; note = Phosphorothioate internucleotide linkage
ttattgaagagatttgctctt

SEQ ID NO: 514 - RNA, 19 nt, synthetic construct
  misc_feature 1..19: note = artificial sequence
  source 1..19: mol_type = other RNA
  modified_base 17..19: mod_base = OTHER; note = Phosphorothioate internucleotide linkage
ccacaaatgtctacagcaa

SEQ ID NO: 515 - RNA, 19 nt, synthetic construct
  misc_feature 1..19: note = artificial sequence
  source 1..19: mol_type = other RNA
  modified_base 1..3: mod_base = OTHER; note = Phosphorothioate internucleotide linkage
ccacaaatgtctacagcaa

SEQ ID NO: 516 - RNA, 21 nt, synthetic construct
  misc_feature 1..21: note = artificial sequence
  source 1..21: mol_type = other RNA
  modified_base order(1..3,19..21): mod_base = OTHER; note = Phosphorothioate internucleotide linkage
ttgctgtagacatttgtggtt

SEQ ID NO: 517 - RNA, 19 nt, synthetic construct
  misc_feature 1..19: note = artificial sequence
  source 1..19: mol_type = other RNA
  modified_base 17..19: mod_base = OTHER; note = Phosphorothioate internucleotide linkage
gaaacaggcttgaaagaat

SEQ ID NO: 518 - RNA, 19 nt, synthetic construct
  misc_feature 1..19: note = artificial sequence
  source 1..19: mol_type = other RNA
  modified_base 1..3: mod_base = OTHER; note = Phosphorothioate internucleotide linkage
gaaacaggcttgaaagaat

SEQ ID NO: 519 - RNA, 21 nt, synthetic construct
  misc_feature 1..21: note = artificial sequence
  source 1..21: mol_type = other RNA
  modified_base order(1..3,19..21): mod_base = OTHER; note = Phosphorothioate internucleotide linkage
attctttcaagcctgtttctt

SEQ ID NO: 520 - RNA, 19 nt, synthetic construct
  misc_feature 1..19: note = artificial sequence
  source 1..19: mol_type = other RNA
  modified_base 17..19: mod_base = OTHER; note = Phosphorothioate internucleotide linkage
gagagaaatcgaagaggaa

SEQ ID NO: 521 - RNA, 19 nt, synthetic construct
  misc_feature 1..19: note = artificial sequence
  source 1..19: mol_type = other RNA
  modified_base 1..3: mod_base = OTHER; note = Phosphorothioate internucleotide linkage
gagagaaatcgaagaggaa

SEQ ID NO: 522 - RNA, 21 nt, synthetic construct
  misc_feature 1..21: note = artificial sequence
  source 1..21: mol_type = other RNA
  modified_base order(1..3,19..21): mod_base = OTHER; note = Phosphorothioate internucleotide linkage
ttcctcttcgatttctctctt

SEQ ID NO: 523 - RNA, 19 nt, synthetic construct
  misc_feature 1..19: note = artificial sequence
  source 1..19: mol_type = other RNA
  modified_base 17..19: mod_base = OTHER; note = Phosphorothioate internucleotide linkage
agttatagtccgtgagcta

SEQ ID NO: 524 - RNA, 19 nt, synthetic construct
  misc_feature 1..19: note = artificial sequence
  source 1..19: mol_type = other RNA
  modified_base 1..3: mod_base = OTHER; note = Phosphorothioate internucleotide linkage
agttatagtccgtgagcta

SEQ ID NO: 525 - RNA, 21 nt, synthetic construct
  misc_feature 1..21: note = artificial sequence
  source 1..21: mol_type = other RNA
  modified_base order(1..3,19..21): mod_base = OTHER; note = Phosphorothioate internucleotide linkage
tagctcacggactataacttt

SEQ ID NO: 526 - DNA, 28 bp, synthetic construct
  misc_feature 8..28: note = RNA
  source 1..28: mol_type = other DNA
  misc_feature 1..7: note = DNA
cgaagcgcaggtctggaatgcaaagtca

SEQ ID NO: 527 - RNA, 23 nt, synthetic construct
  source 1..23: mol_type = other RNA
  ncRNA 1..23: ncRNA_class = siRNA
  modified_base order(1..3,21..23): mod_base = OTHER; note = Phosphorothioate internucleotide linkage
ttccgaataaactccaggccttt

SEQ ID NO: 528 - RNA, 23 nt, synthetic construct
  source 1..23: mol_type = other RNA
  ncRNA 1..23: ncRNA_class = siRNA
  modified_base order(1..3,21..23): mod_base = OTHER; note = Phosphorothioate internucleotide linkage
ttccgaataaactccaggccttt